(19) United States
(12) Patent Application Publication
(10) Pub. No.: US 2024/0279309 A1
(43) Pub. Date: Aug. 22, 2024

(54) RECOMBINANT CD40 BINDING PROTEINS AND THEIR USE

(71) Applicant: MOLECULAR PARTNERS AG, Zürich-Schlieren (CH)

(72) Inventors: Valérie Perrine CALABRO, Bergdietikon (CH); Nicolo RIGAMONTI, Zürich (CH); Clara DOMKE, Dietikon (CH); Anja Christina SCHLEGEL, Schlieren (CH)

(21) Appl. No.: 17/925,138

(22) PCT Filed: May 14, 2021
(86) PCT No.: PCT/EP2021/062874

§ 371 Date: Nov. 14, 2022

(30) Foreign Application Priority Data
May 14, 2020 (EP) 20174830.8

(51) Int. Cl. C07K 14/705; A61K 38/00

(52) U.S. Cl. CPC C07K 14/70575; A61K 38/00; C07K 2319/01

(57) ABSTRACT

The present invention relates to recombinant binding proteins comprising a designed ankyrin repeat domain with binding specificity for CD40. In addition, the invention relates to nucleic acids encoding such binding proteins, pharmaceutical compositions comprising such binding proteins or nucleic acids, and the use of such binding proteins, nucleic acids or pharmaceutical compositions in methods for activating CD40 in cells expressing CD40, e.g., tumor-localized B-cells, and for treating or diagnosing diseases, such as cancer, in a mammal, including a human.

### CROSS-REFERENCE TO RELATED APPLICATION

The present application claims the benefit of and priority from European patent application EP20174830 filed on 14 May 2020 with the European Patent Office. The content of European patent application EP20174830 is incorporated herein by reference in its entirety, including all tables, figures, and claims.

### FIELD OF THE DISCLOSURE

The present invention relates to recombinant binding proteins comprising a designed ankyrin repeat domain with binding specificity for CD40. In addition, the invention relates to nucleic acids encoding such binding proteins, pharmaceutical compositions comprising such binding proteins or nucleic acids, and the use of such binding proteins, nucleic acids or pharmaceutical compositions in methods for activating CD40 in cells expressing CD40, e.g., tumor-localized B-cells, and for treating or diagnosing diseases, such as cancer, in a mammal, including a human.

### BACKGROUND

Tumor necrosis factor receptor (TNFR) superfamily member CD40 is a key co-stimulatory receptor, and when engaged by its ligand (CD40L) or by agonistic antibodies, it is involved in the regulation of a wide spectrum of molecular and cellular processes, including the initiation and progression of cellular and humoral adaptive immunity. For example, it has been demonstrated that CD40 engagement on the surface of dendritic cells promotes their cytokine production, induces the expression of costimulatory molecules on their surface, and facilitates the presentation of antigen. Overall, the impact of CD40 signaling ‘licenses’ dendritic cells to mature and achieve all of the necessary characteristics to effectively trigger T-cell activation and differentiation. CD40 signaling in B cells promotes germinal center formation, immunoglobulin (Ig) isotype switching, somatic hypermutation of the Ig to enhance affinity for antigen, and finally the formation of long-lived plasma cells and memory B cells. Moreover, it has been shown that the CD40 pathway is important for the survival of many cell types including germinal center B cells, dendritic cells, and endothelial cells under normal and inflammatory conditions. Deregulation of CD40 signaling has been observed in various autoimmune diseases. Together, this breadth of functions underlines the importance of the CD40 receptor for the generation of an acquired immune response.

CD40 was initially characterized on B cells and is also expressed on dendritic cells, monocytes, platelets, and macrophages as well as on non-hematopoietic cells such as myofibroblasts, fibroblasts, epithelial, and endothelial cells. The ligand of CD40, known as CD154 or CD40L, is expressed primarily by activated T cells, as well as activated B cells and platelets, and under inflammatory conditions it is also induced in monocytic cells, natural killer cells, mast cells, and basophils.

Because CD40 can activate both the innate and adaptive immune system, it has been recognized as a suitable target for tumor immunotherapy. Several reports have confirmed that CD40 stimulation can enhance anti-tumor immune responses by means of dendritic cell maturation. Activation of dendritic cells with agonists of CD40 results in their increased survival, secretion of IL-1, IL-6, IL-8, IL-12, TNF-α, and macrophage inflammatory protein-1α. Additionally, CD40 activation induces the upregulation of costimulatory molecules such as MHC class II, LFA-3, CD80, and CD86 and promotes antigen presentation, priming and cross-priming of T helper cells (Th) and cytotoxic T lymphocytes (CTL), respectively. Agonistic antibodies against CD40 have proved efficacious in preclinical murine tumor models. However, although their use in the clinic has shown some anti-tumor efficacy, clinical development of agonistic anti-CD40 antibodies has likely been hampered by dose-limiting toxicities and resulting low efficacies.

Thus, there remains a need for new CD40-specific binding proteins, and for therapeutic and diagnostic approaches for the treatment and characterization of diseases, including cancer, benefitting from CD40-specific binding and activation. In particular, there is a need for new CD40-specific binding proteins that can function as effective agonists of CD40 and that can also easily be combined with other functional moieties, such as, e.g., a localizer molecule.

### SUMMARY

The present invention provides recombinant binding proteins comprising a designed ankyrin repeat domain with binding specificity for CD40. Further provided are such binding proteins linked to one or more localizer molecules, which facilitate clustering-mediated activation of CD40 by the binding proteins. In addition, the invention provides nucleic acids encoding such binding proteins and pharmaceutical compositions comprising such binding proteins or nucleic acids. The invention also provides the use of such binding proteins, nucleic acids or pharmaceutical compositions in methods for localized activation of CD40 in CD40-expressing cells or tissue, such as tumor tissue, and for treating and diagnosing diseases, such as cancer, in a mammal, including a human.

In one aspect, the invention provides such a recombinant binding protein comprising an ankyrin repeat domain with binding specificity for CD40, wherein said ankyrin repeat domain comprises an ankyrin repeat module comprising an amino acid sequence selected from the group consisting of (1) SEQ ID NOs: 39 to 95 and (2) sequences in which up to 10 amino acids in any of SEQ ID NOs: 39 to 95 are substituted by other amino acids. As an example, in one particular embodiment, the CD40-specific recombinant binding protein of the invention comprises an ankyrin repeat domain with binding specificity for CD40, wherein said ankyrin repeat domain comprises an ankyrin repeat module comprising an amino acid sequence selected from the group consisting of (1) SEQ ID NOs: 76, 77, and 78 and (2) sequences in which up to 10 amino acids in any of SEQ ID NOs: 76, 77, and 78 are substituted by other amino acids. In one particular embodiment, the CD40-specific recombinant binding protein of the invention comprises an ankyrin repeat domain with binding specificity for CD40, wherein said ankyrin repeat domain comprises a first ankyrin repeat module comprising an amino acid sequence selected from the group consisting of (1) SEQ ID NO: 76 and (2) sequences in which up to 10 amino acids in SEQ ID NO: 76 are substituted by other amino acids, and wherein said ankyrin repeat domain further comprises (i) a second ankyrin repeat module comprising an amino acid sequence selected from the group consisting of (1) SEQ ID NO: 77 and (2) sequences in which up to 10 amino acids in SEQ ID NO: 77 are substituted by other amino acids and (ii) a third ankyrin repeat module comprising an amino acid sequence selected from the group consisting of (1) SEQ ID NO: 78 and (2) sequences in which up to 10 amino acids in SEQ ID NO: 78 are substituted by other amino acids. Preferably, said first ankyrin repeat module is located N-terminally of said second ankyrin repeat module, and said second ankyrin repeat module is located N-terminally of said third ankyrin repeat module within said ankyrin repeat domain. Even more preferably, said ankyrin repeat domain further comprises (iii) an N-terminal capping module, wherein said N-terminal capping module comprises an amino acid sequence selected from the group consisting of (1) SEQ ID NOs: 5 to 8 and (2) sequences in which up to 10 amino acids in any of SEQ ID NOs: 5 to 7 are substituted by other amino acids, and (iv) a C-terminal capping module, wherein said C-terminal capping module comprises an amino acid sequence selected from the group consisting of (1) SEQ ID NOs: 12 to 15 and (2) sequences in which up to 10 amino acids in any of SEQ ID NOs: 12 to 14 are substituted by other amino acids.

In one aspect, the invention provides such a recombinant binding protein comprising an ankyrin repeat domain with binding specificity for CD40, wherein said ankyrin repeat domain comprises an amino acid sequence with at least 75% and up to 100% amino acid sequence identity with any one of SEQ ID NOs: 16 to 35, wherein G at position 1 and/or S at position 2 of SEQ ID NOs: 16 to 35 are optionally missing. As an example, in one particular embodiment, the CD40-specific recombinant binding protein of the invention comprises an ankyrin repeat domain with binding specificity for CD40, wherein said ankyrin repeat domain comprises the amino acid sequence of SEQ ID NO: 29, wherein G at position 1 and/or S at position 2 of said ankyrin repeat domain are optionally missing. In another particular embodiment, the ankyrin repeat domain with binding specificity for CD40 of the invention specifically binds to the N-terminal cysteine-rich domain (CRD) 1 of the CD40 receptor. The amino acid sequence of the CD40 receptor is herein provided in SEQ ID NO: 96, where the CRD1 corresponds to amino acids 23-59 of SEQ ID NO: 96.

In another aspect, the invention provides such CD40-specific recombinant binding proteins, wherein the binding proteins further comprise a localizer molecule. The localizer molecule may be selected from molecules of different structural and functional classes. For example, a localizer may be a polypeptide binding domain, a cell surface receptor ligand or a fragment or variant thereof, an antibody or a fragment or variant thereof, or an antibody-like protein based on a scaffold. In one aspect of the invention, the localizer molecule is covalently bound to the CD40-specific recombinant binding protein. The covalent bond may be a peptide bond between the CD40-specific binding protein and a localizer peptide or polypeptide, resulting in a fusion protein. Alternatively, the localizer molecule may be covalently conjugated to the CD40-specific binding protein.

In one particular embodiment, a CD40-specific recombinant binding protein of the invention comprises an ankyrin repeat domain with binding specificity for CD40 fused to a localizer with binding specificity for a localizer target protein relevant in cancer biology, such as, e.g., a tumor-associated antigen. As examples, in one particular embodiment, a CD40-specific recombinant binding protein of the invention comprises an ankyrin repeat domain with binding specificity for CD40 fused to another ankyrin repeat domain with binding specificity for fibroblast activation protein (FAP). An example of such an ankyrin repeat domain with binding specificity for FAP is provided in SEQ ID NO: 98.

In another aspect, the invention provides nucleic acids encoding the CD40-specific binding proteins of the invention. In a further aspect, the invention provides pharmaceutical compositions comprising the CD40-specific binding protein or nucleic acid of the invention and a pharmaceutically acceptable carrier and/or diluent.

In another aspect, the invention provides a method of localized activation of CD40 in CD40-expressing cells or tissue in a mammal, the method comprising administering to said mammal the CD40-specific binding protein of the invention comprising a localizer molecule. In one particular embodiment, such method comprises administering the CD40-specific binding protein to a mammal, including a human patient, with a tumor comprising CD40-expressing cells or tissue, resulting in tumor-localized activation of CD40 in the CD40-expressing cells or tumor tissue.

In another aspect, the invention provides a method for treating a medical condition in a human patient, the method comprising administering to said patient the CD40-specific binding protein of the invention covalently linked to or comprising a localizer molecule, wherein the localizer molecule mediates local activation of CD40 by the CD40-specific binding protein. In one particular embodiment, the medical condition is cancer, wherein the cancer or tumor tissue comprises cells that express CD40, and the localizer molecule binds a target selectively expressed or overexpressed in said cancer or tumor tissue. In one particular embodiment, said target is the extracellular domain of a cell surface protein selectively expressed or overexpressed in said cancer or tumor tissue. In one embodiment, said cancer is selected from colorectal cancers, gastric cancers, non-small cell lung cancers, breast cancers, head and neck cancers, ovarian cancers, cervix cancers, lung cancers, invasive bladder cancers, pancreatic cancers, metastatic cancers of the brain, head and neck squamous cell carcinoma, esophagus squamous cell carcinoma, lung squamous cell carcinoma, skin squamous cell carcinoma, urothelial carcinoma, melanoma, breast adenocarcinoma, lung adenocarcinoma, cervix squamous cell carcinoma, pancreas squamous cell carcinoma, colon squamous cell carcinoma, or stomach squamous cell carcinoma, prostate cancer, osteosarcoma or soft tissue sarcoma and benign tumors. In one embodiment, such cancer is selected from epithelial malignancies (primary and metastatic), including lung, colorectal, gastric, bladder, ovarian and breast carcinomas, and bone and soft tissue sarcomas.

The invention further provides a kit comprising the recombinant binding protein of the invention, the nucleic acid of the invention or the pharmaceutical composition of the invention. The invention further provides a method for producing the recombinant binding protein of the invention, the method comprising the steps of (i) expressing said recombinant binding protein in bacteria, and (ii) purifying said recombinant binding protein using chromatography.

### BRIEF DESCRIPTION OF THE FIGURES

FIG. 1. A cartoon depicting the in vitro B cell activation assay. The assay was performed using purified primary human B cells and FAP expressing (+FAP) or non-FAP expressing (−FAP) CHO cells.

FIG. 2. Overview of the gating strategy used to determine MFI and percentage of cells positive for CD86. The following settings were used: FSC: 200; SSC: 400; Acquisition: 200 μl/min, 100.000 events. Abbreviations: FMO=Fluorescence minus one, SSC=Side scatter, FSC=Forward scatter, FSC−A=Forward scatter area, FSC−H=Forward scatter height.

FIG. 3. A CD40-specific binding protein combined with a localizer activates CD40 signaling in a strictly localizer-dependent manner and HSA-binding domain(s) impair potency and efficacy of this multi-functional binding protein. Human B cells were co-cultured in presence of FAP-expressing CHO cells (full symbols) and treated with increasing concentrations of SMA014 (triangle pointing up), SMA087 (triangle pointing down), SMA095 (diamond) and agonist anti-CD40 mAb (square). As control, B cells were co-cultured in presence of FAP-negative CHO cells and treated only with the highest concentration of the respective constructs, depicted as empty symbols. Activation of human B cells was assessed in terms of upregulation of CD86 (measured as mean fluorescence intensity (MFI) and percentage of cells (%)) in absence A) and in presence B) of 600 μM HSA. Each value depicts the average of duplicated measurements. The shown data are representative of two independent experiments. Error bars show ±SEM. EC50 and efficacy values (in nM) for all constructs in presence of FAP-expressing CHO cells are shown in the depicted tables in the graphs.

FIG. 4. CD40 bivalency strongly increases potency and efficacy of a multi-functional CD40-localizer binding protein. Human B cells were cultured in presence of FAP expressing CHO cells and treated with increasing concentrations of SMA014 (triangle pointing up), SMA104 (triangle pointing down), SMA105 (diamond) and agonist anti-CD40 mAb (square). As control, B cells were co-cultured in presence of FAP-negative CHO cells and treated only with the highest concentration of the respective constructs, depicted as empty symbols. Activation of human B cells was assessed in terms of upregulation of CD86 (measured as mean fluorescence intensity (MFI) and percentage of cells (%)) in absence of HSA. Each value depicts the average of duplicated measurements. The shown data are representative of two independent experiments. Error bars show ±SEM. EC50 and efficacy values (in nM) for all constructs in presence of FAP-expressing CHO cells are shown in the depicted tables in the graphs.

FIG. 5. CD40 bivalency rescues the inhibitory effect induced by a HSA binding domain. Human B cells were cultured in presence of FAP expressing CHO cells and treated with increasing concentrations of SMA014 (triangle pointing up), SMA104 (triangle pointing down), SMA091 (circle), SMA099 (diamond), AS579 (hexagon) and agonist anti-CD40 mAb (square). As control, B cells were co-cultured in presence of FAP-negative CHO cells and treated only with the highest concentration of the respective constructs, depicted as empty symbol. Activation of human B cells was assessed in terms of upregulation of CD86 (measured as mean fluorescence intensity (MFI) and percentage of cells (%)) in absence A) and in presence B) of HSA. Each value depicts the average of duplicated measurements. The shown data are representative of two independent experiments. Error bars show ±SEM. EC50 and efficacy values (in nM) for all constructs in presence of FAP expressing CHO cells are shown in the depicted tables in the graphs.

FIG. 6. Structure determination by X-ray crystallography of human tumor necrosis factor receptor superfamily member 5 (hCD40) in complex with DARPin® protein #29 (SEQ ID NO: 29).

### DETAILED DESCRIPTION OF THE INVENTION

As disclosed and exemplified herein, the disclosure provides ankyrin repeat proteins that specifically target CD40. Designed ankyrin repeat protein libraries (WO2002/020565; Binz et al., Nat. Biotechnol. 22, 575-582, 2004; Stumpp et al., Drug Discov. Today 13, 695-701, 2008) can be used for the selection of target-specific designed ankyrin repeat domains that bind to their target with high affinity. Such target-specific designed ankyrin repeat domains in turn can be used as valuable components of recombinant binding proteins for the treatment of diseases. Designed ankyrin repeat proteins are a class of binding molecules which have the potential to overcome limitations of monoclonal antibodies, hence allowing novel therapeutic approaches. Such ankyrin repeat proteins may comprise a single designed ankyrin repeat domain, or may comprise a combination of two or more designed ankyrin repeat domains with the same or different target specificities (Stumpp et al., Drug Discov. Today 13, 695-701, 2008; U.S. Pat. No. 9,458,211). Ankyrin repeat proteins comprising only a single designed ankyrin repeat domain are small proteins (14 kDa) which can be selected to bind a given target protein with high affinity and specificity. These characteristics, and the possibility of combining two or more designed ankyrin repeat domains in one protein, make designed ankyrin repeat proteins ideal agonistic, antagonistic and/or inhibitory drug candidates. Furthermore, such ankyrin repeat proteins can be engineered to carry various effector functions, e.g. cytotoxic agents or half-life extending agents, enabling completely new drug formats. Taken together, designed ankyrin repeat proteins are an example of the next generation of protein therapeutics with the potential to surpass existing antibody drugs.

DARPin® is a trademark owned by Molecular Partners AG, Switzerland.

In one aspect, the invention provides recombinant binding proteins comprising a designed ankyrin repeat domain with binding specificity for CD40. In one embodiment, the recombinant binding protein of the invention comprises an ankyrin repeat domain, wherein said ankyrin repeat domain has binding specificity for CD40, and wherein said ankyrin repeat domain comprises an ankyrin repeat module comprising an amino acid sequence selected from the group consisting of (1) SEQ ID NOs: 39 to 95 and (2) sequences in which up to 10, or up to 9, or up to 8, or up to 7, or up to 6, or up to 5, or up to 4, or up to 3, or up to 2, or up to 1 amino acids in any of SEQ ID NOs: 39 to 95 are substituted by another amino acid. Thus, in one embodiment, said ankyrin repeat module comprises an amino acid sequence selected from the group consisting of (1) SEQ ID NOs: 39 to 95 and (2) sequences in which up to 10 amino acids in any of SEQ ID NOs: 39 to 95 are substituted by another amino acid. In one embodiment, said ankyrin repeat module comprises an amino acid sequence selected from the group consisting of (1) SEQ ID NOs: 39 to 95 and (2) sequences in which up to 9 amino acids in any of SEQ ID NOs: 39 to 95 are substituted by another amino acid. In one embodiment, said ankyrin repeat module comprises an amino acid sequence selected from the group consisting of (1) SEQ ID NOs: 39 to 95 and (2) sequences in which up to 8 amino acids in any of SEQ ID NOs: 39 to 95 are substituted by another amino acid. In one embodiment, said ankyrin repeat module comprises an amino acid sequence selected from the group consisting of (1) SEQ ID NOS: 39 to 95 and (2) sequences in which up to 7 amino acids in any of SEQ ID NOs: 39 to 95 are substituted by another amino acid. In one embodiment, said ankyrin repeat module comprises an amino acid sequence selected from the group consisting of (1) SEQ ID NOs: 39 to 95 and (2) sequences in which up to 6 amino acids in any of SEQ ID NOs: 39 to 95 are substituted by another amino acid. In one embodiment, said ankyrin repeat module comprises an amino acid sequence selected from the group consisting of (1) SEQ ID NOs: 39 to 95 and (2) sequences in which up to 5 amino acids in any of SEQ ID NOs: 39 to 95 are substituted by another amino acid. In one embodiment, said ankyrin repeat module comprises an amino acid sequence selected from the group consisting of (1) SEQ ID NOs: 39 to 95 and (2) sequences in which up to 4 amino acids in any of SEQ ID NOs: 39 to 95 are substituted by another amino acid. In one embodiment, said ankyrin repeat module comprises an amino acid sequence selected from the group consisting of (1) SEQ ID NOs: 39 to 95 and (2) sequences in which up to 3 amino acids in any of SEQ ID NOs: 39 to 95 are substituted by another amino acid. In one embodiment, said ankyrin repeat module comprises an amino acid sequence selected from the group consisting of (1) SEQ ID NOs: 39 to 95 and (2) sequences in which up to 2 amino acids in any of SEQ ID NOs: 39 to 95 are substituted by another amino acid. In one embodiment, said ankyrin repeat module comprises an amino acid sequence selected from the group consisting of (1) SEQ ID NOs: 39 to 95 and (2) sequences in which up to 1 amino acid in any of SEQ ID NOs: 39 to 95 is substituted by another amino acid. In one embodiment, all of said 10, 9, 8, 7, 6, 5, 4, 3, 2, or 1 amino acid substitutions occur in framework positions of said ankyrin repeat module(s). In one preferred embodiment, said ankyrin repeat module comprises an amino acid sequence selected from the group consisting of SEQ ID NOs: 39 to 95.

In one embodiment, the recombinant binding protein of the invention comprises an ankyrin repeat domain, wherein said ankyrin repeat domain has binding specificity for CD40, and wherein said ankyrin repeat domain comprises an ankyrin repeat module comprising an amino acid sequence selected from the group consisting of (1) SEQ ID NOs: 56 to 58, 76 to 78 and 81 to 83 and (2) sequences in which up to 10, or up to 9, or up to 8, or up to 7, or up to 6, or up to 5, or up to 4, or up to 3, or up to 2, or up to 1 amino acids in any of SEQ ID NOs: 56 to 58, 76 to 78 and 81 to 83 are substituted by another amino acid. Thus, in one embodiment, said ankyrin repeat module comprises an amino acid sequence selected from the group consisting of (1) SEQ ID NOs: 56 to 58, 76 to 78 and 81 to 83 and (2) sequences in which up to 10 amino acids in any of SEQ ID NOs: 56 to 58, 76 to 78 and 81 to 83 are substituted by another amino acid. In one embodiment, said ankyrin repeat module comprises an amino acid sequence selected from the group consisting of (1) SEQ ID NOs: 56 to 58, 76 to 78 and 81 to 83 and (2) sequences in which up to 9 amino acids in any of SEQ ID NOs: 56 to 58, 76 to 78 and 81 to 83 are substituted by another amino acid. In one embodiment, said ankyrin repeat module comprises an amino acid sequence selected from the group consisting of (1) SEQ ID NOs: 56 to 58, 76 to 78 and 81 to 83 and (2) sequences in which up to 8 amino acids in any of SEQ ID NOs: 56 to 58, 76 to 78 and 81 to 83 are substituted by another amino acid. In one embodiment, said ankyrin repeat module comprises an amino acid sequence selected from the group consisting of (1) SEQ ID NOs: 56 to 58, 76 to 78 and 81 to 83 and (2) sequences in which up to 7 amino acids in any of SEQ ID NOs: 56 to 58, 76 to 78 and 81 to 83 are substituted by another amino acid. In one embodiment, said ankyrin repeat module comprises an amino acid sequence selected from the group consisting of (1) SEQ ID NOs: 56 to 58, 76 to 78 and 81 to 83 and (2) sequences in which up to 6 amino acids in any of SEQ ID NOs: 56 to 58, 76 to 78 and 81 to 83 are substituted by another amino acid. In one embodiment, said ankyrin repeat module comprises an amino acid sequence selected from the group consisting of (1) SEQ ID NOs: 56 to 58, 76 to 78 and 81 to 83 and (2) sequences in which up to 5 amino acids in any of SEQ ID NOs: 56 to 58, 76 to 78 and 81 to 83 are substituted by another amino acid. In one embodiment, said ankyrin repeat module comprises an amino acid sequence selected from the group consisting of (1) SEQ ID NOs: 56 to 58, 76 to 78 and 81 to 83 and (2) sequences in which up to 4 amino acids in any of SEQ ID NOs: 56 to 58, 76 to 78 and 81 to 83 are substituted by another amino acid. In one embodiment, said ankyrin repeat module comprises an amino acid sequence selected from the group consisting of (1) SEQ ID NOs: 56 to 58, 76 to 78 and 81 to 83 and (2) sequences in which up to 3 amino acids in any of SEQ ID NOs: 56 to 58, 76 to 78 and 81 to 83 are substituted by another amino acid. In one embodiment, said ankyrin repeat module comprises an amino acid sequence selected from the group consisting of (1) SEQ ID NOs: 56 to 58, 76 to 78 and 81 to 83 and (2) sequences in which up to 2 amino acids in any of SEQ ID NOs: 56 to 58, 76 to 78 and 81 to 83 are substituted by another amino acid. In one embodiment, said ankyrin repeat module comprises an amino acid sequence selected from the group consisting of (1) SEQ ID NOs: 56 to 58, 76 to 78 and 81 to 83 and (2) sequences in which up to 1 amino acid in any of SEQ ID NOs: 56 to 58, 76 to 78 and 81 to 83 is substituted by another amino acid. In one embodiment, all of said 10, 9, 8, 7, 6, 5, 4, 3, 2, or 1 amino acid substitutions occur in framework positions of said ankyrin repeat module(s). In one embodiment, said ankyrin repeat module comprises an amino acid sequence selected from the group consisting of SEQ ID NOs: 56 to 58, 76 to 78 and 81 to 83.

In one embodiment, the recombinant binding protein of the invention comprises an ankyrin repeat domain, wherein said ankyrin repeat domain has binding specificity for CD40, and wherein said ankyrin repeat domain comprises an ankyrin repeat module comprising an amino acid sequence selected from the group consisting of (1) SEQ ID NOs: 76, 77, and 78 and (2) sequences in which up to 10, or up to 9, or up to 8, or up to 7, or up to 6, or up to 5, or up to 4, or up to 3, or up to 2, or up to 1 amino acids in any of SEQ ID NOs: 76, 77, and 78 are substituted by another amino acid. Thus, in one embodiment, said ankyrin repeat module comprises an amino acid sequence selected from the group consisting of (1) SEQ ID NOs: 76, 77, and 78 and (2) sequences in which up to 10 amino acids in any of SEQ ID NOs: 76, 77, and 78 are substituted by another amino acid. In one embodiment, said ankyrin repeat module comprises an amino acid sequence selected from the group consisting of (1) SEQ ID NOs: 76, 77, and 78and (2) sequences in which up to 9 amino acids in any of SEQ ID NOs: 76, 77, and 78 are substituted by another amino acid. In one embodiment, said ankyrin repeat module comprises an amino acid sequence selected from the group consisting of (1) SEQ ID NOs: 76, 77, and 78 and (2) sequences in which up to 8 amino acids in any of SEQ ID NOs: 76, 77, and 78 are substituted by another amino acid. In one embodiment, said ankyrin repeat module comprises an amino acid sequence selected from the group consisting of (1) SEQ ID NOs: 76, 77, and 78 and (2) sequences in which up to 7 amino acids in any of SEQ ID NOs: 76, 77, and 78 are substituted by another amino acid. In one embodiment, said ankyrin repeat module comprises an amino acid sequence selected from the group consisting of (1) SEQ ID NOs: 76, 77, and 78 and (2) sequences in which up to 6 amino acids in any of SEQ ID NOs: 76, 77, and 78 are substituted by another amino acid. In one embodiment, said ankyrin repeat module comprises an amino acid sequence selected from the group consisting of (1) SEQ ID NOs: 76, 77, and 78 and (2) sequences in which up to 5 amino acids in any of SEQ ID NOs: 76, 77, and 78 are substituted by another amino acid. In one embodiment, said ankyrin repeat module comprises an amino acid sequence selected from the group consisting of (1) SEQ ID NOs: 76, 77, and 78 and (2) sequences in which up to 4 amino acids in any of SEQ ID NOs: 76, 77, and 78 are substituted by another amino acid. In one embodiment, said ankyrin repeat module comprises an amino acid sequence selected from the group consisting of (1) SEQ ID NOs: 76, 77, and 78 and (2) sequences in which up to 3 amino acids in any of SEQ ID NOs: 76, 77, and 78 are substituted by another amino acid. In one embodiment, said ankyrin repeat module comprises an amino acid sequence selected from the group consisting of (1) SEQ ID NOs: 76, 77, and 78 and (2) sequences in which up to 2 amino acids in any of SEQ ID NOs: 76, 77, and 78 are substituted by another amino acid. In one embodiment, said ankyrin repeat module comprises an amino acid sequence selected from the group consisting of (1) SEQ ID NOs: 76, 77, and 78 and (2) sequences in which up to 1 amino acid in any of SEQ ID NOs: 76, 77, and 78 is substituted by another amino acid. In one embodiment, all of said 10, 9, 8, 7, 6, 5, 4, 3, 2, or 1 amino acid substitutions occur in framework positions of said ankyrin repeat module(s). In one embodiment, said ankyrin repeat module comprises an amino acid sequence selected from the group consisting of SEQ ID NOs: 76, 77, and 78.

In one embodiment, the recombinant binding protein of the invention comprises an ankyrin repeat domain, wherein said ankyrin repeat domain has binding specificity for CD40, and wherein said ankyrin repeat domain comprises an ankyrin repeat module comprising the amino acid sequence of SEQ ID NO: 56 or a sequence in which one or two amino acids in SEQ ID NO: 56 are substituted by another amino acid. In one embodiment, said ankyrin repeat module comprises the amino acid sequence of SEQ ID NO: 57 or a sequence in which one or two amino acids in SEQ ID NO: 57 are substituted by another amino acid. In one embodiment, said ankyrin repeat module comprises the amino acid sequence of SEQ ID NO: 58 or a sequence in which one or two amino acids in SEQ ID NO: 58 are substituted by another amino acid. In one embodiment, said ankyrin repeat module comprises the amino acid sequence of SEQ ID NO: 76 or a sequence in which one or two amino acids in SEQ ID NO: 76 are substituted by another amino acid. In one embodiment, said ankyrin repeat module comprises the amino acid sequence of SEQ ID NO: 77 or a sequence in which one or two amino acids in SEQ ID NO: 77 are substituted by another amino acid. In one embodiment, said ankyrin repeat module comprises the amino acid sequence of SEQ ID NO: 78 or a sequence in which one or two amino acids in SEQ ID NO: 78 are substituted by another amino acid. In one embodiment, said ankyrin repeat module comprises the amino acid sequence of SEQ ID NO: 81 or a sequence in which one or two amino acids in SEQ ID NO: 81 are substituted by another amino acid. In one embodiment, said ankyrin repeat module comprises the amino acid sequence of SEQ ID NO: 82 or a sequence in which one or two amino acids in SEQ ID NO: 82 are substituted by another amino acid. In one embodiment, said ankyrin repeat module comprises the amino acid sequence of SEQ ID NO: 83 or a sequence in which one or two amino acids in SEQ ID NO: 83 are substituted by another amino acid. In one embodiment, said ankyrin repeat module comprises the amino acid sequence of SEQ ID NO: 56. In one embodiment, said ankyrin repeat module comprises the amino acid sequence of SEQ ID NO: 57. In one embodiment, said ankyrin repeat module comprises the amino acid sequence of SEQ ID NO: 58. In one embodiment, said ankyrin repeat module comprises the amino acid sequence of SEQ ID NO: 76. In one embodiment, said ankyrin repeat module comprises the amino acid sequence of SEQ ID NO: 77. In one embodiment, said ankyrin repeat module comprises the amino acid sequence of SEQ ID NO: 78. In one embodiment, said ankyrin repeat module comprises the amino acid sequence of SEQ ID NO: 81. In one embodiment, said ankyrin repeat module comprises the amino acid sequence of SEQ ID NO: 82. In one embodiment, said ankyrin repeat module comprises the amino acid sequence of SEQ ID NO: 83.

In one preferred embodiment, all of said amino acid substitutions of said ankyrin repeat module(s) as described and referred to herein occur in framework positions of said ankyrin repeat module(s), wherein typically the overall structure of the module(s) is not affected by the substitutions. In one more preferred embodiment, all of said amino acid substitutions of said ankyrin repeat module(s) as described and referred to herein occur in positions other than the randomized positions 3, 4, 6, 14 and 15 of said ankyrin repeat module(s) of SEQ ID NOs: 39 to 95.

In one embodiment, the ankyrin repeat domain of the invention comprises a first ankyrin repeat module and a second ankyrin repeat module. In one embodiment, said first ankyrin repeat module is located N-terminally of said second ankyrin repeat module within said ankyrin repeat domain.

In one embodiment, the ankyrin repeat domain of the invention comprises a first ankyrin repeat module and a second ankyrin repeat module and a third ankyrin repeat module. In one preferred embodiment, said first ankyrin repeat module is located N-terminally of said second ankyrin repeat module within said ankyrin repeat domain, and said second ankyrin repeat module is located N-terminally of said third ankyrin repeat module within said ankyrin repeat domain.

In one embodiment, said first, said second and, if present, said third ankyrin repeat module comprises an amino acid sequence selected from the group consisting of (1) SEQ ID NOs: 39 to 95 and (2) sequences in which up to 10, or up to 9, or up to 8, or up to 7, or up to 6, or up to 5, or up to 4, or up to 3, or up to 2, or up to 1 amino acids in any of SEQ ID NOs: 39 to 95 are substituted by another amino acid. In one embodiment, said first, said second and, if present, said third ankyrin repeat module comprises an amino acid sequence selected from the group consisting of (1) SEQ ID NOs: 56 to 58, 76 to 78 and 81 to 83 and (2) sequences in which up to 10, or up to 9, or up to 8, or up to 7, or up to 6, or up to 5, or up to 4, or up to 3, or up to 2, or up to 1 amino acids in any of SEQ ID NOs: 56 to 58, 76 to 78 and 81 to 83 are substituted by another amino acid. In one embodiment, said first, said second and, if present, said third ankyrin repeat module comprises an amino acid sequence selected from the group consisting of (1) SEQ ID NOs: 76, 77, and 78 and (2) sequences in which up to 10, or up to 9, or up to 8, or up to 7, or up to 6, or up to 5, or up to 4, or up to 3, or up to 2, or up to 1 amino acids in any of SEQ ID NOs: 76, 77, and 78 are substituted by another amino acid.

In one embodiment, the ankyrin repeat domain of the invention comprises a first ankyrin repeat module and a second ankyrin repeat module and a third ankyrin repeat module, wherein said first ankyrin repeat module comprises an amino acid sequence selected from the group consisting of (1) SEQ ID NO: 56 and (2) sequences in which up to 10 amino acids in SEQ ID NO: 56 are substituted by another amino acid, and said second ankyrin repeat module comprises an amino acid sequence selected from the group consisting of (1) SEQ ID NO: 57 and (2) sequences in which up to 10 amino acids of SEQ ID NO: 57 are substituted by another amino acid, and said third ankyrin repeat module comprises an amino acid sequence selected from the group consisting of (1) SEQ ID NO: 58 and (2) sequences in which up to 10 amino acids of SEQ ID NO: 58 are substituted by another amino acid. In one embodiment, in such an ankyrin repeat domain said first ankyrin repeat module comprises an amino acid sequence selected from the group consisting of (1) SEQ ID NO: 56 and (2) sequences in which up to 6 amino acids in SEQ ID NO: 56 are substituted by another amino acid, and said second ankyrin repeat module comprises an amino acid sequence selected from the group consisting of (1) SEQ ID NO: 57 and (2) sequences in which up to 6 amino acids of SEQ ID NO: 57 are substituted by another amino acid, and said third ankyrin repeat module comprises an amino acid sequence selected from the group consisting of (1) SEQ ID NO: 58 and (2) sequences in which up to 6 amino acids of SEQ ID NO: 58 are substituted by another amino acid. In one embodiment, in such an ankyrin repeat domain said first ankyrin repeat module comprises an amino acid sequence selected from the group consisting of (1) SEQ ID NO: 56 and (2) sequences in which up to 3 amino acids in SEQ ID NO: 56 are substituted by another amino acid, and said second ankyrin repeat module comprises an amino acid sequence selected from the group consisting of (1) SEQ ID NO: 57 and (2) sequences in which up to 3 amino acids of SEQ ID NO: 57 are substituted by another amino acid, and said third ankyrin repeat module comprises an amino acid sequence selected from the group consisting of (1) SEQ ID NO: 58 and (2) sequences in which up to 3 amino acids of SEQ ID NO: 58 are substituted by another amino acid. In one embodiment, in such an ankyrin repeat domain said first ankyrin repeat module comprises an amino acid sequence selected from the group consisting of (1) SEQ ID NO: 56 and (2) sequences in which up to 2 amino acids in SEQ ID NO: 56 are substituted by another amino acid, and said second ankyrin repeat module comprises an amino acid sequence selected from the group consisting of (1) SEQ ID NO: 57 and (2) sequences in which up to 2 amino acids of SEQ ID NO: 57 are substituted by another amino acid, and said third ankyrin repeat module comprises an amino acid sequence selected from the group consisting of (1) SEQ ID NO: 58 and (2) sequences in which up to 2 amino acids of SEQ ID NO: 58 are substituted by another amino acid. In one embodiment, in such an ankyrin repeat domain said first ankyrin repeat module comprises an amino acid sequence selected from the group consisting of (1) SEQ ID NO: 56 and (2) sequences in which 1 amino acid in SEQ ID NO: 56 is substituted by another amino acid, and said second ankyrin repeat module comprises an amino acid sequence selected from the group consisting of (1) SEQ ID NO: 57 and (2) sequences in which 1 amino acid of SEQ ID NO: 57 is substituted by another amino acid, and said third ankyrin repeat module comprises an amino acid sequence selected from the group consisting of (1) SEQ ID NO: 58 and (2) sequences in which 1 amino acid of SEQ ID NO: 58 is substituted by another amino acid. In one embodiment, in such an ankyrin repeat domain said first ankyrin repeat module comprises the amino acid sequence of SEQ ID NO: 56, and said second ankyrin repeat module comprises the amino acid sequence of SEQ ID NO: 57, and said third ankyrin repeat module comprises the amino acid sequence of SEQ ID NO: 58. In one preferred embodiment, all of said amino acid substitutions of said ankyrin repeat module(s) occur in framework positions other than the randomized positions 3, 4, 6, 14 and 15 of said ankyrin repeat module(s) of SEQ ID NOs: 56, 57, and 58, wherein typically the overall structure of the module(s) is not affected by the substitutions. In one preferred embodiment, said first ankyrin repeat module is located N-terminally of said second ankyrin repeat module within said ankyrin repeat domain, and said second ankyrin repeat module is located N-terminally of said third ankyrin repeat module within said ankyrin repeat domain.

In one embodiment, the ankyrin repeat domain of the invention comprises a first ankyrin repeat module and a second ankyrin repeat module and a third ankyrin repeat module, wherein said first ankyrin repeat module comprises an amino acid sequence selected from the group consisting of (1) SEQ ID NO: 76 and (2) sequences in which up to 10 amino acids in SEQ ID NO: 76 are substituted by another amino acid, and said second ankyrin repeat module comprises an amino acid sequence selected from the group consisting of (1) SEQ ID NO: 77 and (2) sequences in which up to 10 amino acids of SEQ ID NO: 77 are substituted by another amino acid, and said third ankyrin repeat module comprises an amino acid sequence selected from the group consisting of (1) SEQ ID NO: 78 and (2) sequences in which up to 10 amino acids of SEQ ID NO: 78 are substituted by another amino acid. In one embodiment, in such an ankyrin repeat domain said first ankyrin repeat module comprises an amino acid sequence selected from the group consisting of (1) SEQ ID NO: 76 and (2) sequences in which up to 6 amino acids in SEQ ID NO: 76 are substituted by another amino acid, and said second ankyrin repeat module comprises an amino acid sequence selected from the group consisting of (1) SEQ ID NO: 77 and (2) sequences in which up to 6 amino acids of SEQ ID NO: 77 are substituted by another amino acid, and said third ankyrin repeat module comprises an amino acid sequence selected from the group consisting of (1) SEQ ID NO: 78 and (2) sequences in which up to 6 amino acids of SEQ ID NO: 78 are substituted by another amino acid. In one embodiment, in such an ankyrin repeat domain said first ankyrin repeat module comprises an amino acid sequence selected from the group consisting of (1) SEQ ID NO: 76 and (2) sequences in which up to 3 amino acids in SEQ ID NO: 76 are substituted by another amino acid, and said second ankyrin repeat module comprises an amino acid sequence selected from the group consisting of (1) SEQ ID NO: 77 and (2) sequences in which up to 3 amino acids of SEQ ID NO: 77 are substituted by another amino acid, and said third ankyrin repeat module comprises an amino acid sequence selected from the group consisting of (1) SEQ ID NO: 78 and (2) sequences in which up to 3 amino acids of SEQ ID NO: 78 are substituted by another amino acid. In one embodiment, in such an ankyrin repeat domain said first ankyrin repeat module comprises an amino acid sequence selected from the group consisting of (1) SEQ ID NO: 76 and (2) sequences in which up to 2 amino acids in SEQ ID NO: 76 are substituted by another amino acid, and said second ankyrin repeat module comprises an amino acid sequence selected from the group consisting of (1) SEQ ID NO: 77 and (2) sequences in which up to 2 amino acids of SEQ ID NO: 77 are substituted by another amino acid, and said third ankyrin repeat module comprises an amino acid sequence selected from the group consisting of (1) SEQ ID NO: 78 and (2) sequences in which up to 2 amino acids of SEQ ID NO: 78 are substituted by another amino acid. In one embodiment, in such an ankyrin repeat domain said first ankyrin repeat module comprises an amino acid sequence selected from the group consisting of (1) SEQ ID NO: 76 and (2) sequences in which 1 amino acid in SEQ ID NO: 76 is substituted by another amino acid, and said second ankyrin repeat module comprises an amino acid sequence selected from the group consisting of (1) SEQ ID NO: 77 and (2) sequences in which 1 amino acid of SEQ ID NO: 77 is substituted by another amino acid, and said third ankyrin repeat module comprises an amino acid sequence selected from the group consisting of (1) SEQ ID NO: 78 and (2) sequences in which 1 amino acid of SEQ ID NO: 78 is substituted by another amino acid. In one embodiment, in such an ankyrin repeat domain said first ankyrin repeat module comprises the amino acid sequence of SEQ ID NO: 76, and said second ankyrin repeat module comprises the amino acid sequence of SEQ ID NO: 77, and said third ankyrin repeat module comprises the amino acid sequence of SEQ ID NO: 78. In one preferred embodiment, all of said amino acid substitutions of said ankyrin repeat module(s) occur in framework positions other than the randomized positions 3, 4, 6, 14 and 15 of said ankyrin repeat module(s) of SEQ ID NOs: 76, 77, and 78, wherein typically the overall structure of the module(s) is not affected by the substitutions. In one preferred embodiment, said first ankyrin repeat module is located N-terminally of said second ankyrin repeat module, and said second ankyrin repeat module is located N-terminally of said third ankyrin repeat module within said ankyrin repeat domain.

In one embodiment, the ankyrin repeat domain of the invention comprises a first ankyrin repeat module and a second ankyrin repeat module and a third ankyrin repeat module, wherein said first ankyrin repeat module comprises an amino acid sequence selected from the group consisting of (1) SEQ ID NO: 81 and (2) sequences in which up to 10 amino acids in SEQ ID NO: 81 are substituted by another amino acid, and said second ankyrin repeat module comprises an amino acid sequence selected from the group consisting of (1) SEQ ID NO: 82 and (2) sequences in which up to 10 amino acids of SEQ ID NO: 82 are substituted by another amino acid, and said third ankyrin repeat module comprises an amino acid sequence selected from the group consisting of (1) SEQ ID NO: 83 and (2) sequences in which up to 10 amino acids of SEQ ID NO: 83 are substituted by another amino acid. In one embodiment, in such an ankyrin repeat domain said first ankyrin repeat module comprises an amino acid sequence selected from the group consisting of (1) SEQ ID NO: 81 and (2) sequences in which up to 6 amino acids in SEQ ID NO: 81 are substituted by another amino acid, and said second ankyrin repeat module comprises an amino acid sequence selected from the group consisting of (1) SEQ ID NO: 82 and (2) sequences in which up to 6 amino acids of SEQ ID NO: 82 are substituted by another amino acid, and said third ankyrin repeat module comprises an amino acid sequence selected from the group consisting of (1) SEQ ID NO: 83 and (2) sequences in which up to 6 amino acids of SEQ ID NO: 83 are substituted by another amino acid. In one embodiment, in such an ankyrin repeat domain said first ankyrin repeat module comprises an amino acid sequence selected from the group consisting of (1) SEQ ID NO: 81 and (2) sequences in which up to 3 amino acids in SEQ ID NO: 81 are substituted by another amino acid, and said second ankyrin repeat module comprises an amino acid sequence selected from the group consisting of (1) SEQ ID NO: 82 and (2) sequences in which up to 3 amino acids of SEQ ID NO: 82 are substituted by another amino acid, and said third ankyrin repeat module comprises an amino acid sequence selected from the group consisting of (1) SEQ ID NO: 83 and (2) sequences in which up to 3 amino acids of SEQ ID NO: 83 are substituted by another amino acid. In one embodiment, in such an ankyrin repeat domain said first ankyrin repeat module comprises an amino acid sequence selected from the group consisting of (1) SEQ ID NO: 81 and (2) sequences in which up to 2 amino acids in SEQ ID NO: 81 are substituted by another amino acid, and said second ankyrin repeat module comprises an amino acid sequence selected from the group consisting of (1) SEQ ID NO: 82 and (2) sequences in which up to 2 amino acids of SEQ ID NO: 82 are substituted by another amino acid, and said third ankyrin repeat module comprises an amino acid sequence selected from the group consisting of (1) SEQ ID NO: 83 and (2) sequences in which up to 2 amino acids of SEQ ID NO: 83 are substituted by another amino acid. In one embodiment, in such an ankyrin repeat domain said first ankyrin repeat module comprises an amino acid sequence selected from the group consisting of (1) SEQ ID NO: 81 and (2) sequences in which 1 amino acid in SEQ ID NO: 81 is substituted by another amino acid, and said second ankyrin repeat module comprises an amino acid sequence selected from the group consisting of (1) SEQ ID NO: 82 and (2) sequences in which 1 amino acid of SEQ ID NO: 82 is substituted by another amino acid, and said third ankyrin repeat module comprises an amino acid sequence selected from the group consisting of (1) SEQ ID NO: 83 and (2) sequences in which 1 amino acid of SEQ ID NO: 83 is substituted by another amino acid. In one embodiment, in such an ankyrin repeat domain said first ankyrin repeat module comprises the amino acid sequence of SEQ ID NO: 81, and said second ankyrin repeat module comprises the amino acid sequence of SEQ ID NO: 82, and said third ankyrin repeat module comprises the amino acid sequence of SEQ ID NO: 83. In one preferred embodiment, all of said amino acid substitutions of said ankyrin repeat module(s) occur in framework positions other than the randomized positions 3, 4, 6, 14 and 15 of said ankyrin repeat module(s) of SEQ ID NOs: 81, 82, and 83, wherein typically the overall structure of the module(s) is not affected by the substitutions. In one preferred embodiment, said first ankyrin repeat module is located N-terminally of said second ankyrin repeat module within said ankyrin repeat domain, and said second ankyrin repeat module is located N-terminally of said third ankyrin repeat module within said ankyrin repeat domain.

In one preferred embodiment, the ankyrin repeat domain of the invention as described in any of the embodiments herein, further comprises an N-terminal capping module and/or a C-terminal capping module. In one more preferred embodiment, the ankyrin repeat domain of the invention comprises from N-terminus to C-terminus: an N-terminal capping module; one, two, three or more ankyrin repeat module(s) as described more specifically in any of the embodiments herein; and a C-terminal capping module.

In one embodiment, said N-terminal capping module comprises an amino acid sequence selected from the group consisting of (1) SEQ ID NOs: 5 to 8 and (2) sequences in which up to 10, or up to 9, or up to 8, or up to 7, or up to 6, or up to 5, or up to 4, or up to 3, or up to 2, or up to 1 amino acids in any of SEQ ID NOs: 5 to 7 are substituted by other amino acids. Thus, in one embodiment, said N-terminal capping module comprises an amino acid sequence selected from the group consisting of (1) SEQ ID NOs: 5 to 8 and (2) sequences in which up to 10 amino acids in any of SEQ ID NOs: 5 to 7 are substituted by another amino acid. In one embodiment, said N-terminal capping module comprises an amino acid sequence selected from the group consisting of (1) SEQ ID NOs: 5 to 8 and (2) sequences in which up to 9 amino acids in any of SEQ ID NOs: 5 to 7 are substituted by another amino acid. In one embodiment, said N-terminal capping module comprises an amino acid sequence selected from the group consisting of (1) SEQ ID NOs: 5 to 8 and (2) sequences in which up to 8 amino acids in any of SEQ ID NOs: 5 to 7 are substituted by another amino acid. In one embodiment, said N-terminal capping module comprises an amino acid sequence selected from the group consisting of (1) SEQ ID NOs: 5 to 8 and (2) sequences in which up to 7 amino acids in any of SEQ ID NOs: 5 to 7 are substituted by another amino acid. In one embodiment, said N-terminal capping module comprises an amino acid sequence selected from the group consisting of (1) SEQ ID NOs: 5 to 8 and (2) sequences in which up to 6 amino acids in any of SEQ ID NOs: 5 to 7 are substituted by another amino acid. In one embodiment, said N-terminal capping module comprises an amino acid sequence selected from the group consisting of (1) SEQ ID NOs: 5 to 8 and (2) sequences in which up to 5 amino acids in any of SEQ ID NOs: 5 to 7 are substituted by another amino acid. In one embodiment, said N-terminal capping module comprises an amino acid sequence selected from the group consisting of (1) SEQ ID NOs: 5 to 8 and (2) sequences in which up to 4 amino acids in any of SEQ ID NOs: 5 to 7 are substituted by another amino acid. In one embodiment, said N-terminal capping module comprises an amino acid sequence selected from the group consisting of (1) SEQ ID NOs: 5 to 8 and (2) sequences in which up to 3 amino acids in any of SEQ ID NOs: 5 to 7 are substituted by another amino acid. In one embodiment, said N-terminal capping module comprises an amino acid sequence selected from the group consisting of (1) SEQ ID NOs: 5 to 8 and (2) sequences in which up to 2 amino acids in any of SEQ ID NOs: 5 to 7 are substituted by another amino acid. In one embodiment, said N-terminal capping module comprises an amino acid sequence selected from the group consisting of (1) SEQ ID NOs: 5 to 8 and (2) sequences in which up to 1 amino acid in any of SEQ ID NOs: 5 to 7 are substituted by another amino acid. In one preferred embodiment, all of said amino acid substitutions of said N-terminal capping module occur in positions other than position 10 and position 17 of SEQ ID NOs: 5 to 7. In one embodiment, said N-terminal capping module comprises or consists of the amino acid sequence of SEQ ID NO: 5. In one embodiment, said N-terminal capping module comprises or consists of the amino acid sequence of SEQ ID NO: 6. In one embodiment, said N-terminal capping module comprises or consists of the amino acid sequence of SEQ ID NO: 7. In one embodiment, said N-terminal capping module comprises or consists of the amino acid sequence of SEQ ID NO: 8, wherein X represents any amino acid. In one embodiment, G at position 1 and/or S at position 2 of said N-terminal capping module of SEQ ID NOs: 5 to 8 are optionally missing.

In one embodiment, said C-terminal capping module comprises an amino acid sequence selected from the group consisting of (1) SEQ ID NOs: 12 to 15 and (2) sequences in which up to 10, or up to 9, or up to 8, or up to 7, or up to 6, or up to 5, or up to 4, or up to 3, or up to 2, or up to 1 amino acids in any of SEQ ID NOs: 12 to 14 are substituted by other amino acids. Thus, in one embodiment, said C-terminal capping module comprises an amino acid sequence selected from the group consisting of (1) SEQ ID NOs: 12 to 15 and (2) sequences in which up to 10 amino acids in any of SEQ ID NOs: 12 to 14 are substituted by another amino acid. In one embodiment, said C-terminal capping module comprises an amino acid sequence selected from the group consisting of (1) SEQ ID NOs: 12 to 15 and (2) sequences in which up to 9 amino acids in any of SEQ ID NOs: 12 to 14 are substituted by another amino acid. In one embodiment, said C-terminal capping module comprises an amino acid sequence selected from the group consisting of (1) SEQ ID NOs: 12 to 15 and (2) sequences in which up to 8 amino acids in any of SEQ ID NOs: 12 to 14 are substituted by another amino acid. In one embodiment, said C-terminal capping module comprises an amino acid sequence selected from the group consisting of (1) SEQ ID NOs: 12 to 15 and (2) sequences in which up to 7 amino acids in any of SEQ ID NOs: 12 to 14 are substituted by another amino acid. In one embodiment, said C-terminal capping module comprises an amino acid sequence selected from the group consisting of (1) SEQ ID NOs: 12 to 15 and (2) sequences in which up to 6 amino acids in any of SEQ ID NOs: 12 to 14 are substituted by another amino acid. In one embodiment, said C-terminal capping module comprises an amino acid sequence selected from the group consisting of (1) SEQ ID NOs: 12 to 15 and (2) sequences in which up to 5 amino acids in any of SEQ ID NOs: 12 to 14 are substituted by another amino acid. In one embodiment, said C-terminal capping module comprises an amino acid sequence selected from the group consisting of (1) SEQ ID NOs: 12 to 15 and (2) sequences in which up to 4 amino acids in any of SEQ ID NOs: 12 to 14 are substituted by another amino acid. In one embodiment, said C-terminal capping module comprises an amino acid sequence selected from the group consisting of (1) SEQ ID NOs: 12 to 15 and (2) sequences in which up to 3 amino acids in any of SEQ ID NOs: 12 to 14 are substituted by another amino acid. In one embodiment, said C-terminal capping module comprises an amino acid sequence selected from the group consisting of (1) SEQ ID NOs: 12 to 15 and (2) sequences in which up to 2 amino acids in any of SEQ ID NOs: 12 to 14 are substituted by another amino acid. In one embodiment, said C-terminal capping module comprises an amino acid sequence selected from the group consisting of (1) SEQ ID NOs: 12 to 15 and (2) sequences in which up to 1 amino acid in any of SEQ ID NOs: 12 to 14 are substituted by another amino acid. In one preferred embodiment, all of said amino acid substitutions of said C-terminal capping module occur in positions other than position 14 and position 18 of SEQ ID NOs: 13 to 14. In one embodiment, said C-terminal capping module comprises or consists of the amino acid sequence of SEQ ID NO: 12. In one embodiment, said C-terminal capping module comprises or consists of the amino acid sequence of SEQ ID NO: 13. In one embodiment, said C-terminal capping module comprises or consists of the amino acid sequence of SEQ ID NO: 14. In one embodiment, said C-terminal capping module comprises or consists of the amino acid sequence of SEQ ID NO: 15, wherein X represents any amino acid. In one embodiment, L at the second last position and/or N at the last position of SEQ ID NO: 12 are optionally substituted by A.

In one preferred embodiment, the designed ankyrin repeat domain provided by the present invention and described herein comprises sequence modifications in the N-terminal capping module and/or in the C-terminal capping module that lead to improved pharmacokinetic properties of said designed ankyrin repeat domain compared to the designed ankyrin repeat domain not comprising said sequence modifications.

In one more preferred embodiment, the designed ankyrin repeat domain of the invention comprises an N-terminal capping module with improved pharmacokinetic properties, wherein said N-terminal capping module has an amino acid sequence wherein the amino acid at position 8 is Q and/or the amino acid at position 15 is L. Examples of such N-terminal capping modules are provided in SEQ ID NOs: 5 to 8. In one embodiment, said N-terminal capping module has an amino acid sequence wherein the amino acid at position 4 is S, the amino acid at position 8 is Q, the amino acid at position 15 is L, the amino acid at position 17 is T, the amino acid at position 20 is T, and/or the amino acid at position 23 is Q. An example of such an N-terminal capping module is provided in SEQ ID NO: 7. In one preferred embodiment, said N-terminal capping module comprises an amino acid sequence of 30 amino acids. In a further preferred embodiment, said N-terminal capping module consists of an amino acid sequence of 30 amino acids. Preferably, said position numbers of positions of the N-terminal capping module are determined by alignment to SEQ ID NO: 5 using the position numbers of SEQ ID NO: 5. Preferably, said alignment comprises no amino acid gaps. Sequence alignment generation is a procedure well known in the art.

In another more preferred embodiment, the designed ankyrin repeat domain of the invention comprises a C-terminal capping module with improved pharmacokinetic properties, wherein said C-terminal capping module has an amino acid sequence wherein the amino acid at position 14 is R and/or the amino acid at position 18 is Q. Examples of such C-terminal capping modules are provided in SEQ ID NOs: 13 to 15. In one embodiment, said C-terminal capping module has an amino acid sequence wherein the amino acid at position 3 is T, the amino acid at position 4 is Q, the amino acid at position 6 is T, the amino acid at position 14 is R, the amino acid at position 18 is Q, the amino acid at position 19 is Q, the amino acid at position 22 is S, and/or the amino acid at position 26 is Q. An example of such an N-terminal capping module is provided in SEQ ID NO: 14. In a preferred embodiment, said C-terminal capping module comprises an amino acid sequence of 28 amino acids. In a further preferred embodiment, said C-terminal capping module consists of an amino acid sequence of 28 amino acids. Preferably, said position numbers of positions of the C-terminal capping module are determined by alignment to SEQ ID NO: 13 using the position numbers of SEQ ID NO: 13. Preferably, said alignment comprises no amino acid gaps.

In one embodiment, the term improved pharmacokinetic properties refers to an increased area under the curve, a reduced clearance, or an increased terminal half-life. In one embodiment, the term improved pharmacokinetic properties refers to an increased area under the curve. In one embodiment, said increase in area under the curve is at least 5%, 10%, 15%, 20%, 25%, 30%, 35%, 40%, 45%, 50%, 55%, 60%, 65%, 70%, 75%, 80%, most preferably 85%. In one embodiment, the term improved pharmacokinetic properties refers to a reduced clearance. In one embodiment, said reduction in clearance is at least 5%, 10%, 15%, 20%, 25%, 30%, 35%, 40%, most preferably 45%. In one embodiment, the term improved pharmacokinetic properties refers to an increased terminal half-life. In one embodiment, said increase in terminal half-life is at least 5%, 10%, 15%, 20%, 25%, 30%, 35%, 40%, 45%, 50%, 55%, 60%, 65%, 70%, 75%, 80%, most preferably 85%. In one embodiment the pharmacokinetic parameters are determined in mouse. Preferably, said pharmacokinetic parameters in mouse are determined by applying a protein at a dose of 1 mg/kg by intravenous injection into the tail vein of Balb/c mice. In one embodiment the pharmacokinetic parameters are determined in cynomolgus monkey. Preferably, said pharmacokinetic parameters in cynomolgus monkey are determined by applying a protein at a dose of 1 mg/kg by 30 min intravenous injection.

In a preferred embodiment, the designed ankyrin repeat domain provided by the present invention and described herein specifically binds to the N-terminal cysteine rich domain (CRD) 1 of the CD40 receptor. The amino acid sequence of the CD40 receptor is herein provided in SEQ ID NO: 96, where the amino acid residues 23-59 form CRD1, amino acid residues 62-103 form CRD2, amino acid residues 105-143 form CRD3 and amino acid residues 146-186 form CRD4. Thus, in a preferred embodiment, the CD40 epitope to which the ankyrin repeat domain having binding specificity for CD40 binds to is located in CRD1, i.e., within the amino acid residues 23 to 59 of the CD40 receptor (SEQ ID NO: 96). In another aspect, the invention provides a recombinant binding protein comprising an ankyrin repeat domain, wherein said ankyrin repeat domain has binding specificity for CD40, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with any one of SEQ ID NOs: 16 to 35, wherein G at position 1 and/or S at position 2 of SEQ ID NOs: 16 to 35 are optionally missing. Thus, in one embodiment, said ankyrin repeat domain comprises an amino acid sequence with at least 80% amino acid sequence identity with any one of SEQ ID NOs: 16 to 35, wherein G at position 1 and/or S at position 2 of SEQ ID NOs: 16 to 35 are optionally missing. In one embodiment, said ankyrin repeat domain comprises an amino acid sequence with at least 90% amino acid sequence identity with any one of SEQ ID NOs: 16 to 35, wherein G at position 1 and/or S at position 2 of SEQ ID NOs: 16 to 35 are optionally missing. In one embodiment, said ankyrin repeat domain comprises an amino acid sequence with at least 93% amino acid sequence identity with any one of SEQ ID NOs: 16 to 35, wherein G at position 1 and/or S at position 2 of SEQ ID NOs: 16 to 35 are optionally missing. In one embodiment, said ankyrin repeat domain comprises an amino acid sequence with at least 95% amino acid sequence identity with any one of SEQ ID NOs: 16 to 35, wherein G at position 1 and/or S at position 2 of SEQ ID NOs: 16 to 35 are optionally missing. In one embodiment, said ankyrin repeat domain comprises an amino acid sequence with at least 98% amino acid sequence identity with any one of SEQ ID NOs: 16 to 35, wherein G at position 1 and/or S at position 2 of SEQ ID NOs: 16 to 35 are optionally missing. Thus, in one preferred embodiment, the recombinant binding protein of the invention comprises an ankyrin repeat domain having binding specificity for CD40, wherein said ankyrin repeat domain comprises or consists of an amino acid sequence selected from the group consisting of SEQ ID NOs: 16 to 35, wherein G at position 1 and/or S at position 2 of SEQ ID NOs: 16 to 35 are optionally missing.

In one embodiment, the recombinant binding protein of the invention comprises an ankyrin repeat domain having binding specificity for CD40, wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with any one of SEQ ID NOs: 22, 29, and 31, wherein G at position 1 and/or S at position 2 of SEQ ID NOs: 22, 29, and 31 are optionally missing. Thus, in one embodiment, said ankyrin repeat domain comprises an amino acid sequence with at least 80% amino acid sequence identity with any one of SEQ ID NOs: 22, 29, and 31, wherein G at position 1 and/or S at position 2 of SEQ ID NOs: 22, 29, and 31 are optionally missing. In one embodiment, said ankyrin repeat domain comprises an amino acid sequence with at least 90% amino acid sequence identity with any one of SEQ ID NOs: 22, 29, and 31, wherein G at position 1 and/or S at position 2 of SEQ ID NOs: 22, 29, and 31 are optionally missing. In one embodiment, said ankyrin repeat domain comprises an amino acid sequence with at least 93% amino acid sequence identity with any one of SEQ ID NOs: 22, 29, and 31, wherein G at position 1 and/or S at position 2 of SEQ ID NOs: 22, 29, and 31 are optionally missing. In one embodiment, said ankyrin repeat domain comprises an amino acid sequence with at least 95% amino acid sequence identity with any one of SEQ ID NOs: 22, 29, and 31, wherein G at position 1 and/or S at position 2 of SEQ ID NOs: 22, 29, and 31 are optionally missing. In one embodiment, said ankyrin repeat domain comprises an amino acid sequence with at least 98% amino acid sequence identity with any one of SEQ ID NOs: 22, 29, and 31, wherein G at position 1 and/or S at position 2 of SEQ ID NOs: 22, 29, and 31 are optionally missing. Thus, in one preferred embodiment, the recombinant binding protein of the invention comprises an ankyrin repeat domain having binding specificity for CD40, wherein said ankyrin repeat domain comprises or consists of an amino acid sequence selected from the group consisting of SEQ ID NOs: 22, 29, and 31, wherein G at position 1 and/or S at position 2 of SEQ ID NOs: 22, 29, and 31 are optionally missing.

In one embodiment, the recombinant binding protein of the invention comprises an ankyrin repeat domain having binding specificity for CD40, wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with SEQ ID NO: 29, wherein G at position 1 and/or S at position 2 of SEQ ID NO: 29 are optionally missing. Thus, in one embodiment, said ankyrin repeat domain comprises an amino acid sequence with at least 80% amino acid sequence identity with SEQ ID NO: 29, wherein G at position 1 and/or S at position 2 of SEQ ID NO: 29 are optionally missing. In one embodiment, said ankyrin repeat domain comprises an amino acid sequence with at least 90% amino acid sequence identity with SEQ ID NO: 29, wherein G at position 1 and/or S at position 2 of SEQ ID NO: 29 are optionally missing. In one embodiment, said ankyrin repeat domain comprises an amino acid sequence with at least 93% amino acid sequence identity with SEQ ID NO: 29, wherein G at position 1 and/or S at position 2 of SEQ ID NO: 29 are optionally missing. In one embodiment, said ankyrin repeat domain comprises an amino acid sequence with at least 95% amino acid sequence identity with SEQ ID NO: 29, wherein G at position 1 and/or S at position 2 of SEQ ID NO: 29 are optionally missing. In one embodiment, said ankyrin repeat domain comprises an amino acid sequence with at least 98% amino acid sequence identity with SEQ ID NO: 29, wherein G at position 1 and/or S at position 2 of SEQ ID NO: 29 are optionally missing. Thus, in one preferred embodiment, the recombinant binding protein of the invention comprises an ankyrin repeat domain having binding specificity for CD40, wherein said ankyrin repeat domain comprises or consists of the amino acid sequence of SEQ ID NO: 29, wherein G at position 1 and/or S at position 2 of SEQ ID NO: 29 are optionally missing. In a preferred embodiment, said ankyrin repeat domain having binding specificity for CD40 specifically binds to an epitope in the CRD1 of the CD40 receptor.

In one embodiment, the recombinant binding protein of the invention comprises an ankyrin repeat domain having binding specificity for CD40, wherein said ankyrin repeat domain binds human CD40 in PBS with a dissociation constant (K_{D}) below 10⁻⁷M, or below 7.5×10⁻⁸M, or below 5×10⁻⁸M, or below 2×10⁻⁸M. Thus, in one embodiment, said ankyrin repeat domain binds human CD40 in PBS with a dissociation constant (K_{D}) below 10⁻⁷M. In another embodiment, said ankyrin repeat domain binds human CD40 in PBS with a dissociation constant (K_{D}) below 7.5×10⁻⁸M; and in a further embodiment, said ankyrin repeat domain binds human CD40 in PBS with a dissociation constant (K_{D}) below 5×10⁻⁸M. In one embodiment, said ankyrin repeat domain binds human CD40 in PBS with a dissociation constant (K_{D}) below 2×10⁻⁸M.

In one embodiment, the recombinant binding protein of the invention comprises an ankyrin repeat domain having binding specificity for CD40, wherein said ankyrin repeat domain binds human CD40 in PBS with a dissociation constant (K_{D}) below 10⁻⁷M, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with any one of SEQ ID NOs: 16 to 35, wherein G at position 1 and/or S at position 2 of SEQ ID NOs: 16 to 35 are optionally missing. Thus, in one embodiment, said ankyrin repeat domain comprises an amino acid sequence with at least 80% amino acid sequence identity with any one of SEQ ID NOs: 16 to 35, wherein G at position 1 and/or S at position 2 of SEQ ID NOs: 16 to 35 are optionally missing. In one embodiment, said ankyrin repeat domain comprises an amino acid sequence with at least 90% amino acid sequence identity with any one of SEQ ID NOs: 16 to 35, wherein G at position 1 and/or S at position 2 of SEQ ID NOs: 16 to 35 are optionally missing. In one embodiment, said ankyrin repeat domain comprises an amino acid sequence with at least 93% amino acid sequence identity with any one of SEQ ID NOs: 16 to 35, wherein G at position 1 and/or S at position 2 of SEQ ID NOs: 16 to 35 are optionally missing. In one embodiment, said ankyrin repeat domain comprises an amino acid sequence with at least 95% amino acid sequence identity with any one of SEQ ID NOs: 16 to 35, wherein G at position 1 and/or S at position 2 of SEQ ID NOs: 16 to 35 are optionally missing. In one embodiment, said ankyrin repeat domain comprises an amino acid sequence with at least 98% amino acid sequence identity with any one of SEQ ID NOs: 16 to 35, wherein G at position 1 and/or S at position 2 of SEQ ID NOs: 16 to 35 are optionally missing. Thus, in one embodiment, the recombinant binding protein of the invention comprises an ankyrin repeat domain having binding specificity for CD40, wherein said ankyrin repeat domain binds human CD40 in PBS with a dissociation constant (K_{D}) below 10⁻⁷M, and wherein said ankyrin repeat domain comprises or consists of an amino acid sequence selected from the group consisting of SEQ ID NOs: 16 to 35, wherein G at position 1 and/or S at position 2 of any one of SEQ ID NOs: 16 to 35 are optionally missing.

In one embodiment, the recombinant binding protein of the invention comprises an ankyrin repeat domain having binding specificity for CD40, wherein said ankyrin repeat domain binds human CD40 in PBS with a dissociation constant (K_{D}) below 7.5×10⁻⁸M, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with any one of SEQ ID NOs: 22, 29, and 31, wherein G at position 1 and/or S at position 2 of SEQ ID NOs: 22, 29, and 31 are optionally missing. Thus, in one embodiment, said ankyrin repeat domain comprises an amino acid sequence with at least 80% amino acid sequence identity with any one of SEQ ID NOs: 22, 29, and 31, wherein G at position 1 and/or S at position 2 of SEQ ID NOs: 22, 29, and 31 are optionally missing. In one embodiment, said ankyrin repeat domain comprises an amino acid sequence with at least 90% amino acid sequence identity with any one of SEQ ID NOs: 22, 29, and 31, wherein G at position 1 and/or S at position 2 of SEQ ID NOs: 22, 29, and 31 are optionally missing. In one embodiment, said ankyrin repeat domain comprises an amino acid sequence with at least 93% amino acid sequence identity with any one of SEQ ID NOs: 22, 29, and 31, wherein G at position 1 and/or S at position 2 of SEQ ID NOs: 22, 29, and 31 are optionally missing. In one embodiment, said ankyrin repeat domain comprises an amino acid sequence with at least 95% amino acid sequence identity with any one of SEQ ID NOs: 22, 29, and 31, wherein G at position 1 and/or S at position 2 of SEQ ID NOs: 22, 29, and 31 are optionally missing. In one embodiment, said ankyrin repeat domain comprises an amino acid sequence with at least 98% amino acid sequence identity with any one of SEQ ID NOs: 22, 29, and 31, wherein G at position 1 and/or S at position 2 of SEQ ID NOs: 22, 29, and 31 are optionally missing. Thus, in one embodiment, the recombinant binding protein of the invention comprises an ankyrin repeat domain having binding specificity for CD40, wherein said ankyrin repeat domain binds human CD40 in PBS with a dissociation constant (K_{D}) below 7.5×10⁻⁸M, and wherein said ankyrin repeat domain comprises or consists of an amino acid sequence selected from the group consisting of SEQ ID NOs: 22, 29, and 31, wherein G at position 1 and/or S at position 2 of any one of SEQ ID NOS: 22, 29, and 31 are optionally missing.

In one embodiment, the recombinant binding protein of the invention comprises an ankyrin repeat domain having binding specificity for CD40, wherein said ankyrin repeat domain binds human CD40 in PBS with a dissociation constant (K_{D}) below 5×10⁻⁸M, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with any one of SEQ ID NOs: 22, 29, and 31, wherein G at position 1 and/or S at position 2 of SEQ ID NOs: 22, 29, and 31 are optionally missing. Thus, in one embodiment, said ankyrin repeat domain comprises an amino acid sequence with at least 80% amino acid sequence identity with any one of SEQ ID NOs: 22, 29, and 31, wherein G at position 1 and/or S at position 2 of SEQ ID NOs: 22, 29, and 31 are optionally missing. In one embodiment, said ankyrin repeat domain comprises an amino acid sequence with at least 90% amino acid sequence identity with any one of SEQ ID NOs: 22, 29, and 31, wherein G at position 1 and/or S at position 2 of SEQ ID NOs: 22, 29, and 31 are optionally missing. In one embodiment, said ankyrin repeat domain comprises an amino acid sequence with at least 93% amino acid sequence identity with any one of SEQ ID NOs: 22, 29, and 31, wherein G at position 1 and/or S at position 2 of SEQ ID NOs: 22, 29, and 31 are optionally missing. In one embodiment, said ankyrin repeat domain comprises an amino acid sequence with at least 95% amino acid sequence identity with any one of SEQ ID NOs: 22, 29, and 31, wherein G at position 1 and/or S at position 2 of SEQ ID NOs: 22, 29, and 31 are optionally missing. In one embodiment, said ankyrin repeat domain comprises an amino acid sequence with at least 98% amino acid sequence identity with any one of SEQ ID NOs: 22, 29, and 31, wherein G at position 1 and/or S at position 2 of SEQ ID NOs: 22, 29, and 31 are optionally missing. Thus, in one embodiment, the recombinant binding protein of the invention comprises an ankyrin repeat domain having binding specificity for CD40, wherein said ankyrin repeat domain binds human CD40 in PBS with a dissociation constant (K_{D}) below 5×10⁻⁸M, and wherein said ankyrin repeat domain comprises or consists of an amino acid sequence selected from the group consisting of SEQ ID NOs: 22, 29, and 31, wherein G at position 1 and/or S at position 2 of any one of SEQ ID NOs: 22, 29, and 31 are optionally missing.

In one embodiment, the recombinant binding protein of the invention comprises an ankyrin repeat domain having binding specificity for CD40, wherein said ankyrin repeat domain binds human CD40 in PBS with a dissociation constant (K_{D}) below 10⁻⁷M, or below 7.5×10⁻⁸M, or below 5×10⁻⁸M, or below 2×10⁻⁸M, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with SEQ ID NO: 22, wherein G at position 1 and/or S at position 2 of SEQ ID NO: 22 are optionally missing. Thus, in one embodiment, said ankyrin repeat domain comprises an amino acid sequence with at least 80% amino acid sequence identity with SEQ ID NO: 22, wherein G at position 1 and/or S at position 2 of SEQ ID NO: 22 are optionally missing. In one embodiment, said ankyrin repeat domain comprises an amino acid sequence with at least 90% amino acid sequence identity with SEQ ID NO: 22, wherein G at position 1 and/or S at position 2 of SEQ ID NO: 22 are optionally missing. In one embodiment, said ankyrin repeat domain comprises an amino acid sequence with at least 93% amino acid sequence identity with SEQ ID NO: 22, wherein G at position 1 and/or S at position 2 of SEQ ID NO: 22 are optionally missing. In one embodiment, said ankyrin repeat domain comprises an amino acid sequence with at least 95% amino acid sequence identity with any one of SEQ ID NO: 22, wherein G at position 1 and/or S at position 2 of SEQ ID NO: 22 are optionally missing. In one embodiment, said ankyrin repeat domain comprises an amino acid sequence with at least 98% amino acid sequence identity with any one of SEQ ID NO: 22, wherein G at position 1 and/or S at position 2 of SEQ ID NO: 22 are optionally missing. Thus, in one embodiment, the recombinant binding protein of the invention comprises an ankyrin repeat domain having binding specificity for CD40, wherein said ankyrin repeat domain binds human CD40 in PBS with a dissociation constant (K_{D}) below 10⁻⁷M, or below 7.5×10⁻⁸M, or below 5×10⁻⁸M, or below 2×10−8M, and wherein said ankyrin repeat domain comprises or consists of the amino acid sequence of SEQ ID NO: 22, wherein G at position 1 and/or S at position 2 of any one of SEQ ID NO: 22 are optionally missing.

In one embodiment, the recombinant binding protein of the invention comprises an ankyrin repeat domain having binding specificity for CD40, wherein said ankyrin repeat domain binds human CD40 in PBS with a dissociation constant (K_{D}) below 10⁻⁷M, or below 7.5×10⁻⁸M, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with SEQ ID NO: 29, wherein G at position 1 and/or S at position 2 of SEQ ID NO: 29 are optionally missing. Thus, in one embodiment, said ankyrin repeat domain comprises an amino acid sequence with at least 80% amino acid sequence identity with SEQ ID NO: 29, wherein G at position 1 and/or S at position 2 of SEQ ID NO: 29 are optionally missing. In one embodiment, said ankyrin repeat domain comprises an amino acid sequence with at least 90% amino acid sequence identity with SEQ ID NO: 29, wherein G at position 1 and/or S at position 2 of SEQ ID NO: 29 are optionally missing. In one embodiment, said ankyrin repeat domain comprises an amino acid sequence with at least 93% amino acid sequence identity with SEQ ID NO: 29, wherein G at position 1 and/or S at position 2 of SEQ ID NO: 29 are optionally missing. In one embodiment, said ankyrin repeat domain comprises an amino acid sequence with at least 95% amino acid sequence identity with any one of SEQ ID NO: 29, wherein G at position 1 and/or S at position 2 of SEQ ID NO: 29 are optionally missing. In one embodiment, said ankyrin repeat domain comprises an amino acid sequence with at least 98% amino acid sequence identity with any one of SEQ ID NO: 29, wherein G at position 1 and/or S at position 2 of SEQ ID NO: 29 are optionally missing. Thus, in one embodiment, the recombinant binding protein of the invention comprises an ankyrin repeat domain having binding specificity for CD40, wherein said ankyrin repeat domain binds human CD40 in PBS with a dissociation constant (K_{D}) below 10⁻⁷M, or below 7.5×10⁻⁸M, and wherein said ankyrin repeat domain comprises or consists of the amino acid sequence of SEQ ID NO: 29, wherein G at position 1 and/or S at position 2 of any one of SEQ ID NO: 29 are optionally missing.

In one embodiment, the recombinant binding protein of the invention comprises an ankyrin repeat domain having binding specificity for CD40, wherein said ankyrin repeat domain binds human CD40 in PBS with a dissociation constant (K_{D}) below 10⁻⁷M, or below 7.5×10⁻⁸M, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with SEQ ID NO: 31, wherein G at position 1 and/or S at position 2 of SEQ ID NO: 31 are optionally missing. Thus, in one embodiment, said ankyrin repeat domain comprises an amino acid sequence with at least 80% amino acid sequence identity with SEQ ID NO: 31, wherein G at position 1 and/or S at position 2 of SEQ ID NO: 31 are optionally missing. In one embodiment, said ankyrin repeat domain comprises an amino acid sequence with at least 90% amino acid sequence identity with SEQ ID NO: 31, wherein G at position 1 and/or S at position 2 of SEQ ID NO: 31 are optionally missing. In one embodiment, said ankyrin repeat domain comprises an amino acid sequence with at least 93% amino acid sequence identity with SEQ ID NO: 31, wherein G at position 1 and/or S at position 2 of SEQ ID NO: 31 are optionally missing. In one embodiment, said ankyrin repeat domain comprises an amino acid sequence with at least 95% amino acid sequence identity with any one of SEQ ID NO: 31, wherein G at position 1 and/or S at position 2 of SEQ ID NO: 31 are optionally missing. In one embodiment, said ankyrin repeat domain comprises an amino acid sequence with at least 98% amino acid sequence identity with any one of SEQ ID NO: 31, wherein G at position 1 and/or S at position 2 of SEQ ID NO: 31 are optionally missing. Thus, in one embodiment, the recombinant binding protein of the invention comprises an ankyrin repeat domain having binding specificity for CD40, wherein said ankyrin repeat domain binds human CD40 in PBS with a dissociation constant (K_{D}) below 10⁻⁷M, or below 7.5×10⁻⁸M, and wherein said ankyrin repeat domain comprises or consists of the amino acid sequence of SEQ ID NO: 31, wherein G at position 1 and/or S at position 2 of any one of SEQ ID NO: 31 are optionally missing.

A typical and preferred determination of dissociation constants (K_{D}) of the recombinant binding proteins of the invention with binding specificity for CD40 is by Surface Plasmon Resonance (SPR) analysis. Thus, in one embodiment said binding specificity for CD40 of the recombinant binding proteins of the invention is determined in PBS by Surface Plasmon Resonance (SPR). In one embodiment said binding specificity for CD40 of the recombinant binding proteins of the invention is determined in PBS by Surface Plasmon Resonance (SPR).

In one embodiment, the recombinant binding protein of the invention comprises two or three or more ankyrin repeat domains with binding specificity for CD40. In one preferred embodiment, said recombinant binding protein comprises two or three ankyrin repeat domains with binding specificity for CD40, wherein each of said two or three ankyrin repeat domains independently consists of an ankyrin repeat domain with binding specificity for CD40 as described more specifically in any of the aspects and embodiments herein. Thus, in one particular embodiment, the recombinant binding protein of the invention comprises a first ankyrin repeat domain with binding specificity for CD40 as described more specifically in any of the aspects and embodiments herein, and further comprises a second ankyrin repeat domain with binding specificity for CD40. In one more preferred embodiment, said second ankyrin repeat domain with binding specificity for CD40 is an ankyrin repeat domain with binding specificity for CD40 as described more specifically in any of the aspects and embodiments herein, which may be different or identical to said first ankyrin repeat domain with binding specificity for CD40.

Thus, in one exemplary embodiment, the recombinant binding protein of the invention comprises two ankyrin repeat domains with binding specificity for CD40, wherein each of said ankyrin repeat domains independently comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with SEQ ID NO: 29, wherein G at position 1 and/or S at position 2 of SEQ ID NO: 29 are optionally missing. Thus, in one embodiment, each of said ankyrin repeat domains independently comprises an amino acid sequence with at least 80% amino acid sequence identity with SEQ ID NO: 29, wherein G at position 1 and/or S at position 2 of SEQ ID NO: 29 are optionally missing. In one embodiment, each of said ankyrin repeat domains independently comprises an amino acid sequence with at least 90% amino acid sequence identity with SEQ ID NO: 29, wherein G at position 1 and/or S at position 2 of SEQ ID NO: 29 are optionally missing. In one embodiment, each of said ankyrin repeat domains independently comprises an amino acid sequence with at least 93% amino acid sequence identity with SEQ ID NO: 29, wherein G at position 1 and/or S at position 2 of SEQ ID NO: 29 are optionally missing. In one embodiment, each of said ankyrin repeat domains independently comprises an amino acid sequence with at least 95% amino acid sequence identity with SEQ ID NO: 29, wherein G at position 1 and/or S at position 2 of SEQ ID NO: 29 are optionally missing. In one embodiment, each of said ankyrin repeat domains independently comprises an amino acid sequence with at least 98% amino acid sequence identity with SEQ ID NO: 29, wherein G at position 1 and/or S at position 2 of SEQ ID NO: 29 are optionally missing. Thus, in one preferred embodiment, the recombinant binding protein of the invention comprises two ankyrin repeat domains having binding specificity for CD40, wherein each of said ankyrin repeat domains independently comprises or consists of the amino acid sequence of SEQ ID NO: 29, wherein G at position 1 and/or S at position 2 of SEQ ID NO: 29 are optionally missing.

In one embodiment, said two or three or more ankyrin repeat domains are linked with a peptide linker. In one embodiment, said peptide linker is a proline-threonine rich peptide linker. In one embodiment, said peptide linker is the proline-threonine rich peptide linker of SEQ ID NO: 1 or 2. In one embodiment, said two or three or more ankyrin repeat domains are linked with the proline-threonine rich peptide linker of SEQ ID NO: 1 or 2.

In one embodiment, the recombinant binding protein of the invention further comprises a localizer molecule. In one embodiment, said localizer molecule is linked, conjugated, fused or otherwise physically attached to said CD40-specific ankyrin repeat domain or said two or three or more CD40-specific ankyrin repeat domains. In one embodiment, said localizer molecule is covalently linked to said CD40-specific ankyrin repeat domain or said two or three or more CD40-specific ankyrin repeat domains. In one embodiment, said localizer molecule is covalently linked to said CD40-specific ankyrin repeat domain or said two or three or more CD40-specific ankyrin repeat domains with a peptide linker. In one embodiment, said peptide linker is a proline-threonine rich peptide linker. In one embodiment, said peptide linker is the proline-threonine rich peptide linker of SEQ ID NO: 1 or 2. In one embodiment, said localizer molecule is covalently linked to said CD40-specific ankyrin repeat domain or said two or three or more CD40-specific ankyrin repeat domains with the proline-threonine rich peptide linker of SEQ ID NO: 1 or 2.

As shown in Example 3, one, two or three CD40-specific ankyrin repeat domains of the invention can be linked to a localizer that is capable of facilitating localized activation of CD40 by the recombinant binding protein via, for example, localizer-mediated clustering. Such localized activation, e.g. targeted to tumor tissue, might be highly beneficial for avoiding or reducing systemic activation of CD40 and resulting liver toxicity. Such embodiment allows to localize or deliver the activation of CD40 to a specific tissue by way of said localizer molecule, which binds specifically, for example, to the extracellular domain of a molecule expressed selectively in cells of the target tissue and thereby clusters the CD40-specific ankyrin repeat domain or domains to achieve CD40 activation in nearby CD40-expressing cells. For further example, numerous transmembrane proteins are known to be specifically expressed or over-expressed in tumor tissue in various types of cancers. Such tumor-specific proteins include, without limitation, anchoring proteins, receptors, enzymes, and transporter proteins such as NDC1 (TMEM48), TMEM45A, TMEM97, anoctamin-1 (TMEM16A), TMEM140, TMEM45B, αvβ3 integrin, Bombesin R, CAIX, CEA, CD13, CD44 v6, CXCR4, EGFR, ErbB-2, HER2, Emmprin, Endoglin, EpCAM, EphA2, fibronectin extra domain B (ED-B), FAP-α, Folate R, GRP78, IGF-1R, Matriptase, mesothelin, cMET/HGFR, MT1-MMP, MT6-MMP, Muc-1, PSCA, PSMA, Tn antigen, uPAR (Schmit K and Michiels C, Front. Pharmacol., 2018, 9:1345; Boonstra C M et al, Biomarkers in Cancer 2016, 8:119-133).

In one embodiment, said localizer molecule is a protein with binding specificity for a protein expressed in tumor tissue. In one embodiment, said localizer molecule is a protein with binding specificity for a tumor-specific protein. In one embodiment, said localizer molecule is a protein with binding specificity for a cell surface protein expressed in tumor tissue.

In one embodiment, said localizer molecule is an ankyrin repeat domain with binding specificity for a protein expressed in tumor tissue. In one embodiment, said localizer molecule is an ankyrin repeat domain with binding specificity for a tumor-specific protein. In one embodiment, said localizer molecule is an ankyrin repeat domain with binding specificity for a cell surface protein expressed in tumor tissue. In one most preferred embodiment, said localizer molecule is an ankyrin repeat domain with binding specificity for fibroblast activation protein (FAP). In one embodiment, said ankyrin repeat domain with binding specificity for FAP comprises the amino acid sequence of SEQ ID NO: 98.

In one embodiment, the recombinant binding protein of the invention comprises an ankyrin repeat domain having binding specificity for CD40 and further comprises a localizer molecule. In one preferred embodiment, said CD40-specific ankyrin repeat domain is an ankyrin repeat domain with binding specificity for CD40 as described more specifically in any of the aspects and embodiments herein. In one more preferred embodiment, said localizer molecule is a protein with binding specificity for a cell surface protein expressed in tumor tissue. In another more preferred embodiment, said protein with binding specificity for a cell surface protein expressed in tumor tissue is an ankyrin repeat domain with binding specificity for a cell surface protein expressed in tumor tissue. In one most preferred embodiment, said localizer molecule is an ankyrin repeat domain with binding specificity for FAP. In one embodiment, said ankyrin repeat domain having binding specificity for CD40 and said localizer ankyrin repeat domain are linked with a proline-threonine rich peptide linker. In one embodiment, said ankyrin repeat domain having binding specificity for CD40 and said localizer ankyrin repeat domain are linked with the proline-threonine rich peptide linker of SEQ ID NO: 1 or 2. In one embodiment, said localizer ankyrin repeat domain is located N-terminally of said ankyrin repeat domain having binding specificity for CD40 within said recombinant binding protein.

In one embodiment, the recombinant binding protein of the invention comprises a polypeptide consisting of two ankyrin repeat domains with binding specificity for CD40 linked with a peptide linker, and further comprises a localizer molecule. In one preferred embodiment, each of said two ankyrin repeat domains having binding specificity for CD40 is an ankyrin repeat domain with binding specificity for CD40 as described more specifically in any of the aspects and embodiments herein, which might be identical or different from each other. In one preferred embodiment, two ankyrin repeat domains with binding specificity for CD40 are linked with a proline-threonine rich peptide linker. In one embodiment, said two ankyrin repeat domains with binding specificity for CD40 are linked with the proline-threonine rich peptide linker of SEQ ID NO: 1 or 2. In a further more preferred embodiment, said localizer molecule is a protein with binding specificity for a cell surface protein expressed in tumor tissue. In another more preferred embodiment, said protein with binding specificity for a cell surface protein expressed in tumor tissue is an ankyrin repeat domain with binding specificity for a cell surface protein expressed in tumor tissue. In one most preferred embodiment, said localizer molecule is an ankyrin repeat domain with binding specificity for FAP. In one embodiment, said polypeptide and said localizer ankyrin repeat domain are linked with a proline-threonine rich peptide linker. In one embodiment, said polypeptide and said localizer ankyrin repeat domain are linked with the proline-threonine rich peptide linker of SEQ ID NO: 1 or 2. In one preferred embodiment, said localizer ankyrin repeat domain is located N-terminally of said polypeptide within said recombinant binding protein.

In one embodiment, the CD40-specific recombinant binding protein of the invention further comprises an ankyrin repeat domain with binding specificity for serum albumin. In one embodiment, the CD40-specific recombinant binding protein of the invention further comprises two ankyrin repeat domains with binding specificity for serum albumin. In one embodiment, said CD40-specific recombinant binding protein of the invention further comprises two ankyrin repeat domains with binding specificity for serum albumin, wherein one of said two ankyrin repeat domains with binding specificity for serum albumin is located N-terminally of the CD40-specific ankyrin repeat domain and wherein the other one of said two ankyrin repeat domains with binding specificity for serum albumin is located C-terminally of the CD40-specific ankyrin repeat domain. In one embodiment, the recombinant binding protein of the invention comprises two ankyrin repeat domains with binding specificity for CD40 and further comprises two ankyrin repeat domains with binding specificity for serum albumin, wherein one of said two ankyrin repeat domains with binding specificity for serum albumin is located N-terminally of said CD40-specific ankyrin repeat domains and wherein the other one of said two ankyrin repeat domains with binding specificity for serum albumin is located C-terminally of said two CD40-specific ankyrin repeat domains. Ankyrin repeat domains with binding specificity for serum albumin are able to increase the in vivo half-life of the recombinant protein of the invention.

In one embodiment, the CD40-specific recombinant binding protein of the invention further comprises a polypeptide tag. A polypeptide tag is an amino acid sequence attached to a polypeptide/protein, wherein said amino acid sequence is useful for the purification, detection, or targeting of said polypeptide/protein, or wherein said amino acid sequence improves the physicochemical behavior of the polypeptide/protein, or wherein said amino acid sequence possesses an effector function. The individual polypeptide tags of a binding protein may be connected to other parts of the binding protein directly or via peptide linkers. Polypeptide tags are all well known in the art and are fully available to the person skilled in the art. Examples of polypeptide tags are small polypeptide sequences, for example, His, HA, myc, FLAG, or Strep-tags, or polypeptides such as enzymes (for example alkaline phosphatase), which allow the detection of said polypeptide/protein, or polypeptides which can be used for targeting (such as immunoglobulins or fragments thereof) and/or as effector molecules. A particular example of His-tag that can be used in the context of the present invention is depicted in SEQ ID NO: 4.

In one embodiment, the CD40-specific recombinant binding protein of the invention further comprises a peptide linker. A peptide linker is an amino acid sequence, which is able to link, for example, two protein domains, a polypeptide tag and a protein domain, a protein domain and a non-proteinaceous compound or polymer such as polyethylene glycol, a protein domain and a biologically active molecule, a protein domain and a localizer, or two sequence tags. Peptide linkers are known to the person skilled in the art. A list of examples is provided in the description of patent application WO2002/020565. Particular examples of such linkers are glycine-serine-linkers and proline-threonine-linkers of variable lengths. Examples of a glycine-serine-linker are the amino acid sequence GS and the amino acid sequence of SEQ ID NO: 3, and examples of a proline-threonine-linker are the amino acid sequences of SEQ ID NO: 1 and SEQ ID NO: 2.

In another aspect, the invention provides a nucleic acid encoding the amino acid sequence of the ankyrin repeat domain or the recombinant binding protein of the present invention. In one embodiment, the invention provides a nucleic acid encoding the amino acid sequence of a recombinant binding protein of the present invention. In one embodiment, the invention provides a nucleic acid encoding an ankyrin repeat domain with binding specificity for CD40 of the present invention. In one embodiment, said nucleic acid encodes the amino acid sequence of the ankyrin repeat domain of any one of SEQ ID NOs: 16 to 35. In one preferred embodiment, said nucleic acid encodes the amino acid sequence of the ankyrin repeat domain of any one of SEQ ID NOs: 22, 29 and 31. In one embodiment, said nucleic acid encodes the amino acid sequence of the ankyrin repeat domain of SEQ ID NO: 22. In one embodiment, said nucleic acid encodes the amino acid sequence of the ankyrin repeat domain of SEQ ID NO: 29. In one embodiment, said nucleic acid encodes the amino acid sequence of the ankyrin repeat domain of SEQ ID NO: 31. In one more preferred embodiment said nucleic acid is selected from the group consisting of SEQ ID NOs: 36, 37 and 38. In one embodiment, said nucleic acid comprises or consists of the nucleic acid sequence of SEQ ID NO: 36, which encodes the amino acid sequence of SEQ ID NO: 29. In one embodiment, said nucleic acid comprises or consists of the nucleic acid sequence of SEQ ID NO: 37, which encodes the amino acid sequence of SEQ ID NO: 22. In one embodiment, said nucleic acid comprises or consists of the nucleic acid sequence of SEQ ID NO: 38, which encodes the amino acid sequence of SEQ ID NO: 31.

Furthermore, the invention provides vectors comprising any nucleic acid of the invention. Nucleic acids are well known to the skilled person in the art. In the examples, nucleic acids were used to produce designed ankyrin repeat domains or recombinant binding proteins of the invention in E. coli.

In one aspect, the invention provides a pharmaceutical composition comprising the recombinant binding protein and/or the ankyrin repeat domain of the present invention, and/or the nucleic acid encoding a recombinant binding protein and/or a designed ankyrin repeat domain of the present invention, and optionally a pharmaceutically acceptable carrier and/or diluent.

In one embodiment, the invention provides a pharmaceutical composition comprising the recombinant binding protein or the nucleic acid encoding a recombinant binding protein of the present invention, and optionally a pharmaceutically acceptable carrier and/or diluent.

Pharmaceutically acceptable carriers and/or diluents are known to the person skilled in the art and are explained in more detail below. Even further, a diagnostic composition is provided comprising one or more of the above mentioned recombinant binding proteins and/or designed ankyrin repeat domains, and/or nucleic acids, in particular recombinant binding proteins and/or nucleic acids of the present invention.

A pharmaceutical composition comprises a recombinant binding protein, and/or an ankyrin repeat domain, and/or a nucleic acid, preferably a recombinant binding protein and/or a nucleic acid, as described herein and a pharmaceutically acceptable carrier, excipient or stabilizer, for example as described in Remington's Pharmaceutical Sciences 16^{th} edition, Osol, A. Ed., 1980.

Suitable carriers, excipients or stabilizers known to one of skill in the art include, for example, saline, Ringer's solution, dextrose solution, Hank's solution, fixed oils, ethyl oleate, 5% dextrose in saline, substances that enhance isotonicity and chemical stability, buffers and preservatives. Other suitable carriers include any carrier that does not itself induce the production of antibodies harmful to the individual receiving the composition such as proteins, polysaccharides, polylactic acids, polyglycolic acids, polymeric amino acids and amino acid copolymers. A pharmaceutical composition may also be a combination formulation, comprising an additional active agent, such as an anti-cancer agent or an anti-angiogenic agent, or an additional bioactive compound.

The formulations to be used for in vivo administration must be aseptic or sterile. This is readily accomplished by filtration through sterile filtration membranes.

One embodiment of the present invention provides the use of a recombinant binding protein of the present invention comprising an ankyrin repeat domain having binding specificity for CD40 and further comprising an ankyrin repeat domain with binding specificity for serum albumin for manufacturing a pharmaceutical composition, wherein said recombinant binding protein exhibits an increased terminal half-life, preferably an increased terminal half-life of at least 5%, preferably 10%, 15%, 20%, 25%, 30%, 35%, 40%, 45%, 50%, 60%, 70%, 80%, 90%, 100%, 150%, 200%, or 250%, compared to a corresponding recombinant binding protein comprising said ankyrin repeat domain with binding specificity for CD40 but not said ankyrin repeat domain with binding specificity for serum albumin. In one embodiment of the invention, a recombinant binding protein comprises an ankyrin repeat domain having binding specificity for CD40 and further comprises two ankyrin repeat domains with binding specificity for serum albumin.

In one embodiment, a pharmaceutical composition comprises at least one recombinant binding protein as described herein and a detergent such as nonionic detergent, a buffer such as phosphate buffer, and a sugar such as sucrose. In one embodiment, such a composition comprises recombinant binding proteins as described above and PBS.

In another aspect, the invention provides a method of localized activation of CD40 in CD40-expressing cells in a mammal, including a human, the method comprising the step of administering to said mammal the recombinant binding protein of the invention. In one preferred embodiment, said recombinant binding protein comprises an ankyrin repeat domain with binding specificity for CD40 and further comprises a localizer molecule. In one embodiment, said localizer molecule is a binding protein having binding specificity for a target different from CD40. In one embodiment, said mammal is a human. In one embodiment, said CD40-expressing cells are located in a tumor, including in a primary tumor, metastasis and/or tumor stroma.

In another aspect, the invention provides a method of treating a medical condition, the method comprising the step of administering to a patient in need thereof a therapeutically effective amount of the recombinant binding protein of the invention, the nucleic acid of the invention or the pharmaceutical composition of the invention. In a preferred embodiment, said recombinant binding protein comprises a localizer molecule. In one preferred embodiment, said medical condition is a cancer.

In another aspect, the invention provides a method of treating a medical condition, the method comprising the step of administering to a patient in need thereof a therapeutically effective amount of the recombinant binding protein of the invention comprising a localizer molecule, wherein said localizer molecule is effective in localizing said binding protein to target tissue, and wherein said localization of said binding protein results in activation of CD40 in CD40-expressing cells in the target tissue. In one embodiment, said localizer molecule is a binding protein having binding specificity for a cell surface protein expressed in a tumor, wherein said cell surface protein is different from CD40. In one embodiment, said CD40-expressing cells are located in a tumor, including in a primary tumor, metastasis and/or tumor stroma. These embodiments thus allow to take advantage of the localizer's restricted expression in a tumor by localizing the activation of CD40 by the recombinant binding protein of the invention to the tumor.

In another aspect, the invention provides a method of diagnosing a medical condition in a patient, the method comprising the step of administering the recombinant binding protein of the invention comprising an ankyrin repeat domain with binding specificity for human CD40 to a patient in need of said diagnosis or to a body fluid or tissue sample of a patient in need of said diagnosis. In one embodiment, said body fluid is blood plasma or a derivative thereof. In one embodiment, said body fluid is blood serum. In one embodiment, said tissue is tumor tissue. In one embodiment, said medical condition is cancer. In another embodiment, said medical condition is an autoimmune disease.

In one embodiment, the invention provides the use of a pharmaceutical composition, or a recombinant binding protein according to the present invention for the treatment of a disease. For that purpose, the pharmaceutical composition, or the recombinant binding protein according to the present invention is administered, to a patient in need thereof, in a therapeutically effective amount. Administration may include topical administration, oral administration, and parenteral administration. The typical route of administration is parenteral administration. In parental administration, the pharmaceutical composition of this invention will be formulated in a unit dosage injectable form such as a solution, suspension or emulsion, in association with the pharmaceutically acceptable excipients as defined above. The dosage and mode of administration will depend on the individual to be treated and the particular disease.

Further, any of the above mentioned pharmaceutical composition or recombinant binding protein is considered for the treatment of a disorder.

In one embodiment, said recombinant binding protein or such other pharmaceutical composition described herein is applied intravenously. For parenteral application, the recombinant binding protein or said pharmaceutical composition can be injected as bolus injection or by slow infusion at a therapeutically effective amount.

In one embodiment, the invention provides a method of treatment of a medical condition, the method comprising the step of administering, to a patient in need of such a treatment, a therapeutically effective amount of a recombinant binding protein of the invention. In one embodiment, the invention provides a method of treatment of a medical condition, the method comprising the step of administering, to a patient in need of such a treatment, a therapeutically effective amount of a pharmaceutical composition of the invention. In one embodiment, the invention provides the use of a pharmaceutical composition of the present invention for the treatment of a disease. In one embodiment, the invention provides a pharmaceutical composition for use in the treatment of a disease. In one embodiment, the invention provides a pharmaceutical composition for use in the treatment of a medical condition. In one embodiment, the invention provides a nucleic acid for use in the treatment of a disease. In one embodiment, the invention provides the use of said pharmaceutical composition, recombinant binding protein, or nucleic acid molecule, as medicament for the treatment of a disease. In one embodiment, the invention provides the use of said pharmaceutical composition, recombinant binding protein, or nucleic acid molecule, for manufacturing of a medicament. In one embodiment, the invention provides the use of said pharmaceutical composition, recombinant binding protein, or nucleic acid molecule, for manufacturing of a medicament for the treatment of a disease. In one embodiment, the invention r provides a process for the manufacturing of a medicament for the treatment of a disease, wherein said pharmaceutical composition, recombinant binding protein, or nucleic acid molecule is active ingredient of the medicament. In one embodiment, the invention provides a process of treatment of a disease using said pharmaceutical composition, recombinant binding protein, or nucleic acid molecule.

In particular, the invention provides the treatment of a medical condition using a pharmaceutical composition of the present invention, wherein said medical condition is cancer.

The use of the recombinant binding protein of the present invention or said pharmaceutical compositions for the treatment of cancer diseases can also be in combination with one or more other therapies known in the art. The term “use in combination with”, as used herein, shall refer to a co-administration, which is carried out under a given regimen. This includes synchronous administration of the different compounds as well as time-shifted administration of the different compounds (e.g. compound A is given once and compound B is given several times thereafter, or vice versa, or both compounds are given synchronously and one of the two is also given at later stages).

In a further embodiment, the invention provides the use of a recombinant binding protein of the invention for the manufacture of a medicament that is used for the treatment of a medical condition, preferably a neoplastic disease, more preferably cancer.

In one embodiment, the invention provides the use of a pharmaceutical composition of the invention for the manufacture of a medicament that is used for the treatment of a medical condition, which may be a neoplastic disease, in particular cancer.

In one embodiment the invention provides a recombinant binding protein comprising any of the above mentioned ankyrin repeat domains.

In one embodiment, the invention provides a kit comprising said recombinant binding protein. In one embodiment, the invention provides a kit comprising a nucleic acid encoding said recombinant binding protein. In one embodiment, the invention provides a kit comprising said pharmaceutical composition. In one embodiment, the invention provides a kit comprising said recombinant binding protein, and/or a nucleic acid encoding said recombinant binding protein, and/or said pharmaceutical composition. In one embodiment, the invention provides a kit comprising the recombinant binding protein comprising a CD40-specific ankyrin repeat domain, for example the CD40-specific ankyrin repeat domain of SEQ ID NO: 29, and/or a nucleic acid encoding the recombinant binding protein comprising a CD40-specific ankyrin repeat domain, for example the nucleic acid of SEQ ID NO: 36, and/or a pharmaceutical composition comprising said recombinant binding protein comprising a CD40-specific ankyrin repeat domain, and/or a nucleic acid encoding said recombinant binding protein comprising a CD40-specific ankyrin repeat domain.

In one embodiment, the invention provides a method for producing the recombinant binding protein of the present invention. In one embodiment, the invention provides a method for producing a recombinant binding protein, for example a recombinant binding protein comprising the amino acid sequence of SEQ ID NO: 29, the method comprising the steps of (i) expressing said recombinant binding protein in bacteria, and (ii) purifying said recombinant binding protein using chromatography. Said method may comprise additional steps.

The invention is not restricted to the particular embodiments described in the Examples.

This specification refers to a number of amino acid sequences, nucleic acid sequences and SEQ ID NOs that are disclosed in the appended Sequence Listing, which is herewith incorporated by reference in its entirety.

### Definitions

Unless defined otherwise herein, all technical and scientific terms used herein shall have the meanings that are commonly understood by those of ordinary skill in the art to which the present invention belongs.

In the context of the present invention the term “protein” refers to a molecule comprising a polypeptide, wherein at least part of the polypeptide has, or is able to acquire, a defined three-dimensional arrangement by forming secondary, tertiary, and/or quaternary structures within a single polypeptide chain and/or between multiple polypeptide chains. If a protein comprises two or more polypeptide chains, the individual polypeptide chains may be linked non-covalently or covalently, e.g. by a disulfide bond between two polypeptides. A part of a protein, which individually has, or is able to acquire, a defined three-dimensional arrangement by forming secondary and/or tertiary structure, is termed “protein domain”. Such protein domains are well known to the practitioner skilled in the art.

The term “recombinant” as used in recombinant protein, recombinant polypeptide and the like, means that said protein or polypeptide is produced by the use of recombinant DNA technologies well known to the practitioner skilled in the art. For example, a recombinant DNA molecule (e.g. produced by gene synthesis) encoding a polypeptide can be cloned into a bacterial expression plasmid (e.g. pQE30, QIAgen), yeast expression plasmid, mammalian expression plasmid, or plant expression plasmid, or a DNA enabling in vitro expression. If, for example, such a recombinant bacterial expression plasmid is inserted into appropriate bacteria (e.g. Escherichia coli), these bacteria can produce the polypeptide(s) encoded by this recombinant DNA. The correspondingly produced polypeptide or protein is called a recombinant polypeptide or recombinant protein.

In the context of the present invention, the term “binding protein” refers to a protein comprising a binding domain. A binding protein may also comprise two, three, four, five or more binding domains. Preferably, said binding protein is a recombinant binding protein. Binding proteins of the instant invention comprise at least one ankyrin repeat domain with binding specificity for CD40.

Furthermore, any such binding protein may comprise additional polypeptides (such as e.g. polypeptide tags, peptide linkers, fusion to other proteinaceous domains with binding specificity, cytokines, hormones, or antagonists), or chemical modifications (such as coupling to polyethylene-glycol, toxins (e.g. DM1 from Immunogen), small molecules, antibiotics and alike) well known to the person skilled in the art. A binding protein of the instant invention may comprise a localizer molecule.

The term “binding domain” means a protein domain exhibiting binding specificity for a target. Preferably, said binding domain is a recombinant binding domain. More preferably, said binding domain is an ankyrin repeat domain with binding specificity for a specific target. Examples of specific targets to which an ankyrin binding domain of the invention may bind to include but are not limited to CD40, FAP and serum albumin.

The term “target” refers to an individual molecule such as a nucleic acid molecule, a polypeptide or protein, a carbohydrate, or any other naturally occurring molecule, including any part of such individual molecule, or to complexes of two or more of such molecules, or to a whole cell or a tissue sample, or to any non-natural compound. Preferably, a target is a naturally occurring or non-natural polypeptide or protein, or a polypeptide or protein containing chemical modifications, for example, naturally occurring or non-natural phosphorylation, acetylation, or methylation. In the context of the present invention, CD40 and CD40-expressing cells are targets of CD40-specific binding proteins and localizer target proteins and cells are targets of localizers.

The terms “nucleic acid” or “nucleic acid molecule” refer to a polynucleotide molecule, which may be a ribonucleic acid (RNA) or deoxyribonucleic acid (DNA) molecule, either single stranded or double stranded, and includes modified and artificial forms of DNA or RNA. A nucleic acid molecule may either be present in isolated form, or be comprised in recombinant nucleic acid molecules or vectors.

In the context of the present invention, the term “polypeptide” relates to a molecule consisting of a chain of multiple, i.e. two or more, amino acids linked via peptide bonds. Preferably, a polypeptide consists of more than eight amino acids linked via peptide bonds. The term “polypeptide” also includes multiple chains of amino acids, linked together by S-S bridges of cysteines. Polypeptides are well-known to the person skilled in the art.

Patent application WO2002/020565 and Forrer et al., 2003 (Forrer, P., Stumpp, M. T., Binz, H. K., Plückthun, A., 2003. FEBS Letters 539, 2-6), contain a general description of repeat protein features and repeat domain features, techniques and applications. The term “repeat protein” refers to a protein comprising one or more repeat domains. Preferably, a repeat protein comprises one, two, three, four, five or six repeat domains. Furthermore, said repeat protein may comprise additional non-repeat protein domains, polypeptide tags and/or peptide linkers. The repeat domains can be binding domains.

The term “repeat domain” refers to a protein domain comprising two or more consecutive repeat modules as structural units, wherein said repeat modules have structural and sequence homology. Preferably, a repeat domain further comprises an N-terminal and/or a C-terminal capping module. For clarity, a capping module can be a repeat module. Such repeat domains, repeat modules, and capping modules, sequence motives, as well as structural homology and sequence homology are well known to the practitioner in the art from examples of ankyrin repeat domains (WO2002/020565), leucine-rich repeat domains (WO2002/020565), tetratricopeptide repeat domains (Main, E. R., Xiong, Y., Cocco, M. J., D'Andrea, L., Regan, L., Structure 11 (5), 497-508, 2003), and armadillo repeat domains (WO2009/040338). It is further well known to the practitioner in the art, that such repeat domains are different from proteins comprising repeated amino acid sequences, where every repeated amino acid sequence is able to form an individual domain (for example FN3 domains of Fibronectin).

The term “designed” as used in designed repeat protein, designed repeat domain and the like refers to the property that such repeat proteins and repeat domains, respectively, are man-made and do not occur in nature. The binding proteins of the instant invention are designed repeat proteins and they comprise at least one designed ankyrin repeat domain.

The term “target interaction residues” refers to amino acid residues of a repeat module, which contribute to the direct interaction with a target.

The term “framework residues” refers to amino acid residues of a repeat module, which contribute to the folding topology, i.e. which contribute to the fold of said repeat module or which contribute to the interaction with a neighboring module. Such contribution may be the interaction with other residues in the repeat module, or the influence on the polypeptide backbone conformation as found in a-helices or B-sheets, or the participation in amino acid stretches forming linear polypeptides or loops.

Such framework and target interaction residues may be identified by analysis of the structural data obtained by physicochemical methods, such as X-ray crystallography, NMR and/or CD spectroscopy, or by comparison with known and related structural information well known to practitioners in structural biology and/or bioinformatics.

The term “repeat modules” refers to the repeated amino acid sequence and structural units of the designed repeat domains, which are originally derived from the repeat units of naturally occurring repeat proteins. Each repeat module comprised in a repeat domain is derived from one or more repeat units of a family or subfamily of naturally occurring repeat proteins, e.g. the family of ankyrin repeat proteins. Furthermore, each repeat module comprised in a repeat domain may comprise a “repeat sequence motif” deduced from homologous repeat modules obtained from repeat domains selected on a target and having the same target specificity.

Accordingly, the term “ankyrin repeat module” refers to a repeat module, which is originally derived from the repeat units of naturally occurring ankyrin repeat proteins. Ankyrin repeat proteins are well known to the person skilled in the art.

Repeat modules may comprise positions with amino acid residues which have not been randomized in a library for the purpose of selecting target-specific repeat domains (“non-randomized positions”) and positions with amino acid residues which have been randomized in the library for the purpose of selecting target-specific repeat domains (“randomized positions”). The non-randomized positions comprise framework residues. The randomized positions comprise target interaction residues. “Have been randomized” means that two or more amino acids were allowed at an amino acid position of a repeat module, for example, wherein any of the usual twenty naturally occurring amino acids were allowed, or wherein most of the twenty naturally occurring amino acids were allowed, such as amino acids other than cysteine, or amino acids other than glycine, cysteine and proline. For the purpose of this patent application, amino acid residues 3, 4, 6, 14 and 15 of SEQ ID NOs: 39 to 95 are randomized positions of the ankyrin repeat modules of the instant invention.

The term “repeat sequence motif” refers to an amino acid sequence, which is deduced from one or more repeat modules. Preferably, said repeat modules are from repeat domains having binding specificity for the same target. Such repeat sequence motifs comprise framework residue positions and target interaction residue positions. Said framework residue positions correspond to the positions of framework residues of the repeat modules. Likewise, said target interaction residue positions correspond to the positions of target interaction residues of the repeat modules. Repeat sequence motifs comprise non-randomized positions and randomized positions.

The term “repeat unit” refers to amino acid sequences comprising sequence motifs of one or more naturally occurring proteins, wherein said “repeat units” are found in multiple copies, and exhibit a defined folding topology common to all said motifs determining the fold of the protein. Examples of such repeat units include leucine-rich repeat units, ankyrin repeat units, armadillo repeat units, tetratricopeptide repeat units, HEAT repeat units, and leucine-rich variant repeat units.

The term “has binding specificity for a target”, “specifically binding to a target”, “binding to a target with high specificity”, “specific for a target” or “target specificity” and the like means that a binding protein or binding domain binds in PBS to a target with a lower dissociation constant (i.e. it binds with higher affinity) than it binds to an unrelated protein such as the E. coli maltose binding protein (MBP). Preferably, the dissociation constant (“K_{D}”) in PBS for the target is at least 10²; more preferably, at least 10³; more preferably, at least 10⁴; or more preferably, at least 10⁵ times lower than the corresponding dissociation constant for MBP. Methods to determine dissociation constants of protein-protein interactions, such as surface plasmon resonance (SPR) based technologies (e.g. SPR equilibrium analysis) or isothermal titration calorimetry (ITC) are well known to the person skilled in the art. The measured K_{D} values of a particular protein-protein interaction can vary if measured under different conditions (e.g., salt concentration, pH). Thus, measurements of K_{D} values are preferably made with standardized solutions of protein and a standardized buffer, such as PBS. A typical and preferred determination of dissociation constants (K_{D}) of the recombinant binding proteins of the invention with binding specificity for CD40 is in PBS and by Surface Plasmon Resonance (SPR).

The term “polypeptide tag” refers to an amino acid sequence attached to a polypeptide/protein, wherein said amino acid sequence is useful for the purification, detection, or targeting of said polypeptide/protein, or wherein said amino acid sequence improves the physicochemical behavior of the polypeptide/protein, or wherein said amino acid sequence possesses an effector function. The individual polypeptide tags, moieties and/or domains of a binding protein may be connected to each other directly or via polypeptide linkers. These polypeptide tags are all well known in the art and are fully available to the person skilled in the art. Examples of polypeptide tags are small polypeptide sequences, for example, His (e.g. the His-tag consisting of SEQ ID NO: 4), myc, FLAG, or Strep-tags or moieties such as enzymes (for example enzymes like alkaline phosphatase), which allow the detection of said polypeptide/protein, or moieties which can be used for targeting (such as immunoglobulins or fragments thereof) and/or as effector molecules.

The term “polypeptide linker” refers to an amino acid sequence, which is able to link, for example, two protein domains, a polypeptide tag and a protein domain, a protein domain and a non-polypeptide moiety such as polyethylene glycol or two polypeptide tags tags. Such additional domains, tags, non-polypeptide moieties and linkers are known to the person skilled in the relevant art. Examples of such polypeptide linkers are the linkers consisting of SEQ ID NOs: 1 and 2.

The term “about” means the mentioned value+/−20%; for example “about 50” shall mean 40 to 60.

The term “PBS” means a phosphate buffered water solution containing 137 mM NaCl, 10 mM phosphate and 2.7 mM KCl and having a pH of 7.4.

The term “serum albumin” as used herein includes but is not limited to mouse serum albumin, cynomolgus monkey serum albumin and human serum albumin. The term “mouse serum albumin” refers to UniProt accession number P07724, the term “cynomolgus monkey serum albumin” (i.e. Macaca fascicularis) refers to UniProt accession number A2V9Z4, and the term “human serum albumin” refers to UniProt accession number P02768.

Preferably, clearance, and/or exposure, and/or terminal half-life are assessed in a mammal, more preferably mouse and/or cynomolgus monkey, more preferably cynomolgus monkey. Preferably, when measuring the clearance, and/or exposure, and/or terminal half-life in mouse, the evaluation is done considering the data up to 48 h post-injection. More preferably, the evaluation of terminal half-life in mouse is calculated from 24 h to 48 h. Preferably, when measuring the clearance, and/or exposure, and/or terminal half-life in cynomolgus monkey, the evaluation is done considering the data up to day 7 post-injection. More preferably, the evaluation of terminal half-life in cynomolgus monkey is calculated from day 1 to day 5. The person skilled in the art further is able to identify effects such as target-mediated clearance and consider them when calculating the terminal half-life. The term “terminal half-life” of a drug such as a recombinant binding protein of the invention refers to the time required to reach half the plasma concentration of the drug applied to a mammal after reaching pseudo-equilibrium (for example calculated from 24 hours to 48 hours in mouse or calculated from day 1 to day 5 in cynomolgus monkey). Terminal half-life is not defined as the time required to eliminate half the dose of the drug administered to the mammal. The term terminal half-life is well known to the person skilled in the art. Preferably, pharmacokinetic comparison is done at any dose, more preferably at equivalent dose (i.e. same mg/kg dose) or equimolar dose (i.e. same mol/kg dose), more preferably at equimolar dose (i.e. same mol/kg dose). It is understood by the person skilled in the art that equivalent and/or equimolar dosing in animals is subject to experimental dose variations of at least 20%, more preferably 30%, 40%, 50%, 60%, 70%, 80%, 90%, or 100%. Preferably, a dose used for pharmacokinetic measurement is selected from 0.001 to 1000 mg/kg, more preferably 0.01 to 100 mg/kg, more preferably 0.1 to 50 mg/kg, more preferably 0.5 to 10 mg/kg.

The term “CD40” and “CD40 receptor” are used interchangeably in the present application, and refer to any form of CD40 receptor, as well as to variants, isoforms, and species homologs thereof that retain at least a part of the activity of CD40 receptor. Accordingly, a binding protein, as defined and disclosed herein, may also bind CD40 from species other than human. In other cases, a binding protein may be completely specific for the human CD40 and may not exhibit species or other types of cross-reactivity. Unless indicated differently, such as by specific reference to human CD40, CD40 includes all mammalian species of native sequence CD40, e.g., human, canine, feline, equine and bovine. An amino acid sequence of human CD40 is shown in NCBI Reference Sequence NP_001241.1, in Uniprot P25942 and in SEQ ID NO: 96. The costimulatory receptor CD40 is a 48-kDa type I transmembrane protein and contains a 173 amino acids extracellular domain (SEQ ID NO: 97), a 22 amino acids transmembrane domain, and a 62 amino acids intracellular domain in human. The precursor further contains a 20 amino acids leader sequence. In regard to expression pattern, CD40 was initially characterized on B cells and is also expressed on dendritic cells, monocytes, platelets, and macrophages as well as by non-hematopoietic cells such as myofibroblasts, fibroblasts, epithelial, and endothelial cells (Elgueta et al., Immunol Rev. 2009 May; 229 (1)).

“CD40 agonist” as used herein means any chemical compound or biological molecule, which upon binding to CD40, (1) stimulates or activates CD40, (2) enhances, increases, promotes, induces, or prolongs an activity, function, or presence of CD40, or (3) enhances, increases, promotes, or induces the expression of CD40. In any of the treatment methods, medicaments, pharmaceutical compositions and uses of the present invention in which a human individual is being treated, the CD40 agonists increase a CD40-mediated response. In some embodiments of the treatment methods, medicaments, pharmaceutical compositions and uses of the present invention, CD40 agonists markedly enhance the downstream signaling by CD40, resulting in anti-tumor activity in various models.

The term “localizing” or “delivering” as interchangeably used herein in the context of a “localizer molecule” or “localizer” comprised by a CD40-specific binding protein of the invention refers to increased localization of such CD40-specific binding protein comprising a localizer, as compared to when the binding protein does not comprise the localizer, to localizer target cells or tissue in a mammal. The term also refers to targeting a CD40-specific binding protein to the site of localizer target cells or tissue in a mammal, wherein the CD40-specific binding protein comprises the localizer. The term preferably further encompasses the accumulation and/or retention of a CD40-specific binding protein comprising a localizer at the site of localizer target cells or tissue in a mammal. The term also preferably encompasses the localized activation of CD40 in CD40-expressing cells induced by a CD40-specific binding protein of the invention comprising a localizer at or nearby the site of localizer target cells or tissue in a mammal. Such localized activation may occur, for example, through clustering of CD40 upon binding of the CD40-specific binding protein comprising the localizer, wherein the clustering is mediated by binding of the localizer to its target cells or tissue. As used in this paragraph, “mammal” encompasses human. The result of “localizing” may be measured by various means well known to one of skill in the art. As an example, “localizing” may be measured by determining the organ-to-blood ratio of a binding protein of the invention linked to a localizer, according to methods well known in the art. In one embodiment of the invention, the effect of a localizer on “localizing” a CD40-specific binding protein comprising a localizer is exhibited by an increased organ-to-blood ratio of at least 5%, 10%, 15%, 20%, 25%, 30%, 35%, 40%, 45%, 50%, 60%, 70%, 80%, 90%, 100%, 150%, 200%, 250%, or 300% compared to a corresponding CD40-specific binding protein that does not comprise the localizer.

The terms “localizer molecule” and “localizer” are used interchangeably herein and are intended to encompass any molecule that can be comprised by a CD40-specific binding protein of the invention and that is capable of localizing or delivering such a CD40-specific binding protein of the invention comprising it to target cells or tissue in a mammal, including, e.g., a human, wherein the localizer binds to the target cells or tissue. A localizer can be linked, conjugated, fused or otherwise physically attached to a CD40-specific ankyrin repeat domain of the invention. The terms “localizer molecule” and “localizer” encompass polynucleotides, peptides/polypeptides and/or chemical or biochemical agents that have these properties. The term “polynucleotides” generally refers to DNA or RNA, and includes modified and artificial forms of DNA or RNA. The term “peptide” refers to a peptide chain of 4 to 600 amino acids long, such as 4 to 200 amino acids long, and therefore encompasses polypeptides and proteins. The term encompasses any naturally occurring or man-made binding proteins, binding domains, growth factor receptors or fragments or ligands thereof, cytokines, polypeptide hormones, antibodies, antibody-like proteins based on scaffolds, immunomodulatory proteins, etc. Furthermore, the term “peptide” also encompasses peptides modified by, e.g, glycosylation, and proteins comprising two or more polypeptide chains, each of length of 4 to 600 amino acids long, cross-linked by, e.g., disulphide bonds, such as, e.g., insulin and immunoglobulins. The term “chemical or biochemical agent” is intended to include any naturally occurring or synthetic compound that may be administered to a recipient. In a preferred embodiment, the localizer is a target-specific ankyrin repeat domain. In one more preferred embodiment, the localizer molecule is an ankyrin repeat domain with binding specificity for FAP.

The term “FAP” as used herein refers to Fibroblast-Activation Protein. Fibroblast-activation protein a (FAP), also known as Seprase, is a type II integral membrane serine peptidase. FAP belongs to the dipeptidyl peptidase IV family (Yu et al., FEBS J. 277, 1126-1144 (2010)). It is a 170 kDa homodimer containing two N-glycosylated subunits with a large C-terminal extracellular domain, in which the enzyme's catalytic domain is located (Scanlan et al., Proc Natl Acad Sci USA 91: 5657-5661 (1994); Wonganu et al., Biochim Biophys Acta 1858 (8): 1876-82 (2016)). FAP, in its glycosylated form, has both post-prolyl dipeptidyl peptidase and gelatinase activities (Sun et al., Protein Expr Purif 24, 274-281 (2002)). Homologues of human FAP were found in several species, including mice and cynomolgus monkeys (Macaca fascicularis). FAP is expressed selectively in reactive stromal fibroblasts of more than 90% of epithelial malignancies (primary and metastatic) examined, including lung, colorectal, bladder, ovarian and breast carcinomas, and in malignant mesenchymal cells of bone and soft tissue sarcomas, while it is generally absent from normal adult tissues (Brennen et al., Mol. Cancer Ther. 11 (2): 257-266 (2012); Garin-Chesa et al., Proc Natl Acad Sci USA 87, 7235-7239 (1990); Rettig et al., Cancer Res. 53:3327-3335 (1993); Rettig et al., Proc Natl Acad Sci USA 85, 3110-3114 (1988)). FAP is also expressed on certain malignant tumor cells. Due to its expression in many common cancers and its restricted expression in normal tissues, FAP has been considered a promising antigenic target for imaging, diagnosis and therapy of a variety of cancers.

The term “CD40-expressing cells” as used herein refers to any cells expressing CD40 on the cell surface, including, but not limited, to B cells, dendritic cells, monocytes, platelets, and macrophages as well as non-hematopoietic cells such as myofibroblasts, fibroblasts, epithelial, and endothelial cells.

The term “treatment” or “treating” refers to both therapeutic treatment and prophylactic or preventative measures. Those in need of treatment include those who have already the disorder as well as those in which the disorder is to be prevented.

The terms “medical condition”, “disorder” and “disease” are used interchangeably herein and include autoimmune disorders, inflammatory disorders, retinopathies (particularly proliferative retinopathies), neurodegenerative disorders, infections, metabolic diseases, and neoplastic diseases. Any of the recombinant binding proteins described herein may be used for the preparation of a medicament for the treatment of such a disorder, particularly a disorder selected from the group comprising: an autoimmune disorder, an inflammatory disorder, an immune disorder, and a neoplastic disease. A “medical condition” may be one that is characterized by inappropriate cell proliferation. A medical condition may be a hyperproliferative condition. The invention particularly provides a method of treating a medical condition, the method comprising the step of administering, to a patient in need of such treatment, a therapeutically effective amount of a recombinant binding protein or said pharmaceutical composition of the invention. In a preferred embodiment said medical condition is a neoplastic disease. The term “neoplastic disease”, as used herein, refers to an abnormal state or condition of cells or tissue characterized by rapidly proliferating cell growth or neoplasm. In one embodiment said medical condition is a malignant neoplastic disease. In one embodiment said medical condition is a cancer. The term “therapeutically effective amount” refers to the amount sufficient to induce a desired biological, pharmacological, or therapeutic outcome in a subject. A therapeutically effective amount in the context of the invention means a sufficient amount of the binding protein to treat or prevent a disease or disorder at a reasonable benefit/risk ratio applicable to any medical treatment.

The terms “cancer” and “cancerous” are used herein to refer to or describe the physiological condition in mammals that is typically characterized by unregulated cell growth. Cancer encompasses solid tumors and liquid tumors, as well as primary tumors and metastases. A “tumor” comprises one or more cancerous cells. Solid tumors typically also comprise tumor stroma. Examples of cancer include, but are not limited to, primary and metastatic carcinoma, lymphoma, blastoma, sarcoma, and leukemia, and any other epithelial and lymphoid malignancies. More particular examples of such cancers include brain cancer, bladder cancer, breast cancer, ovarian cancer, clear cell kidney cancer, head/neck squamous cell carcinoma, lung adenocarcinoma, lung squamous cell carcinoma, malignant melanoma, non-small-cell lung cancer (NSCLC), ovarian cancer, pancreatic cancer, prostate cancer, renal cell carcinoma, small-cell lung cancer (SCLC), triple negative breast cancer, acute lymphoblastic leukemia (ALL), acute myeloid leukemia (AML), chronic lymphocytic leukemia (CLL), chronic myeloid leukemia (CML), diffuse large B-cell lymphoma (DLBCL), follicular lymphoma, Hodgkin's lymphoma (HL), mantle cell lymphoma (MCL), multiple myeloma (MM), myelodysplastic syndrome (MDS), non-Hodgkin's lymphoma (NHL), Squamous Cell Carcinoma of the Head and Neck (SCCHN), chronic myelogenous leukemia (CML), small lymphocytic lymphoma (SLL), malignant mesothelioma, colorectal cancer, or gastric cancer.

The term “improved pharmacokinetic properties” as used herein refers to an increased area under the curve, a reduced clearance, or an increased terminal half-life. These parameters of pharmacokinetic properties and ways to determine them are well known in the art (see, e.g., Mahmood, I., Methods to determine pharmacokinetic profiles of therapeutic proteins, Drug Discov Today: Technol (2009), doi:10.1016/j.ddtec.2008.12.001).

In the context of the present invention, the letter “X” in an amino acid sequence, such as, e.g., SEQ ID NO: 8, represents any amino acids. In the context of the present invention, the term “any amino acids” preferably means any of the 20 most often naturally occurring amino acids, namely alanine (ala; A), arginine (arg; R), asparagine (asn, N), aspartic acid (asp, D), cysteine (cys, C), glutamine (gln, Q), glutamic acid (glu, E), glycine (gly, G), histidine (his, H), isoleucine (ile, I), leucine (leu, L), lysine (lys, K), methionine (met, M), phenylalanine (phe, F), proline (pro, P), serine (ser, S), threonine (thr, T), tryptophan (trp, W), tyrosine (tyr, Y), valine (val, V).

The term “mammal” for purposes of treatment refers to any animal classified as a mammal, including human, domestic and farm animals, nonhuman primates, and zoo, sports, or pet animals, such as dogs, horses, cats, cows, etc.

### EXAMPLES

Starting materials and reagents disclosed below are known to those skilled in the art, are commercially available and/or can be prepared using well-known techniques.

### Materials

Chemicals were purchased from Sigma-Aldrich (USA). Oligonucleotides were from Microsynth (Switzerland). Unless stated otherwise, DNA polymerases, restriction enzymes and buffers were from New England Biolabs (USA) or Fermentas/Thermo Fisher Scientific (USA). Inducible E. coli expression strains were used for cloning and protein production, e.g. E. coli XL1-blue (Stratagene, USA) or BL21 (Novagen, USA). Recombinant Fc fusion protein of the extracellular domain of human CD40 was purchased from ACRO Biosystems.

### Molecular Biology

Unless stated otherwise, methods are performed according to known protocols (see, e.g., Sambrook J., Fritsch E. F. and Maniatis T., Molecular Cloning: A Laboratory Manual, Cold Spring Harbor Laboratory 1989, New York).

### Designed Ankyrin Repeat Protein Libraries

Methods to generate designed ankyrin repeat protein libraries have been described, e.g. in U.S. Pat. No. 7,417,130; Binz et al., J. Mol. Biol. 332, 489-503 (2003); Binz et al. 2004, loc. cit. By such methods designed ankyrin repeat protein libraries having randomized ankyrin repeat modules and/or randomized capping modules can be constructed. For example, such libraries could accordingly be assembled based on a fixed N-terminal capping module (e.g. the N-terminal capping module of SEQ ID NO: 5, 6 or 7) or a randomized N-terminal capping module according to SEQ ID NO: 8, one or more randomized repeat modules according to the sequence motif of SEQ ID NO: 9, 10 or 11, and a fixed C-terminal capping module (e.g. the C-terminal capping module of SEQ ID NO: 12, 13 or 14) or a randomized C-terminal capping module according to SEQ ID NO: 15. Preferably, such libraries are assembled to not have any of the amino acids C, G, M, N (in front of a G residue) and P at randomized positions of repeat or capping modules. In addition, randomized repeat modules according to the sequence motif of SEQ ID NO: 9, 10 or 11 could be further randomized at position 10 and/or position 17; the randomized N-terminal capping module according to the sequence motif of SEQ ID NO: 8 could be further randomized at position 9; and the randomized C-terminal capping module according to the sequence motif of SEQ ID NO: 15 could be further randomized at positions 10 and/or 17.

Furthermore, such randomized modules in such libraries may comprise additional polypeptide loop insertions with randomized amino acid positions. Examples of such polypeptide loop insertions are complement determining region (CDR) loop libraries of antibodies or de novo generated peptide libraries. For example, such a loop insertion could be designed using the structure of the N-terminal ankyrin repeat domain of human ribonuclease L (Tanaka, N., Nakanishi, M, Kusakabe, Y, Goto, Y., Kitade, Y, Nakamura, K. T., EMBO J. 23 (30), 3929-3938, 2004) as guidance. In analogy to this ankyrin repeat domain where ten amino acids are inserted in the beta-turn present close to the boarder of two ankyrin repeats, ankyrin repeat proteins libraries may contain randomized loops (with fixed and randomized positions) of variable length (e.g. 1 to 20 amino acids) inserted in one or more beta-turns of an ankyrin repeat domain.

Any such N-terminal capping module of an ankyrin repeat protein library preferably possesses the RILLAA, RILLKA or RELLKA motif (e.g. present from position 21 to 26 in SEQ ID NO: 29) and any such C-terminal capping module of an ankyrin repeat protein library preferably possesses the KLN, KLA or KAA motif (e.g. present at the last three amino acids in SEQ ID NO: 29).

The design of such an ankyrin repeat protein library may be guided by known structures of an ankyrin repeat domain interacting with a target. Examples of such structures, identified by their Protein Data Bank (PDB) unique accession or identification codes (PDB-IDs), are 1WDY, 3V31, 3V30, 3V2X, 3V2O, 3UXG, 3TWQ-3TWX, 1N11, 1S70 and 2ZGD.

Examples of designed ankyrin repeat protein libraries, such as N2C and N3C designed ankyrin repeat protein libraries, have been described (U.S. Pat. No. 7,417,130; Binz et al. 2003, loc. cit.; Binz et al. 2004, loc. cit.). The digit in N2C and N3C describes the number of randomized repeat modules present between the N-terminal and C-terminal capping modules.

The nomenclature used to define the positions inside the repeat units and modules is based on Binz et al. 2004, loc. cit. with the modification that borders of the ankyrin repeat modules and ankyrin repeat units are shifted by one amino acid position. For example, position 1 of an ankyrin repeat module of Binz et al. 2004 (loc. cit.) corresponds to position 2 of an ankyrin repeat module of the current disclosure and consequently position 33 of an ankyrin repeat module of Binz et al. 2004, loc. cit. corresponds to position 1 of a following ankyrin repeat module of the current disclosure.

### Example 1: Selection of Binding Proteins Comprising an Ankyrin Repeat Domain With Binding Specificity for CD40

Using ribosome display (Hanes, J. and Plückthun, A., PNAS 94, 4937-42, 1997), many ankyrin repeat proteins with binding specificity for human CD40 (hCD40) were selected from DARPin® libraries similar as described by Binz et al. 2004 (loc. cit.). The binding of the selected clones toward recombinant human CD40 target was assessed by crude extract Homogeneous Time Resolved Fluorescence (HTRF), indicating that hundreds of hCD40-specific binding proteins were successfully selected. For example, the ankyrin repeat domains of SEQ ID NO: 16 to 35 constitute amino acid sequences of selected binding proteins comprising an ankyrin repeat domain with binding specificity for hCD40. Individual ankyrin repeat modules from such ankyrin repeat domains with binding specificity to hCD40 are provided, e.g., in SEQ ID NO: 39 to 95.

### Selection of CD40-Specific Ankyrin Repeat Proteins by Ribosome Display

The selection of hCD40-specific ankyrin repeat proteins was performed by ribosome display (Hanes and Plückthun, loc. cit.) using the extracellular domain of human CD40 (SEQ ID NO: 97), fused at the C-terminal end to an IgG1 Fc domain via a short linker (amino acid sequence: AAA), as target protein, libraries of ankyrin repeat proteins as described above, and established protocols (See, e.g., Zahnd, C., Amstutz, P. and Plückthun, A., Nat. Methods 4, 69-79, 2007). The number of reverse transcription (RT)-PCR cycles after each selection round was constantly reduced, adjusting to the yield due to enrichment of binders. The first four rounds of selection employed standard ribosome display selection, using decreasing target concentration and increasing washing stringency to increase selection pressure from round 1 to round 4 (Binz et al. 2004, loc. cit.). For some pools, epitope blocking was used in some of the ribosome display selection rounds. To enrich high affinity CD40-specific ankyrin repeat proteins, the output from the fourth round of standard ribosome display selection (above) was subjected to an off-rate selection round with increased selection stringency (Zahnd, 2007, loc. cit.). A final standard selection round was performed after the off-rate selection round to amplify and recover the off-rate selected binding proteins. In these last two selection rounds, the number of RT-PCR cycles was kept constant. All pools of round 4 and round 6 were also formatted with a FAP-specific binding domain (see Example 3) at the N-terminus (i.e. resulting in a FC format) and one pool of round 4 was also formatted with the FAP-specific binding domain at the C-terminus (i.e. resulting in a CF format), to allow for functional screening in a reporter assay for activation of CD40 signaling in cells, which required clustering via FAP binding. In brief, the reporter assay used HEK293 cells expressing CD40 and harboring an NF-κB-dependent luciferase reporter gene. Following activation of CD40 signaling by human CD40 ligand or any other CD40 agonist, NF-κB transcription factors bind to the DNA response element to induce transcription of the luciferase gene in the reporter cells. These HEK293 cells were cultured in the presence and absence of FAP-expressing cells. CD40 signaling was activated by a CD40-specific binding protein of the invention if the binding protein was formatted with a FAP-specific binding domain (e.g. FC format) and only in the presence of FAP-expressing cells, leading to CD40 clustering upon binding of the FC formatted construct to CD40 and FAP. The FAP-specific binding domain is an example of a localizer molecule (see Example 3).

### Selected Clones Bind Specifically to CD40 as Shown by Crude Extract HTRF

Individual selected ankyrin repeat proteins specifically binding CD40 in solution were identified by a Homogeneous Time Resolved Fluorescence (HTRF) assay using crude extracts of ankyrin repeat protein-expressing Escherichia coli cells using standard protocols. Ankyrin repeat protein clones selected by ribosome display were cloned into a derivative of the pQE30 (Qiagen) expression vector, providing an N-terminal His-tag (SEQ ID NO: 4) to facilitate simple protein purification as described below, transformed into E. coli XL1-Blue (Stratagene), plated on LB-agar (containing 1% glucose and 50 μg/ml ampicillin) and then incubated overnight at 37° C. Single colonies were picked into a 96 well plate (each clone in a single well) containing 160 μl growth medium (TB containing 1% glucose and 50 μg/ml ampicillin) and incubated overnight at 37° C., shaking at 800 rpm. 150 μl of fresh TB medium containing 50 μg/ml ampicillin was inoculated with 8.5 μl of the overnight culture in a fresh 96-deep-well plate. After incubation for 120 minutes at 37° C. and 850 rpm, expression was induced with IPTG (0.5 mM final concentration) and continued for 4 hours. Cells were harvested and the pellets were frozen at −20° C. overnight before resuspension in 8.5 μl μl B-PERII (Thermo Scientific) and incubation for one hour at room temperature with shaking (600 rpm). Then, 160 μl PBS was added and cell debris was removed by centrifugation (3220 g for 15 min).

The extract of each lysed clone was applied as a 1:500 dilution (final concentration) in PBSTB (PBS supplemented with 0.1% Tween 200 and 0.2% (w/v) BSA, pH 7.4) together with 1.25 nM (final concentration) biotinylated human CD40, 1:400 (final concentration) of anti-6His-D2 HTRF antibody—FRET acceptor conjugate (Cisbio) and 1:400 (final concentration) of anti-strep-Tb antibody FRET donor conjugate (Cisbio) to a well of 384 well plate and incubated for 120 minutes at RT. The HTRF was read-out on a Tecan M1000 using a 340 nm excitation wavelength and a 665±10 nm emission filter. Screening of several hundred clones by such a crude cell extract HTRF revealed more than hundred different ankyrin repeat domains with specificity for human CD40. Examples of amino acid sequences of selected ankyrin repeat domains that specifically bind to human CD40 are provided in SEQ ID NOs: 16 to 35.

All of these ankyrin repeat domains (SEQ ID NOs: 16 to 35) (in FC format) also activated CD40 signaling in cells, as shown in the reporter assay, with EC50 values in the low nanomolar range (see Table 1).

TABLE 1
EC50 values of CD40-specific ankyrin repeat
domains in FC format in a reporter assay

| SEQ ID NO | EC50 [nM] |
|---|---|
| 16 | 9.0 |
| 17 | 13.7 |
| 18 | 8.5 |
| 19 | 12.2 |
| 20 | 5.1 |
| 21 | 3.0 |
| 22 | 2.8 |
| 23 | 4.1 |
| 24 | 1.5 |
| 25 | 10.8 |
| 26 | 7.5 |
| 27 | 12.0 |
| 28 | 0.4 |
| 29 | 5.0 |
| 30 | 26.9 |
| 31 | 5.3 |
| 32 | 5.6 |
| 33 | 1.2 |
| 34 | 4.2 |
| 35 | 3.1 |

### High level and Soluble Expression of CD40-Specific Ankyrin Repeat Proteins

For further analysis, the selected clones showing specific CD40 binding in the crude cell extract HTRF as described above were expressed in E. coli cells and purified using their His-tag according to standard protocols. 25 ml of stationary overnight cultures (TB, 1% glucose, 50 mg/l of ampicillin; 37° C.) were used to inoculate 500 ml cultures (TB, 50 mg/l ampicillin, 37° C.). At an absorbance of 1.0 to 1.5 at 600 nm, the cultures were induced with 0.5 mM IPTG and incubated at 37° C. for 4-5 h while shaking. The cultures were centrifuged and the resulting pellets were re-suspended in 25 ml of TBS₅₀₀ (50 mM Tris-HCl, 500 mM NaCl, pH 8) and lysed (sonication or French press). Following the lysis, the samples were mixed with 50 KU DNase/ml and incubated for 15 minutes prior to a heat-treatment step for 30 minutes at 62.5 ° C., centrifuged and the supernatant was collected and filtrated. Triton X100 (1% (v/v) final concentration) and imidazole (20 mM final concentration) were added to the homogenate. Proteins were purified over a Ni-nitrilotriacetic (Ni-NTA) acid column followed by a size exclusion chromatography on an ÄKTAxpress™ system according to standard protocols and resins known to the person skilled in the art. Alternatively, selected ankyrin repeat domains devoid of a His-tag are produced by high cell density fermentation in E. coli and purified by a series of chromatography and ultra/diafiltration steps according to standard resins and protocols known to the person skilled in the art. Highly soluble ankyrin repeat proteins with binding specificity for CD40 were purified from E. coli culture (up to 200 mg ankyrin repeat protein per liter of culture) with a purity>95% as estimated from 4-12% SDS-PAGE.

### Example 2: Determination of Dissociation Constants (K_{D}) of Ankyrin Repeat Proteins With Binding Specificity for CD40 by Surface Plasmon Resonance (SPR) Analysis

The binding affinities of the purified ankyrin repeat proteins on the human CD40 target were analyzed using a ProteOn instrument (BioRad) and the measurement was performed according standard procedures known to the person skilled in the art.

Briefly, biotinylated human CD40 was diluted in PBST (PBS, pH 7.4 containing 0.005% Tween 20®) and coated on two lanes of a NLC chip (BioRad) to levels of respectively 400 and 700 resonance units (RU). The interaction of ankyrin repeat protein and hCD40 was then measured by injecting 200 μl running buffer (PBS, pH 7.4 containing 0.005% Tween 20®) containing serial dilutions of ankyrin repeat proteins covering a concentration range between 50 nM and 3 nM for multi-trace SPR measurements (on-rate measurement), followed by a running buffer flow for at least 10 minutes at a constant flow rate of 100 μl/min (off-rate measurement). The regeneration was performed using 30 μl of 10 mM Glycine-HCl pH 2. The signals (i.e. resonance unit (RU) values) of the interspots and a reference injection (i.e. injection of running buffer only) were subtracted from the RU traces obtained after injection of ankyrin repeat protein (double-referencing). Based on the SPR traces obtained from the on-rate and off-rate measurements, the on- and off-rate of the corresponding ankyrin repeat protein—CD40 interaction was determined using a 1:1 Langmuir kinetic model.

Dissociation constants (K_{D}) were calculated from the estimated on- and off-rates using standard procedures known to the person skilled in the art. K_{D} values of the binding interactions of selected ankyrin repeat proteins with human CD40 were determined to be in the nanomolar range. Table 2 provides the K_{D} values of some selected ankyrin repeat proteins as examples.

TABLE 2
K_{D} values of ankyrin repeat protein - human CD40 interactions

| SEQ ID NO | K_{D} [nM] |
|---|---|
| 22 | 12.0 |
| 29 | 73.0 |
| 31 | 73.5 |

### Example 3: CD40-Specific Binding Proteins Combined With A Localizer Molecule

CD40-specific binding proteins were combined with a localizer molecule, and in some cases additionally with a serum half-life extending molecule, to evaluate if the CD40 agonist function of the CD40-specific binding proteins of the invention can be formatted in a multi-functional molecule in a manner that makes the CD40 agonist function effective and localizer-dependent and in addition may also provide serum half-life extension necessary for clinical development.

As a localizer molecule, a FAP-specific binding domain was chosen. SEQ ID NO: 98 provides a FAP-specific binding domain. As a half-life extending molecule, a serum albumin-specific binding domain was chosen. Such serum albumin-specific binding domains are known in the art.

Multi-functional molecules in various formats were generated and their FAP-specificity, efficacy and potency of CD40 activation were determined. These multi-functional proteins all comprised a localizer (i.e. the FAP-specific binding domain) and a CD40-specific binding domain. The impact of (i) adding human serum albumin (HSA)-binding domain(s), (ii) increasing valency by adding further CD40-binding domain(s), and (iii) changing the order of the binding domains within the protein were evaluated.

To compare the different formats, an in vitro assay was set up measuring the upregulation of the co-stimulatory receptor, CD86, expressed on human B cells upon CD40 triggering. This cellular assay used primary human B cells in the presence or absence of FAP-expressing cells. The upregulation of CD86 co-stimulatory molecule was evaluated as a marker of B cell activation. An anti-CD40 monoclonal antibody, whose mechanism of action is independent on FAP-mediated cross-linking, was used as reference material.

Multifunctional proteins of different formats. The simplest format of a multi-functional protein was a combination of one CD40-specific binding domain (SEQ ID NO: 29) and one localizer molecule (i.e. FAP-specific binding domain) (resulting in SEQ ID NO: 99; SMA014). Based on this initial format as parental molecule, several other multi-functional protein formats were generated, as summarized in Table 3.

TABLE 3
Multi-functional proteins in various domain formats

| SEQ ID NO / Construct Name | Format |
|---|---|
| SEQ ID NO: 99 / SMA014 | FC |
| SEQ ID NO: 100 / SMA087 | HFC |
| SEQ ID NO: 101 / SMA095 | HFCH |
| SEQ ID NO: 102 / SMA104 | FCC |
| SEQ ID NO: 103 / SMA091 | HFCC |
| SEQ ID NO: 104 / SMA099 | HFCCH |
| SEQ ID NO: 105 / AS579 | HHFCC |
| SEQ ID NO: 106 / SMA105 | FCCC |

“C”, “F”, and “H” in Table 3 indicate ankyrin repeat domains specifically binding CD40, FAP and HSA, respectively. The order of the different domains as indicated in Table 3 reflects the actual sequence of the domains from N-terminus to C-terminus in the molecular structure of the proteins. All the proteins additionally had a His-tag (SEQ ID NO: 4) at the N-terminus for ease of purification.

### Materials and Methods

As a reference, a CD40 monoclonal antibody was used. Binding of this CD40 mAb (an IgG2 mAb) to CD40 leads to the activation of antigen presenting cells independently of FAP. The anti-CD40 mAb corresponds to sequence 21.4.1 of U.S. Pat. No. 7,338,660 B2.

CHO cells were cultured at 37° C., 5% CO₂ in DMEM media containing 10% FBS and splitted every 2-3 days using accutase to detach cells.

The FAP-expressing CHO cell line is a stably transfected clonal cell line expressing human FAP on the cell surface. A plasmid containing a GFP-fusion of the ORF of human FAP was obtained from OriGene Technologies (#RG204692). The cDNA coding for human FAP (without GFP) was sub-cloned using standard molecular biology techniques. This plasmid was then transfected into CHO cells to produce stable transfectants overexpressing human FAP using Lipofectamine. Selection pressure was applied using different concentrations of Geneticin G-418 (Promega, V8091). Expression of FAP was analyzed by flow cytometry using an anti-FAP antibody corresponding to ESC11 (WO2011/040972). The population of FAP-CHO transfectants from condition 1.9 mg/mL G-418 (FAP-CHO-1.9) showed a lower expression level of FAP and those from condition 1.7 mg/mL (FAP-CHO-1.7) showed a higher expression level of FAP. The data in this Example were generated using FAP-CHO-1.7.

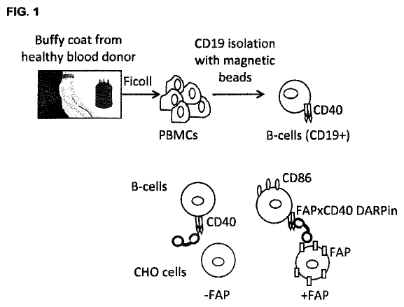

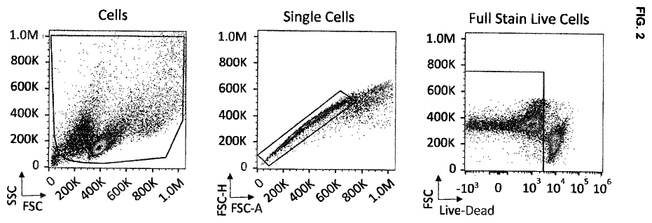

In vitro B cell activation assay. The design of the in vitro B cell activation assay is schematically shown in FIG. 1. Buffy coats were obtained from the Zurich blood donation center and diluted with PBS. Peripheral blood mononuclear cells (PBMCs) were then isolated by density centrifugation using Leucosep tubes. After several washing steps, human CD19⁺ B cells were enriched from PBMCs using a positive selection (human CD19 MicroBeads Kit) according to the manufacturer's recommendations. CD19⁺ B cells at 1×10⁵/well and FAP-expressing CHO cells or CHO wildtype (WT-CHO) cells at 5×10⁴ cells/well were seeded together in RPMI 1640 media+10% FBS with or without 600 μM HSA into 96-well plates together with dose titrations (400, 200, 40, 8, 5, 1.6, 0.3, 0 nM) of the indicated molecules. Cultures were incubated for 24 hours at 37° C., 5% CO2 and the upregulation of CD86 and CD69 on CD20⁺ B cells was assessed by flow cytometry using AttuneNxT.

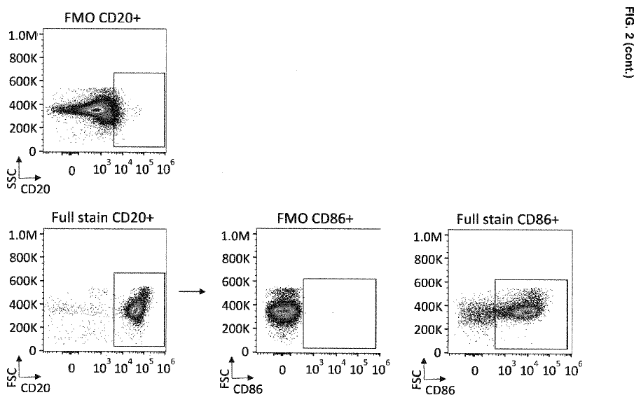

FACS staining, flow cytometer settings and antibody dilutions. Cells were first washed with 150 μl PBS and then incubated for 20 minutes at room temperature (RT) with 100 μl of BD human Fc-Block diluted (1:100) in PBS. After Fc-blocking incubation, the cells were incubated with 100 μl of directly labelled antibodies diluted (see Table 5 below for the dilution factors) in FACS buffer and incubated for another 20 minutes at 4° C. in the dark. Cells were washed with PBS, resuspended in 100 μl Live/Dead staining diluted (1:1000) in PBS and incubated for 20 minutes at 4° C. in the dark. One hundred μl of FACS buffer containing FBS reaction was added to stop the Live/Dead staining reaction. Cells were washed again with PBS and fixed using BD Cell Fix solution diluted (1:10) in water according to the manufacturer's recommendations. Dilutions of antibodies and FACS settings are summarized in Table 4 below. Compensation of the FACS machine was done with compensation beads according to the manufacturer's recommendations (ThermoFisher; AbC™ Total Antibody Compensation Bead Kit). Raw_fcs files were analyzed using FlowJo software (version 10.0.3). Cells were gated on live cells using Live-Dead discriminating dye followed by gating on CD20 positive cells as shown in FIG. 2 for CD86. The MFI and percentage of positive cells for CD86 were exported and plotted using GraphPad prism software, version 8.1.2.

TABLE 4
FSC: 200 SSC: 400 Acquisition: 200 ul/min, 100.000 events

| | | | Dilution | | |
|---|---|---|---|---|---|
| Target | Fluorochrome | Dilution | media | Channel | Voltages |
| CD20 | APC-Cy7 | 1:100 | FACS buffer | BL2 | 400 |
| CD86 | PE | 1:200 | FACS buffer | YL1 | 380 |
| CD69 | APC | 1:200 | FACS buffer | RL-1 | 400 |
| Live/Dead | Aqua | 1:1000 | PBS | VL2 | 400 |

EC50 Determinations. EC50 values were determined using GraphPad Prism version 7.02 by converting the x values (concentrations) in a log mode and fitting in a non-linear mode log (agonist) vs. response with a variable slope (three parameter) equation for determination of EC50 values.

Efficacy Determination. Efficacy values were determined using GraphPad Prism version 7.02 by calculating the average of duplicates for the MFI values at the highest concentration (400 nM).

### Results

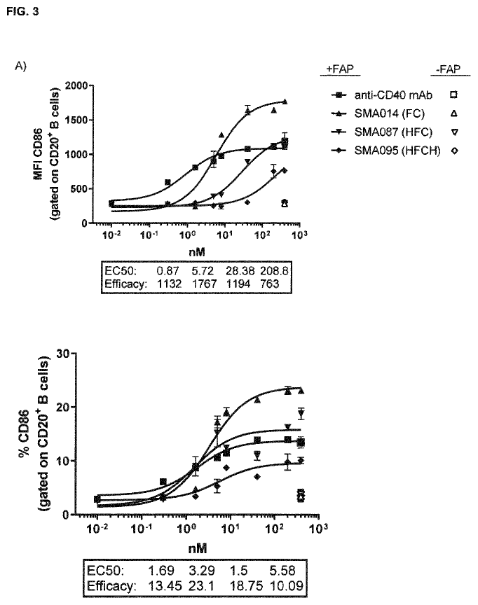

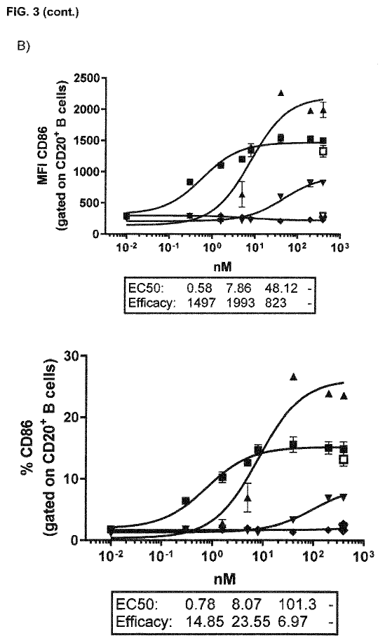

CD40-specific binding proteins of the invention can be combined with a localizer to generate a localizer-dependent CD40 agonist. A multi-functional molecule in the F-C format, SMA014, was tested in the in vitro human B cell activation assay. As shown in FIGS. 3, 4 and 5, this combination of a CD40-specific binding protein (SEQ ID NO: 29) with a localizer (a FAP-specific binding domain) resulted in a molecule that effectively activated CD40 signaling in human B cells in a strictly localizer-dependent (i.e. FAP-dependent) fashion. Only in the presence of FAP-expressing CHO cells (FAP-CHO), SMA014 effectively activated CD40 signaling in the B cells (full triangle pointing up symbols). In the presence of non-FAP expressing CHO cells (WT-CHO), SMA014 had no effect on CD40 signaling (empty triangle pointing up symbol). In contrast, and as expected, the agonistic anti-CD40 mAb induced activation of human B-cells independently of FAP expression, activating B-cells in the presence of either FAP-CHO or WT-CHO cells. Furthermore, SMA014 and the anti-CD40 mAb upregulated CD86 in a dose dependent manner. EC50 (potency) and maximum MFI (efficacy) mean values of two independent experiments are summarized in Table 5 and Table 6 for FAP-CHO and in Table 7 for WT-CHO, respectively.

In summary, these data demonstrated that the CD40 binding proteins of the invention can be combined with a localizer molecule to generate a multi-functional molecule which functions as an effective CD40 agonist in a strictly localizer-dependent mode of action.

HSA binding domain(s) impair potency and efficacy of the CD40-localizer binding protein (F-C format). The CD40 and localizer binding protein SMA014 was cloned in additional formats adding one (H-F-C, SMA087) or two (H-F-C-H, SMA095) HSA-binding ankyrin repeat domains which represent serum half-life extending moieties. These constructs were also tested in the in vitro B cell activation assay. As shown in FIG. 3, the HSA binding domain(s) impaired both the potency and the efficacy of the original multi-functional binding protein in the F-C format and the level of impairment correlated with the number of HSA binding domains added to the binding protein. Importantly, the inhibition was more pronounced in presence of 600 μM of albumin mimicking the physiological concentration of albumin in the human serum. It is plausible to hypothesize that the complex HSA binder/albumin could be a steric impairment for the binding, and consequently the activity, of the CD40 binding domain and/or the localizer. In absence and presence of HSA, HSA binding domains did not influence the FAP specific mode of action of the multi-functional CD40-localizer binding proteins. EC50 (potency) and maximum MFI (efficacy) mean values of two independent experiments are summarized in Table 5 and Table 6 for FAP-CHO and in Table 7 for WT-CHO, respectively.

In summary, the addition of half-life extending HSA-binding domain(s) impaired the functionality of the CD40-localizer binding protein (F-C format), the inhibition increased with the number of added HSA binding domains, and the inhibition was more pronounced in presence of physiological concentration of albumin.

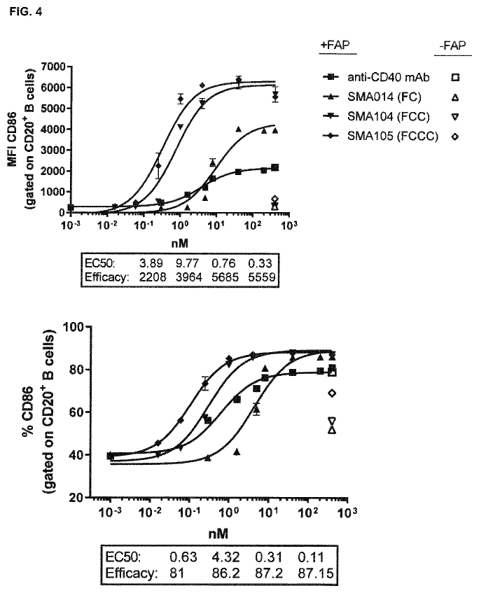

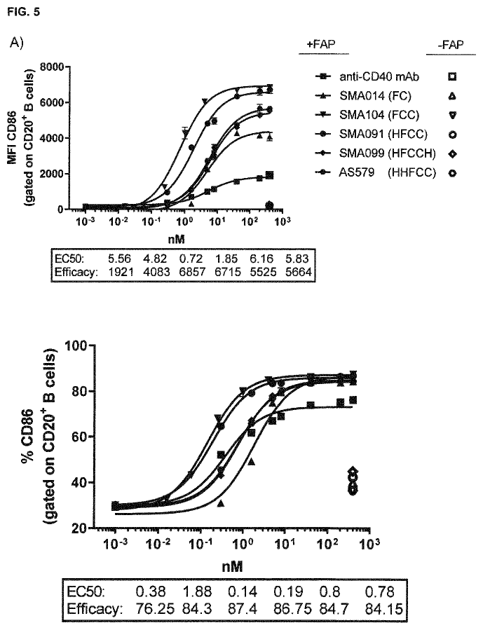

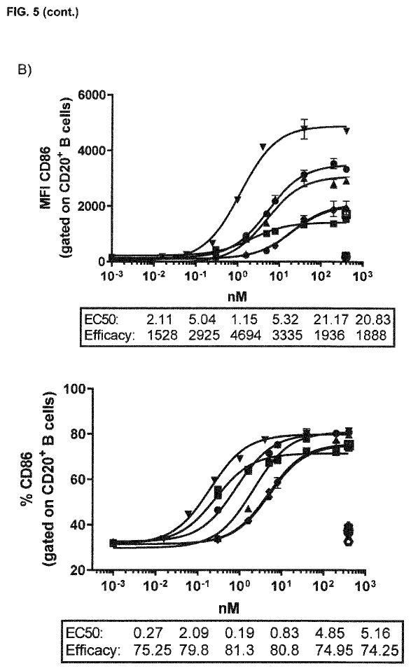

Bivalency for CD40 increases potency and efficacy and rescues the inhibitory effect induced by HSA-binding domain. Applicant then investigated how CD40 valency affects the performance of the multi-functional CD40 and localizer binding molecules. SMA014 was cloned in additional different formats adding one (F-C-C, SMA104) or two (F-C-C-C, SMA105) CD40-binding domains, and tested in the in vitro B-cell activation assay. As shown in FIG. 4, the bivalent and trivalent formats induced a stronger upregulation of CD86, indicating that valency contributes favorably to the performance of the molecule. Specifically, CD40 bivalency (SMA104) strongly increased potency (20×) and efficacy (2×) of the molecule in presence of FAP expressing CHO cells. CD40 trivalency (SMA105) only caused slightly increased potency of the molecule compared to CD40 bivalency, but did not further impact the efficacy. In absence of FAP, the bivalent CD40 format (SMA104) did not induce upregulation of CD86 on human B cells, while the trivalent CD40 format (SMA105) showed slight activation also in absence of FAP at the highest concentration, suggesting a possible FAP-independent activation induced by the trivalent CD40 binder. In view of these results, the bivalent CD40 format represents a preferred format. In particular, Applicant tested if the CD40 bivalency could rescue the inhibitory effect of the HSA binding domain. To address this question, the bivalent CD40 construct SMA104 was cloned with additional HSA binding domain(s) in different positions (clones SMA091, SMA099 and AS579; see Table 3 above for information on their domain formats). All the tested formats showed improved potency and efficacy compared to SMA014 (FIG. 5A). Importantly, in a more physiological condition in presence of HSA, activity of all formats with HSA binding domain was reduced, but SMA091 still showed improved activity compared to SMA014 (FIG. 5B). None of the multi-functional binding proteins enhanced expression of CD86 on the B cells in absence of FAP even at highest concentration (FIGS. 5A and B). As expected, the agonistic anti-CD40 mAb induced activation of human B-cells independently on FAP expression, activating B-cells either with FAP-CHO or WT-CHO. EC50 (potency) and maximum MFI (efficacy) mean values of two independent experiments are summarized in Table 5 and Table 6 for FAP-CHO and in Table 7 for WT-CHO, respectively.

TABLE 5

| EC50 [nM] | |
|---|---|
| MFI CD86 | % CD86 |

| Protein | w/o HSA | w HSA | w/o HSA | w HSA |
|---|---|---|---|---|
| SMA014 | 8.46 | 7.7 | 4.21 | 6.15 |
| SMA087 | 28.92 | 52.01 | 11.44 | 72.34 |
| SMA095 | 287.55 | — | 89.84 | — |
| CD40 mAb | 1.13 | 0.51 | 1.9 | 0.88 |
| SMA091 | 3.93 | 8.05 | 0.76 | 2.98 |
| SMA099 | 18.23 | 19.41 | 4.68 | 7.5 |
| SMA104 | 0.74 | | 0.23 | |
| SMA105 | 0.33 | | 0.11 | |
| AS579 | 10.01 | 23.1 | 2.27 | 10.25 |
| CD40 mAb | 4.73 | 2.05 | 0.51 | 0.3 |

TABLE 6

| Efficacy | |
|---|---|
| [MFI at highest dose] | |
| MFI CD86 | % CD86 |

| Protein | w/o HSA | w HSA | w/o HSA | w HSA |
|---|---|---|---|---|
| SMA014 | 2436.25 | 2189.75 | 48.4 | 47 |
| SMA087 | 1643.25 | 799.25 | 43.4 | 27.01 |
| SMA095 | 773 | 232.25 | 28.7 | — |
| CD40 mAb | 1339.5 | 1528.5 | 38.6 | 45.05 |
| SMA091 | 6230.25 | 2983.25 | 78.15 | 75.63 |
| SMA099 | 4954 | 1718.75 | 84.4 | 71.68 |
| SMA104 | 6270.75 | | 86.8 | |
| SMA105 | 5558.5 | | 87.15 | |
| AS579 | 4864.5 | 1781.75 | 82.98 | 71.9 |
| CD40 mAb | 2064.25 | 1867.25 | 78.63 | 76.63 |

TABLE 7

| Efficacy | |
|---|---|
| [MFI at highest dose] | |
| MFI CD86 | % CD86 |

| Protein | w/o HSA | w HSA | w/o HSA | w HSA |
|---|---|---|---|---|
| SMA014 | 293.75 | 310 | 13.15 | 13.29 |
| SMA087 | 291 | 299 | 12.53 | 13.73 |
| SMA095 | 299 | 306.5 | 12.48 | 13.68 |
| CD40 mAb | 1389.5 | 1473 | 38.83 | 40.05 |
| SMA091 | 289.5 | 295.5 | 47.78 | 43.13 |
| SMA099 | 289.25 | 273.75 | 47.38 | 42.53 |
| SMA104 | 251.75 | | 46.05 | |
| SMA105 | 672.5 | | 69.25 | |
| AS579 | 252.5 | 242.5 | 42.05 | 40.13 |
| CD40 mAb | 2053.75 | 1902 | 79 | 77.83 |

Conclusions: These data demonstrated that the CD40 binding proteins of the invention can be combined with a localizer molecule to generate a multi-functional molecule which functions as an effective CD40 agonist in a strictly localizer-dependent mode of action. The multi-functional CD40 and localizer (FAP) binding protein in the F-C format showed good biological activity in functional cell assays and good physical properties. However, this binding protein may require a half-life extension domain to allow its clinical development. Thus, different formats were analyzed to determine if and how the number and location of half-life extension HSA-binding domains impacts the activity of the molecule. It was observed that a half-life extension domain had a detrimental effect on the activity of the molecule and that this effect increased with the number of half-extension domains and the presence of HSA. Furthermore, it was then surprisingly found that CD40 bivalency (by having two CD40 binding domains) strongly increased the potency (20×) and the efficacy (2×) of the binding protein and maintained a stringent FAP-specific mechanism of action, but that CD40 trivalency (by having three CD40 binding domains) increased only slightly the potency compared to CD40 bivalency and did not show any further impact on the efficacy. In addition, the trivalent CD40 binding protein showed slight activation also in absence of FAP at the highest concentration, suggesting a partial loss of FAP-specific mode of action. CD40 bivalency was further found to be able to rescue the inhibitory effect of one half-life extension domain, by increasing the potency and the efficacy of the binding protein. Specifically, at physiological concentration of HSA, the binding protein in the H-F-C-C format retained an activity and FAP-specificity comparable to the binding protein in the F-C format. In conclusion, by adding a second CD40 binding domain we could prevent the detrimental effect of a HSA binding half-life extension domain and generate a molecule with similar functional properties to the parental binding protein in the F-C format but equipped with the half-life extension domain that will facilitate clinical development.

### Example 4: X-Ray Structure Analysis of a Complex of Human Tumor Necrosis Factor Receptor Superfamily Member 5 (hCD40) Bound by a CD40-Specific Binding Protein

The aim of this study was to generate and analyze complexes of recombinant hCD40 bound by a CD40-specific binding protein of the invention using X-ray crystallography. The CD40-specific binding protein used for this structural analysis was the DARPin® protein #29 (SEQ ID NO: 29).

### Materials and Methods

Protein production. hCD40 was expressed in Hi5 cells in the presence of tunicamycin to block glycosylation. Protein from culture supernatant was purified via HIS-Trap, THB-digest, negative HIS-Trap and SEC. Purified hCD40 was mixed in a 1:1.2 ratio with DARPin® protein #29. Excess of DARPin® protein #29 was removed from the hCD40: DARPin® protein #29 complex via SEC in 10 mM HEPES/NaOH pH 7, 150 mM NaCl. Sample was concentrated to 36.7 mg/ml. This procedure yielded homogenous protein with a purity greater than 95% as judged from Coomassie stained SDS-PAGE.

Crystallisation. The purified protein was used in crystallisation trials employing both, a standard screen with approximately 1200 different conditions, as well as crystallisation conditions identified using literature data. Conditions initially obtained were optimised using standard strategies, systemically varying parameters critically influencing crystallisation, such as temperature, protein concentration, drop ratio, and others. These conditions were also refined by systematically varying pH or precipitant concentrations.

Final crystallisation condition:
30% w/v PEG 4K
0.24 M LiSO4
0.1 M Tris pH=8.50
0.35 M NaBr

Data collection and processing. Crystals were flash-frozen and measured at a temperature of 100 K. The X-ray diffraction data were collected from complex crystals of hCD40 bound to the ligand DARPin® protein #29 at the SWISS LIGHT SOURCE (SLS, Villigen, Switzerland) using cryogenic conditions. The crystals belong to space group C 2. Data were processed using the programmes autoPROC, XDS and autoPROC, AIMLESS. The data collection and processing statistics for DARPin® protein #29 are listed in Table 8 below.

TABLE 8

| Ligand | DARPin hC23 | |
|---|---|---|
| X-ray source | PXII/X10SA (SLS¹) | |
| Wavelength [Å] | 0.9998 | |
| Detector | EIGER | |
| Temperature [K] | 100 | |
| Space group | C 2 | |
| Cell: a; b; c; [Å] | 193.67; 59.56; 81.84 | |
| α; β; γ; [ [symbol] ] | 90.0; 107.0; 90.0 | |
| Resolution [Å] | 2.29 | (2.33-2.29) |
| Unique reflections | 39800 | (1932) |
| Multiplicity | 4.3 | (4.4) |
| Completeness [%] | 97.8 | (97.3) |
| Rₚᵢₘ [%]⁶ | 4.3 | (61.8) |
| R_{sym} [%]³ | 7.8 | (114.9) |
| Rₘₑₐₛ [%]⁴ | 8.9 | (130.9) |
| CC1/2 [%] | 99.70 | (43.00) |
| Mean(I)/sd⁵ | 10.1 | (1.3) |

¹SWISS LEIGHT SOURCE (SLS, Villigen, Switzerland)
²values in parenthesis refer to the highest resolution bin.
${}^{3} Rsym = \frac{{\sum }_{h} {\sum }_{i}^{{n}_{h}} \vert {\hat{I}}_{h} - {I}_{h , i} \vert}{{\sum }_{h} {\sum }_{i}^{{n}_{h}} {I}_{h , i}} with {\hat{I}}_{h} = \frac{1}{{n}_{h}} {{\sum }^{{n}_{h}}}_{i} {I}_{h , i}$ where I_{h,i} is the intensity value of the ith measurement of h
${}^{4} Rmeas = \frac{{\sum }_{h} \sqrt{\frac{{n}_{h}}{{n}_{h} - 1}} {\sum }_{i}^{{n}_{h}} \vert {\hat{I}}_{h} - {I}_{h , i} \vert}{{\sum }_{h} {\sum }_{i}^{{n}_{h}} {I}_{h , i}} with {\hat{I}}_{h} = \frac{1}{{n}_{h}} {{\sum }^{{n}_{h}}}_{i} {I}_{h , i}$ where I_{h,i} is the intesity value of the ith measurement of h
⁵calculated from independent reflections
${}^{6} Precision ‐ indicating Rpim = \frac{{\sum }_{h} \sqrt{1 / ( N - 1} \vert {I}_{hi} - < {I}_{h} > \vert}{{\sum }_{h} < {I}_{h} >}$ [symbol] indicates data missing or illegible when filed

Structure modelling and refinement. The phase information necessary to determine and analyse the structure was obtained by molecular replacement. A previously solved structure of hCD40 was used as a search model. Subsequent model building and refinement was performed according to standard protocols with COOT and the software package CCP4, respectively. For calculation of the free R-factor, a measure to cross-validate the correctness of the final model, about 4.9% of measured reflections were excluded from the refinement procedure (see Table 9 below). TLS refinement (using REFMAC5, CCp4) was carried out, which resulted in lower R-factors and higher quality of the electron density map. Automatically generated local NCS restraints were applied (keyword “ncsr local” of newer REFMAC5 versions). The ligand parameterisation and generation of the corresponding library files were carried out with GRADE (Global Phasing Limited). The water model was built with the “Find waters”-algorithm of COOT by putting water molecules in peaks of the Fₒ-F_{c} map contoured at 3.0 with REFMAC5 and checking all waters with validation tool of COOT. The criteria for the list of suspicious waters were: B-factor greater 80 Å2, 2Fₒ-F_{c} map less than 1.2σ, distance to closest contact less than 2.3 Å, or more than 3.5 A. The suspicious water molecules and those in the ligand binding site (distance to ligand less than 10 Å) were checked manually. The Ramachandran Plot of the final model shows 92.2% of all residues in the most favoured region, 7.8% in the additionally allowed region, and 0.0% in the generously allowed region. No residues are found in the disallowed region (Table 9). Statistics of the final structure and the refinement process are listed in Table 9 below.

TABLE 9¹

| Ligand | DARPin hC23 |
|---|---|
| Resolution [Å] | 92.61-2.29 |
| Number of reflections (working /test) | 37835 / 1962 |
| R_{cryst} [%] | 21.9 |
| R_{free}[%]² | 25.1 |
| Total number of atoms: | |
| Protein | 4894 |
| Water | 208 |
| Sodium | 2 |
| Deviation from ideal geometry: ³ | |
| Bond lengths [Å] | 0.014 |
| Bond angles [°] | 1.59 |
| Bonded B's [Å²] ⁴ | 2.9 |
| Ramachandran plot: ⁵ | |
| Most favoured regions [%] | 92.2 |
| Additional allowed regions [%] | 7.8 |
| Generously allowed regions [%] | 0.0 |
| Disallowed regions [%] | 0.0 |

¹Values as defined in REFMAC5, without sigma cut-off
²Test-set contains 4.9% of measured reflections
³ Root mean square deviations from geometric target values
⁴ Calculated with MOLEMAN
⁵ Calculated with PROCHECK

### Results

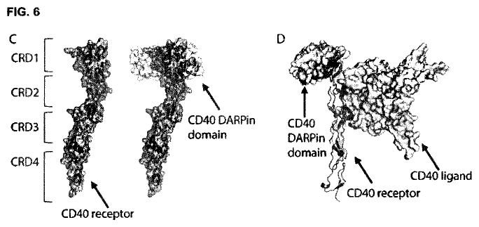

The structure was solved and refined to a final resolution of 2.29 Å. The structure analyses using X-ray crystallography revealed that DARPin® protein #29 bound to cysteine-rich domain (CRD) 1 (amino acids 23-59) of the CD40 receptor and that it bound to the CD40 receptor at one side opposite to the binding site of the CD40 ligand (CD40L), indicating the absence of direct binding site competition between the DARPin protein and the CD40L (FIGS. 6A and 6B). The CRD1 domain of the CD40 receptor is located distant from the cell membrane.

It has been reported that potent CD40 agonist antibodies bind membrane distal epitopes of the CD40 receptor (Yu et al., Cancer Cell 33, 664-675 e664 (2018)). Similarly, the X-ray crystallography study described in this Example showed that the CD40-specific binding protein of the invention interacts with the CRD1 of CD40 receptor, distant from the cell membrane. As it has already been suggested for CD40 agonist antibodies, a more cell membrane-distant epitope may lead to less steric hindrance, allowing better access to a CD40-specific binding protein of the invention comprising a localizer molecule and a more efficient clustering, and consequently a more efficient activation, of the CD40 receptor. Moreover, the region of interaction between the CD40-specific binding protein of the invention and CRD1 was shown to be opposite to the binding site of CD40L, suggesting the absence of direct binding competition between the binding protein of the invention and the CD40L. A compound that does not compete for CD40L may have an additive or synergistic effect with the ligand, resulting in a better activation of the receptor (see, e.g., Yu et al., loc. cit.; Challa et al., Allergy 54, 576-583 (1999); Pound et al., Int Immunol 11, 11-20 (1999)).

The specification is most thoroughly understood in light of the teachings of the references cited within the specification. The embodiments within the specification provide an illustration of embodiments of the invention and should not be construed to limit the scope of the invention. The skilled artisan readily recognizes that many other embodiments are encompassed by the invention. All publications, patents, and GenBank sequences cited in this disclosure are incorporated by reference in their entirety. To the extent the material incorporated by reference contradicts or is inconsistent with this specification, the specification will supersede any such material. The citation of any references herein is not an admission that such references are prior art to the present invention.

Those skilled in the art will recognize, or be able to ascertain using no more than routine experimentation, many equivalents to the specific embodiments of the invention described herein. Such equivalents are intended to be encompassed by the following embodiments.

1. A recombinant binding protein comprising an ankyrin repeat domain, wherein said ankyrin repeat domain has binding specificity for CD40, and wherein said ankyrin repeat domain comprises an ankyrin repeat module comprising an amino acid sequence selected from the group consisting of (1) SEQ ID NOs: 39 to 95 and (2) sequences in which up to 10 amino acids in any of SEQ ID NOs: 39 to 95 are substituted by other amino acids.

2. The binding protein of claim 1, wherein said ankyrin repeat module comprises an amino acid sequence selected from the group consisting of (1) SEQ ID NOs: 76, 77, and 78 and (2) sequences in which up to 10 amino acids in any of SEQ ID NOs: 76, 77, and 78 are substituted by other amino acids.

3. The binding protein of claim 1, wherein said ankyrin repeat module is a first ankyrin repeat module and comprises an amino acid sequence selected from the group consisting of (1) SEQ ID NO: 76 and (2) sequences in which up to 10 amino acids in SEQ ID NO: 76 are substituted by other amino acids, and wherein said ankyrin repeat domain further comprises (i) a second ankyrin repeat module comprising an amino acid sequence selected from the group consisting of (1) SEQ ID NO: 77 and (2) sequences in which up to 10 amino acids in SEQ ID NO: 77 are substituted by other amino acids and (ii) a third ankyrin repeat module comprising an amino acid sequence selected from the group consisting of (1) SEQ ID NO: 78 and (2) sequences in which up to 10 amino acids in SEQ ID NO: 78 are substituted by other amino acids.

4. The binding protein of claim 3, wherein said first ankyrin repeat module is located N-terminally of said second ankyrin repeat module, and said second ankyrin repeat module is located N-terminally of said third ankyrin repeat module within said ankyrin repeat domain.

5. The binding protein of claim 1, wherein said ankyrin repeat domain further comprises an N-terminal capping module, wherein said N-terminal capping module comprises an amino acid sequence selected from the group consisting of (1) SEQ ID NOs: 5 to 8 and (2) sequences in which up to 10 amino acids in any of SEQ ID NOs: 5 to 7 are substituted by other amino acids.

6. The binding protein of claim 1, wherein said ankyrin repeat domain further comprises a C-terminal capping module, wherein said C-terminal capping module comprises an amino acid sequence selected from the group consisting of (1) SEQ ID NOs: 12 to 15 and (2) sequences in which up to 10 amino acids in any of SEQ ID NOs: 12 to 14 are substituted by other amino acids.

7. A recombinant binding protein comprising an ankyrin repeat domain, wherein said ankyrin repeat domain has binding specificity for CD40, and wherein said ankyrin repeat domain comprises an amino acid sequence with at least 80% amino acid sequence identity with any one of SEQ ID NOs: 16 to 35, wherein G at position 1 and/or S at position 2 of SEQ ID NOs: 16 to 35 are optionally missing.

8. The binding protein of claim 7, wherein said ankyrin repeat domain comprises an amino acid sequence with at least 90% amino acid sequence identity with SEQ ID NO: 29, wherein G at position 1 and/or S at position 2 of said ankyrin repeat domain are optionally missing.

9. The binding protein of claim 7, wherein said ankyrin repeat domain comprises the amino acid sequence of SEQ ID NO: 29, wherein G at position 1 and/or S at position 2 of said ankyrin repeat domain are optionally missing.

10. The binding protein of claim 7, wherein said ankyrin repeat domain binds human CD40 in PBS with a dissociation constant (K_{D}) below 10⁻⁷M.

11. The binding protein of claim 7, wherein said ankyrin repeat domain specifically binds to the N-terminal cysteine-rich domain 1 (CRD1) (amino acids 23-59 of SEQ ID NO: 96) of the CD40 receptor.

12. The binding protein of claim 7, wherein said binding protein further comprises a second ankyrin repeat domain with binding specificity for CD40.

13. The binding protein of claim 12, wherein said second ankyrin repeat domain specifically binds to the N-terminal CRD1 (amino acids 23-59 of SEQ ID NO: 96) of the CD40 receptor.

14. The binding protein of claim 7, wherein said binding protein further comprises a localizer molecule.

15. A nucleic acid encoding the binding protein or the ankyrin repeat domain of claim 7.

16. A pharmaceutical composition comprising the binding protein of claim 7, and optionally a pharmaceutically acceptable carrier and/or diluent.

17. A method of localized activation of CD40 in CD40-expressing cells in a mammal, including a human, the method comprising the step of administering to said mammal the binding protein of claim 7.

18. The method of claim 17, wherein said CD40-expressing cells are located in a tumor.

19. A method of treating a medical condition, the method comprising the step of administering to a patient in need thereof a therapeutically effective amount of the binding protein of claim 7.

20. The method of claim 19, wherein said medical condition is a cancer.


SEQUENCE LISTING (106 sequences)

SEQ ID NO: 1 - protein, 24 aa, Artificial Sequence
  PT-rich peptide linker
GSPTPTPTTPTPTPTTPTPTPTGS

SEQ ID NO: 2 - protein, 22 aa, Artificial Sequence
  PT-rich peptide linker
GSPTPTPTTPTPTPTTPTPTPT

SEQ ID NO: 3 - protein, 5 aa, Artificial Sequence
  Consensus GS linker
  VARIANT 1..5: [Gly-Gly-Gly-Gly-Ser]n, wherein n is 1, 2, 3, 4, 5, or 6
GGGGS

SEQ ID NO: 4 - protein, 10 aa, Artificial Sequence
  His-tag
MRGSHHHHHH

SEQ ID NO: 5 - protein, 32 aa, Artificial Sequence
  N-terminal capping module
GSDLGKKLLQAARAGQLDEVRELLKAGADVNA

SEQ ID NO: 6 - protein, 32 aa, Artificial Sequence
  N-terminal capping module
GSDLGKKLLQAARAGQLDEVRILLKAGADVNA

SEQ ID NO: 7 - protein, 32 aa, Artificial Sequence
  N-terminal capping module
GSDLGSKLLQAARAGQLDTVRTLLQAGADVNA

SEQ ID NO: 8 - protein, 32 aa, Artificial Sequence
  N-terminal capping module
  VARIANT 6: Xaa can be any naturally occuring amino acid
  VARIANT 7: Xaa can be any naturally occuring amino acid
  VARIANT 13: Xaa can be any naturally occuring amino acid
  VARIANT 14: Xaa can be any naturally occuring amino acid
  VARIANT 19: Xaa can be any naturally occuring amino acid
  VARIANT 22: Xaa can be any naturally occuring amino acid
  VARIANT 24: Xaa can be any naturally occuring amino acid
  VARIANT 25: Xaa can be any naturally occuring amino acid
  VARIANT 26: Xaa can be any naturally occuring amino acid
GSDLGXXLLQAAXXGQLDXVRXLXXXGADVNA

SEQ ID NO: 9 - protein, 33 aa, Artificial Sequence
  Ankyrin repeat module
  VARIANT 1: Xaa can be any occuring amino acid
  VARIANT 3: Xaa can be any occuring amino acid
  VARIANT 4: Xaa can be any occuring amino acid
  VARIANT 6: Xaa can be any occuring amino acid
  VARIANT 11: Xaa can be any occuring amino acid
  VARIANT 14: Xaa can be any occuring amino acid
  VARIANT 15: Xaa can be any occuring amino acid
  VARIANT 27: Xaa can be any occuring amino acid
XDXXGXTPLHXAAXXGHLEIVEVLLKXGADVNA

SEQ ID NO: 10 - protein, 33 aa, Artificial Sequence
  Ankyrin repeat module
  VARIANT 3: Xaa can be any occuring amino acid
  VARIANT 4: Xaa can be any occuring amino acid
  VARIANT 6: Xaa can be any occuring amino acid
  VARIANT 14: Xaa can be any occuring amino acid
  VARIANT 15: Xaa can be any occuring amino acid
KDXXGXTPLHLAAXXGHLEIVEVLLKAGADVNA

SEQ ID NO: 11 - protein, 33 aa, Artificial Sequence
  Ankyrin repeat module
  VARIANT 3: Xaa can be any occuring amino acid
  VARIANT 4: Xaa can be any occuring amino acid
  VARIANT 6: Xaa can be any occuring amino acid
  VARIANT 11: Xaa can be any occuring amino acid
  VARIANT 14: Xaa can be any occuring amino acid
  VARIANT 15: Xaa can be any occuring amino acid
KDXXGXTPLHXAAXXGHLEIVEVLLKAGADVNA

SEQ ID NO: 12 - protein, 28 aa, Artificial Sequence
  C-terminal capping module
QDKFGKTPADIAADNGHEDIAEVLQKLN

SEQ ID NO: 13 - protein, 28 aa, Artificial Sequence
  C-terminal capping module
QDKSGKTPADLAARAGHQDIAEVLQKAA

SEQ ID NO: 14 - protein, 28 aa, Artificial Sequence
  C-terminal capping module
QDTQGTTPADLAARAGHQQIASVLQQAA

SEQ ID NO: 15 - protein, 28 aa, Artificial Sequence
  C-terminal capping module
  VARIANT 1: Xaa can be any occuring amino acid
  VARIANT 3: Xaa can be any occuring amino acid
  VARIANT 4: Xaa can be any occuring amino acid
  VARIANT 6: Xaa can be any occuring amino acid
  VARIANT 11: Xaa can be any occuring amino acid
  VARIANT 15: Xaa can be any occuring amino acid
  VARIANT 19: Xaa can be any occuring amino acid
  VARIANT 22: Xaa can be any occuring amino acid
  VARIANT 26: Xaa can be any occuring amino acid
XDXXGXTPADXAARXGHQXIAXVLQXAA

SEQ ID NO: 16 - protein, 126 aa, Artificial Sequence
  Ankyrin repeat domain
GSDLGFKLLAAAFEGQDDEVRILLAAGADVNAKDKVGYTPLHYAAHAGHLEIVEVLLKAGADVNAKDVFGRTPLHSAALHGHLEIVEVLLKAGADVNAQDTRGRTPADLAAILGHEDIAEVLQKAA

SEQ ID NO: 17 - protein, 159 aa, Artificial Sequence
  Ankyrin repeat domain
GSDLGKKLLQAARAGQLDEVRELLKAGADVNAKDSEGSTPLHLAALKGHLEIVEVLLKAGADVNAKDWQGQTPLHLAAFEGHLEIVEVLLKAGADVNAKDRWGETPLHIAAKHGHLEIVEVLLKAGADVNAQDKSGKTPADLAARAGHRDIAEVLQKAA

SEQ ID NO: 18 - protein, 159 aa, Artificial Sequence
  Ankyrin repeat domain
GSDLGKKLLQAARAGQLDEVRILLATGADVNAKDEIGYTPLHWAAYEGHLEIVEVLLKAGADVNAKDEHGRTPLHLAAYKGHLEIVEVLLKAGADVNAKDSWGRTPLHIAARAGHLEIVEVLLKSGADVNAQDKSGKTPADLAARAGHQDIAEVLQKAA

SEQ ID NO: 19 - protein, 159 aa, Artificial Sequence
  Ankyrin repeat domain
GSDLGKKLLQAARAGQLDEVRELLKAGADVNAKDTIGWTPLHLAAYRGHLEIVEVLLKAGADVNAKDEHGRTPLHLAAHAGHLEIVEVLLKAGADVNAKDAWGHTPLHIAARTGHLEIVEVLLKAGADVNAQDKSGKTPADLAARAGHQDIAEVLQKAA

SEQ ID NO: 20 - protein, 159 aa, Artificial Sequence
  Ankyrin repeat domain
GSDLGKKLLQAARVGQLDEVRELLKAGADVNAKDQLGWTPLHLAAYEGHLEIVEVLLKAGADVNAKDEKGRTPLHLAAYKGHLEIVEVLLKAGADVNAKDSWGRTPLHIAARAGHLEIVEVLLKSGADVNAQDKSGKTPADLAARAGHQDIAEVLQKAA

SEQ ID NO: 21 - protein, 159 aa, Artificial Sequence
  Ankyrin repeat domain
GSDLGKKLLQAARAGQLDEVRELLKAGADVNAKDVWGWTPLHLAAFEGHLEIVEVLLKAGADVNAKDHHGRTPLHLAAQYGHLEIVEVLLKAGADVNAKDRYGQTPLHVAAEHGHLEIVEVLLKAGADVNAQDKSGKTPADLAARAGHQDIAEVLQKAA

SEQ ID NO: 22 - protein, 159 aa, Artificial Sequence
  Ankyrin repeat domain
GSDLGKKLLQAARAGQLDEVRELLKAGADVNAKDEFGQTPLHIAAKSGHLEIVEVLLKAGADVNAKDVYGRTPLHLAAQYGHLEIVEVLLKAGADVNAKDTQGVTPLHVAAFKGHLEIVEVLLKAGADVNAQDKSGKTPADLAARAGHQDIAEVLQKAA

SEQ ID NO: 23 - protein, 159 aa, Artificial Sequence
  Ankyrin repeat domain
GSDLGKKLLQAVRAGQLDEVRELLKAGADVNAKDTWGFTPLHIAAESGHLEIVEVLLKAGADVNAKDVQGRTPLHIAAHSGHLEIVEVLLKAGADVNAKDFRGWTPLHLAAWSGHLEIVEVLLKAGADVNAQDKSGKTPADLAARAGHQDIAEVLQKAA

SEQ ID NO: 24 - protein, 159 aa, Artificial Sequence
  Ankyrin repeat domain
GSDLGKKLLQAARAGQLDEVRELLKAGADVNAKDTWGFTPLHIAAESGHLEIVEVLLKAGADVNAKDVQGRTPLHIAAHSGHLEIVEVLLKAGADVNAKDFRGWTPLHLAAWSGHLEIVGVLLKAGADVNAQDKSGKTPADLAARAGHQDIAEVLQKAA

SEQ ID NO: 25 - protein, 159 aa, Artificial Sequence
  Ankyrin repeat domain
GSDLGKKLLQATRAGQLDEVRELLKAGADVNAKDEFGRTPLHLAAQYGHLEIVEVLLKAGADVNAKDAHGVTPLHVAAFRGHLEIVEVLLKAGADVNAKDRSGLTPLHLAAWQGHLEIVEVLLKAGADVNAQDKSGKTPADLAARAGHQDIAEVLQKAA

SEQ ID NO: 26 - protein, 159 aa, Artificial Sequence
  Ankyrin repeat domain
GSDLGKKLLQAARAGQLDEVRELLKAGADVNAKDTYGRTPLHIAAQYGHLEIVEVLLKAGADVNAKDHRGVTPLHVAAYKGHLEIVEVLLKAGADVNAKDVTGVTPLHVAAFKGHLEIVEVLLKAGADVNAQDKSGKTPADLTARAGHQDIAEVLQKAA

SEQ ID NO: 27 - protein, 126 aa, Artificial Sequence
  Ankyrin repeat domain
GSDLGYKLLWAAYAGQDDEVRILLVAGADVNAKDSYGRTPLHYAANAGHLEIVEVLLKAGADVNAKDIQGETPLHHAARKGHLEIVEVLLKAGADVNAQDKTGDTPADLAAFVGHEDIAEVLQKAA

SEQ ID NO: 28 - protein, 159 aa, Artificial Sequence
  Ankyrin repeat domain
GSALGKKLLQAARAGQLDEVRELLKAGADVNAKDQEGRTPLHLAARSGHLEIVEVLLKAGADVNAKDTFGITPLHLAALPRSPEIVEVLLKAGADVNAKDQFGRTPLHIAAQSGHLEIVEVLLKAGADVNAQDKSGKTPADLAARAGHQDIAEVLQKAA

SEQ ID NO: 29 - protein, 159 aa, Artificial Sequence
  Ankyrin repeat domain
GSDLGKKLLQAARAGQLDEVRELLKAGADVNAKDTWGFTPLHIAAESGHLEIVEVLLKAGADVNAKDVQGRTPLHIAAHSGHLEIVEVLLKAGADVNAKDFRGWTPLHLAAWSGHLEIVEILLKAGADVNAQDKSGKTPADLAARAGHQDIAEVLQKAA

SEQ ID NO: 30 - protein, 126 aa, Artificial Sequence
  Ankyrin repeat domain
GSDLGHKLLYAAYTGQDDEVRILLAAGADVNAKDSVGYTPLHYAAHAGHLEIVEVLLKAGADVNAKDVFGRTPLHSAALHGHLEIVEVLLKAGADVNAQDTRGRTPADLAAILGHEDIAEVLQKAA

SEQ ID NO: 31 - protein, 159 aa, Artificial Sequence
  Ankyrin repeat domain
GSDLGKKLLWAAAAGQLDEVRELLKAGADVNAKDHVGYTPLHIAALAGHLEIVEVLLKAGEDVNAKDHKGRTPLHVAAAVGHLEIVEVLLKAGADVNAKDQQGVTPLHIAAITGHLEIVEVLLKAGADVNAQDKSGKTPADLAARAGHQDIAEVLQKAA

SEQ ID NO: 32 - protein, 159 aa, Artificial Sequence
  Ankyrin repeat domain
GSDLGKKLLQAARAGQLDEVRELLKAGADVNAKDTWGFTPLHIAAESGHLEIVEVLLKAGADVNAKDVQGRTPLHIAAHSGHLEIVEVLLKVGADVNAKDFRGWTPLHLAAWSGHLEIVEVLLKAGADVNAQDKSGKTPADLAARAGHQDIAEVLQKAA

SEQ ID NO: 33 - protein, 159 aa, Artificial Sequence
  Ankyrin repeat domain
GSDLGKKLLQAARAGQLDEVRELLKAGADVNAKDTYGRTPLHIAAQYGHLEIVEVLLKAGADVNAKDHRGVTPLHVAAYKGHLEIVEVLLKAGADVNAKDVTGVTPLHVAAFKGHLEIVEVLLKAGADVNAQDKSGKTPADLAARAGHQDIAEVLQKAA

SEQ ID NO: 34 - protein, 159 aa, Artificial Sequence
  Ankyrin repeat domain
GSDLGKKLLQAARAGQLDEVRELLKAGADVNAKDEWGRTPLHLAAQYGHLEIVEVLLKAGADVNAKDHRGVTPLHVAAYKGHLEIVEVLLKAGADVNAKDVTGVTPLHVAAFKGHLEIVEVLLKAGADVNAQDKSGKTPADLAARAGHQDIAEVLQKAA

SEQ ID NO: 35 - protein, 159 aa, Artificial Sequence
  Ankyrin repeat domain
GSDLGKKLLQAARAGQLDEVRELLKAGADVNAKDTWGFTPLHIATESGHLEIVEVLLKAGADVNAKDVQGRTPLHIAAHSGHLEIVEVLLKAGADVNAKDFRGWTPLHLAAWSGHLEIVEVLLKAGADVNAQDKSGKTPADLAARAGHQDIAEVLQKAA

SEQ ID NO: 36 - DNA, 471 bp, Artificial Sequence
  Nucleic acid encoding an ankyrin repeat domain
gacctgggtaaaaaactgctgcaagcagcacgtgcaggtcagctggatgaagttcgtgaactgctgaaagcaggcgccgatgttaatgcaaaagatacctggggcttcaccccgctgcatattgctgctgagtctggtcacctggaaattgttgaagttctgctgaaagccggtgcagatgttaatgcaaaagatgtgcaaggcagaaccccgctgcatatcgctgctcactctggtcacctggaaattgttgaagttctgctgaaagccggtgcagatgttaatgcaaaagatttcagaggctggaccccgctgcatctggctgcttggtctggtcacctggaaattgttgaaattctgctgaaagccggtgcagatgttaacgcacaggataaaagcggtaaaacccctgccgatctggcagctcgcgccggtcatcaagatattgctgaagtgctgcagaaggcagca

SEQ ID NO: 37 - DNA, 471 bp, Artificial Sequence
  Nucleic acid encoding an ankyrin repeat domain
gacctgggtaaaaaactgctgcaagcagcacgtgcaggtcagctggatgaagttcgtgaactgctgaaagcaggcgccgatgttaatgcaaaagatgagttcggccaaaccccgctgcatatcgctgctaagtctggtcacctggaaattgttgaagttctgctgaaagccggtgcagatgttaatgcaaaagatgtgtacggcagaaccccgctgcatctggctgctcaatacggtcacctggaaattgttgaagttctgctgaaagccggtgcagatgttaatgcaaaagatacccaaggcgtgaccccgctgcacgtggctgctttcaagggtcacctggaaattgttgaagttctgctgaaagccggtgcagatgttaacgcacaggataaaagcggtaaaacccctgccgatctggcagctcgcgccggtcatcaagatattgctgaagtgctgcagaaggcagca

SEQ ID NO: 38 - DNA, 471 bp, Artificial Sequence
  Nucleic acid encoding an ankyrin repeat domain
gacctgggtaaaaaactgctgtgggctgctgctgcaggtcagctggatgaagttcgtgaactgctgaaagcaggcgccgatgttaatgcaaaagatcatgtgggctacaccccgctgcatatcgctgctctggctggtcacctggaaattgttgaagttctgctgaaagccggtgaagatgttaatgcaaaagatcacaagggcagaaccccgctgcatgtggctgctgctgtgggtcacctggaaattgttgaagttctgctgaaagccggtgcagatgttaatgcaaaagatcagcagggcgtgaccccgctgcatatcgctgctatcaccggtcacctggaaattgttgaagttctgctgaaagccggtgcagatgttaacgcacaggataaaagcggtaaaacccctgccgatctggcagctcgcgccggtcatcaagatattgctgaagtgctgcagaaggcagca

SEQ ID NO: 39 - protein, 33 aa, Artificial Sequence
  Ankyrin repeat module
KDKVGYTPLHYAAHAGHLEIVEVLLKAGADVNA

SEQ ID NO: 40 - protein, 33 aa, Artificial Sequence
  Ankyrin repeat module
KDVFGRTPLHSAALHGHLEIVEVLLKAGADVNA

SEQ ID NO: 41 - protein, 33 aa, Artificial Sequence
  Ankyrin repeat module
KDSEGSTPLHLAALKGHLEIVEVLLKAGADVNA

SEQ ID NO: 42 - protein, 33 aa, Artificial Sequence
  Ankyrin repeat module
KDWQGQTPLHLAAFEGHLEIVEVLLKAGADVNA

SEQ ID NO: 43 - protein, 33 aa, Artificial Sequence
  Ankyrin repeat module
KDRWGETPLHIAAKHGHLEIVEVLLKAGADVNA

SEQ ID NO: 44 - protein, 33 aa, Artificial Sequence
  Ankyrin repeat module
KDEIGYTPLHWAAYEGHLEIVEVLLKAGADVNA

SEQ ID NO: 45 - protein, 33 aa, Artificial Sequence
  Ankyrin repeat module
KDEHGRTPLHLAAYKGHLEIVEVLLKAGADVNA

SEQ ID NO: 46 - protein, 33 aa, Artificial Sequence
  Ankyrin repeat module
KDSWGRTPLHIAARAGHLEIVEVLLKSGADVNA

SEQ ID NO: 47 - protein, 33 aa, Artificial Sequence
  Ankyrin repeat module
KDTIGWTPLHLAAYRGHLEIVEVLLKAGADVNA

SEQ ID NO: 48 - protein, 33 aa, Artificial Sequence
  Ankyrin repeat module
KDEHGRTPLHLAAHAGHLEIVEVLLKAGADVNA

SEQ ID NO: 49 - protein, 33 aa, Artificial Sequence
  Ankyrin repeat module
KDAWGHTPLHIAARTGHLEIVEVLLKAGADVNA

SEQ ID NO: 50 - protein, 33 aa, Artificial Sequence
  Ankyrin repeat module
KDQLGWTPLHLAAYEGHLEIVEVLLKAGADVNA

SEQ ID NO: 51 - protein, 33 aa, Artificial Sequence
  Ankyrin repeat module
KDEKGRTPLHLAAYKGHLEIVEVLLKAGADVNA

SEQ ID NO: 52 - protein, 33 aa, Artificial Sequence; identical to SEQ ID NO: 46
  Ankyrin repeat module

SEQ ID NO: 53 - protein, 33 aa, Artificial Sequence
  Ankyrin repeat module
KDVWGWTPLHLAAFEGHLEIVEVLLKAGADVNA

SEQ ID NO: 54 - protein, 33 aa, Artificial Sequence
  Ankyrin repeat module
KDHHGRTPLHLAAQYGHLEIVEVLLKAGADVNA

SEQ ID NO: 55 - protein, 33 aa, Artificial Sequence
  Ankyrin repeat module
KDRYGQTPLHVAAEHGHLEIVEVLLKAGADVNA

SEQ ID NO: 56 - protein, 33 aa, Artificial Sequence
  Ankyrin repeat module
KDEFGQTPLHIAAKSGHLEIVEVLLKAGADVNA

SEQ ID NO: 57 - protein, 33 aa, Artificial Sequence
  Ankyrin repeat module
KDVYGRTPLHLAAQYGHLEIVEVLLKAGADVNA

SEQ ID NO: 58 - protein, 33 aa, Artificial Sequence
  Ankyrin repeat module
KDTQGVTPLHVAAFKGHLEIVEVLLKAGADVNA

SEQ ID NO: 59 - protein, 33 aa, Artificial Sequence
  Ankyrin repeat module
KDTWGFTPLHIAAESGHLEIVEVLLKAGADVNA

SEQ ID NO: 60 - protein, 33 aa, Artificial Sequence
  Ankyrin repeat module
KDVQGRTPLHIAAHSGHLEIVEVLLKAGADVNA

SEQ ID NO: 61 - protein, 33 aa, Artificial Sequence
  Ankyrin repeat module
KDFRGWTPLHLAAWSGHLEIVEVLLKAGADVNA

SEQ ID NO: 62 - protein, 33 aa, Artificial Sequence; identical to SEQ ID NO: 59
  Ankyrin repeat module

SEQ ID NO: 63 - protein, 33 aa, Artificial Sequence; identical to SEQ ID NO: 60
  Ankyrin repeat module

SEQ ID NO: 64 - protein, 33 aa, Artificial Sequence
  Ankyrin repeat module
KDFRGWTPLHLAAWSGHLEIVGVLLKAGADVNA

SEQ ID NO: 65 - protein, 33 aa, Artificial Sequence
  Ankyrin repeat module
KDEFGRTPLHLAAQYGHLEIVEVLLKAGADVNA

SEQ ID NO: 66 - protein, 33 aa, Artificial Sequence
  Ankyrin repeat module
KDAHGVTPLHVAAFRGHLEIVEVLLKAGADVNA

SEQ ID NO: 67 - protein, 33 aa, Artificial Sequence
  Ankyrin repeat module
KDRSGLTPLHLAAWQGHLEIVEVLLKAGADVNA

SEQ ID NO: 68 - protein, 33 aa, Artificial Sequence
  Ankyrin repeat module
KDTYGRTPLHIAAQYGHLEIVEVLLKAGADVNA

SEQ ID NO: 69 - protein, 33 aa, Artificial Sequence
  Ankyrin repeat module
KDHRGVTPLHVAAYKGHLEIVEVLLKAGADVNA

SEQ ID NO: 70 - protein, 33 aa, Artificial Sequence
  Ankyrin repeat module
KDVTGVTPLHVAAFKGHLEIVEVLLKAGADVNA

SEQ ID NO: 71 - protein, 33 aa, Artificial Sequence
  Ankyrin repeat module
KDSYGRTPLHYAANAGHLEIVEVLLKAGADVNA

SEQ ID NO: 72 - protein, 33 aa, Artificial Sequence
  Ankyrin repeat module
KDIQGETPLHHAARKGHLEIVEVLLKAGADVNA

SEQ ID NO: 73 - protein, 33 aa, Artificial Sequence
  Ankyrin repeat module
KDQEGRTPLHLAARSGHLEIVEVLLKAGADVNA

SEQ ID NO: 74 - protein, 33 aa, Artificial Sequence
  Ankyrin repeat module
KDTFGITPLHLAALPRSPEIVEVLLKAGADVNA

SEQ ID NO: 75 - protein, 33 aa, Artificial Sequence
  Ankyrin repeat module
KDQFGRTPLHIAAQSGHLEIVEVLLKAGADVNA

SEQ ID NO: 76 - protein, 33 aa, Artificial Sequence; identical to SEQ ID NO: 59
  Ankyrin repeat module

SEQ ID NO: 77 - protein, 33 aa, Artificial Sequence; identical to SEQ ID NO: 60
  Ankyrin repeat module

SEQ ID NO: 78 - protein, 33 aa, Artificial Sequence
  Ankyrin repeat module
KDFRGWTPLHLAAWSGHLEIVEILLKAGADVNA

SEQ ID NO: 79 - protein, 33 aa, Artificial Sequence
  Ankyrin repeat module
KDSVGYTPLHYAAHAGHLEIVEVLLKAGADVNA

SEQ ID NO: 80 - protein, 33 aa, Artificial Sequence; identical to SEQ ID NO: 40
  Ankyrin repeat module

SEQ ID NO: 81 - protein, 33 aa, Artificial Sequence
  Ankyrin repeat module
KDHVGYTPLHIAALAGHLEIVEVLLKAGEDVNA

SEQ ID NO: 82 - protein, 33 aa, Artificial Sequence
  Ankyrin repeat module
KDHKGRTPLHVAAAVGHLEIVEVLLKAGADVNA

SEQ ID NO: 83 - protein, 33 aa, Artificial Sequence
  Ankyrin repeat module
KDQQGVTPLHIAAITGHLEIVEVLLKAGADVNA

SEQ ID NO: 84 - protein, 33 aa, Artificial Sequence; identical to SEQ ID NO: 59
  Ankyrin repeat module

SEQ ID NO: 85 - protein, 33 aa, Artificial Sequence
  Ankyrin repeat module
KDVQGRTPLHIAAHSGHLEIVEVLLKVGADVNA

SEQ ID NO: 86 - protein, 33 aa, Artificial Sequence; identical to SEQ ID NO: 61
  Ankyrin repeat module

SEQ ID NO: 87 - protein, 33 aa, Artificial Sequence; identical to SEQ ID NO: 68
  Ankyrin repeat module

SEQ ID NO: 88 - protein, 33 aa, Artificial Sequence; identical to SEQ ID NO: 69
  Ankyrin repeat module

SEQ ID NO: 89 - protein, 33 aa, Artificial Sequence; identical to SEQ ID NO: 70
  Ankyrin repeat module

SEQ ID NO: 90 - protein, 33 aa, Artificial Sequence
  Ankyrin repeat module
KDEWGRTPLHLAAQYGHLEIVEVLLKAGADVNA

SEQ ID NO: 91 - protein, 33 aa, Artificial Sequence; identical to SEQ ID NO: 69
  Ankyrin repeat module

SEQ ID NO: 92 - protein, 33 aa, Artificial Sequence; identical to SEQ ID NO: 70
  Ankyrin repeat module

SEQ ID NO: 93 - protein, 33 aa, Artificial Sequence
  Ankyrin repeat module
KDTWGFTPLHIATESGHLEIVEVLLKAGADVNA

SEQ ID NO: 94 - protein, 33 aa, Artificial Sequence; identical to SEQ ID NO: 60
  Ankyrin repeat module

SEQ ID NO: 95 - protein, 33 aa, Artificial Sequence; identical to SEQ ID NO: 61
  Ankyrin repeat module

SEQ ID NO: 96 - protein, 277 aa, Homo sapiens
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 97 - protein, 173 aa, Artificial Sequence
  Extracellular domain of human CD40
EPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLR

SEQ ID NO: 98 - protein, 157 aa, Artificial Sequence
  Ankyrin repeat domain
DLGKKLLEAARAGQDDEVRILLAAGADVNAKDVLGWTPLHLAAFEGHLEIVEVLLKAGADVNAKDKKGWTPLQLAARTGHLEIVEVLLKAGADVNAKDHIGATPLHLAAWQGHPEIVEVLLKAGADVNAQDKSGKTPADLAADAGHEDIAEVLQKAA

SEQ ID NO: 99 - protein, 307 aa, Artificial Sequence
  Binding protein SMA014
GSDLGEKLLVAALYGQDDEVRELLKAGADVNAKDQWGLTPLHKAALQGHLEIVEVLLKAGADVNAKDERGHTPLHWAARFGHLEIVEVLLKAGADVNAQDQKGYTPADLAALWGHEDIAEVLQKAAGSPTPTPTTPTPTPTTPTPTPTGSDLGKKLLQAARAGQLDEVRELLKAGADVNAKDTWGFTPLHIAAESGHLEIVEVLLKAGADVNAKDVQGRTPLHIAAHSGHLEIVEVLLKAGADVNAKDFRGWTPLHLAAWSGHLEIVEILLKAGADVNAQDKSGKTPADLAARAGHQDIAEVLQKAA

SEQ ID NO: 100 - protein, 455 aa, Artificial Sequence
  Binding protein SMA087
GSDLGKKLLEAARAGQDDEVRELLKAGADVNAKDYFSHTPLHLAARNGHLKIVEVLLKAGADVNAKDFAGKTPLHLAAADGHLEIVEVLLKAGADVNAQDIFGKTPADIAADAGHEDIAEVLQKAAGSPTPTPTTPTPTPTTPTPTPTGSDLGEKLLVAALYGQDDEVRELLKAGADVNAKDQWGLTPLHKAALQGHLEIVEVLLKAGADVNAKDERGHTPLHWAARFGHLEIVEVLLKAGADVNAQDQKGYTPADLAALWGHEDIAEVLQKAAGSPTPTPTTPTPTPTTPTPTPTGSDLGKKLLQAARAGQLDEVRELLKAGADVNAKDTWGFTPLHIAAESGHLEIVEVLLKAGADVNAKDVQGRTPLHIAAHSGHLEIVEVLLKAGADVNAKDFRGWTPLHLAAWSGHLEIVEILLKAGADVNAQDKSGKTPADLAARAGHQDIAEVLQKAA

SEQ ID NO: 101 - protein, 603 aa, Artificial Sequence
  Binding protein SMA095
GSDLGKKLLEAARAGQDDEVRELLKAGADVNAKDYFSHTPLHLAARNGHLKIVEVLLKAGADVNAKDFAGKTPLHLAAADGHLEIVEVLLKAGADVNAQDIFGKTPADIAADAGHEDIAEVLQKAAGSPTPTPTTPTPTPTTPTPTPTGSDLGEKLLVAALYGQDDEVRELLKAGADVNAKDQWGLTPLHKAALQGHLEIVEVLLKAGADVNAKDERGHTPLHWAARFGHLEIVEVLLKAGADVNAQDQKGYTPADLAALWGHEDIAEVLQKAAGSPTPTPTTPTPTPTTPTPTPTGSDLGKKLLQAARAGQLDEVRELLKAGADVNAKDTWGFTPLHIAAESGHLEIVEVLLKAGADVNAKDVQGRTPLHIAAHSGHLEIVEVLLKAGADVNAKDFRGWTPLHLAAWSGHLEIVEILLKAGADVNAQDKSGKTPADLAARAGHQDIAEVLQKAAGSPTPTPTTPTPTPTTPTPTPTGSDLGKKLLEAARAGQDDEVRELLKAGADVNAKDYFSHTPLHLAARNGHLKIVEVLLKAGADVNAKDFAGKTPLHLAAADGHLEIVEVLLKAGADVNAQDIFGKTPADIAADAGHEDIAEVLQKAA

SEQ ID NO: 102 - protein, 488 aa, Artificial Sequence
  Binding protein SMA104
GSDLGEKLLVAALYGQDDEVRELLKAGADVNAKDQWGLTPLHKAALQGHLEIVEVLLKAGADVNAKDERGHTPLHWAARFGHLEIVEVLLKAGADVNAQDQKGYTPADLAALWGHEDIAEVLQKAAGSPTPTPTTPTPTPTTPTPTPTGSDLGKKLLQAARAGQLDEVRELLKAGADVNAKDTWGFTPLHIAAESGHLEIVEVLLKAGADVNAKDVQGRTPLHIAAHSGHLEIVEVLLKAGADVNAKDFRGWTPLHLAAWSGHLEIVEILLKAGADVNAQDKSGKTPADLAARAGHQDIAEVLQKAAGSPTPTPTTPTPTPTTPTPTPTGSDLGKKLLQAARAGQLDEVRELLKAGADVNAKDTWGFTPLHIAAESGHLEIVEVLLKAGADVNAKDVQGRTPLHIAAHSGHLEIVEVLLKAGADVNAKDFRGWTPLHLAAWSGHLEIVEILLKAGADVNAQDKSGKTPADLAARAGHQDIAEVLQKAA

SEQ ID NO: 103 - protein, 636 aa, Artificial Sequence
  Binding protein SMA091
GSDLGKKLLEAARAGQDDEVRELLKAGADVNAKDYFSHTPLHLAARNGHLKIVEVLLKAGADVNAKDFAGKTPLHLAAADGHLEIVEVLLKAGADVNAQDIFGKTPADIAADAGHEDIAEVLQKAAGSPTPTPTTPTPTPTTPTPTPTGSDLGEKLLVAALYGQDDEVRELLKAGADVNAKDQWGLTPLHKAALQGHLEIVEVLLKAGADVNAKDERGHTPLHWAARFGHLEIVEVLLKAGADVNAQDQKGYTPADLAALWGHEDIAEVLQKAAGSPTPTPTTPTPTPTTPTPTPTGSDLGKKLLQAARAGQLDEVRELLKAGADVNAKDTWGFTPLHIAAESGHLEIVEVLLKAGADVNAKDVQGRTPLHIAAHSGHLEIVEVLLKAGADVNAKDFRGWTPLHLAAWSGHLEIVEILLKAGADVNAQDKSGKTPADLAARAGHQDIAEVLQKAAGSPTPTPTTPTPTPTTPTPTPTGSDLGKKLLQAARAGQLDEVRELLKAGADVNAKDTWGFTPLHIAAESGHLEIVEVLLKAGADVNAKDVQGRTPLHIAAHSGHLEIVEVLLKAGADVNAKDFRGWTPLHLAAWSGHLEIVEILLKAGADVNAQDKSGKTPADLAARAGHQDIAEVLQKAA

SEQ ID NO: 104 - protein, 784 aa, Artificial Sequence
  Binding protein SMA099
GSDLGKKLLEAARAGQDDEVRELLKAGADVNAKDYFSHTPLHLAARNGHLKIVEVLLKAGADVNAKDFAGKTPLHLAAADGHLEIVEVLLKAGADVNAQDIFGKTPADIAADAGHEDIAEVLQKAAGSPTPTPTTPTPTPTTPTPTPTGSDLGEKLLVAALYGQDDEVRELLKAGADVNAKDQWGLTPLHKAALQGHLEIVEVLLKAGADVNAKDERGHTPLHWAARFGHLEIVEVLLKAGADVNAQDQKGYTPADLAALWGHEDIAEVLQKAAGSPTPTPTTPTPTPTTPTPTPTGSDLGKKLLQAARAGQLDEVRELLKAGADVNAKDTWGFTPLHIAAESGHLEIVEVLLKAGADVNAKDVQGRTPLHIAAHSGHLEIVEVLLKAGADVNAKDFRGWTPLHLAAWSGHLEIVEILLKAGADVNAQDKSGKTPADLAARAGHQDIAEVLQKAAGSPTPTPTTPTPTPTTPTPTPTGSDLGKKLLQAARAGQLDEVRELLKAGADVNAKDTWGFTPLHIAAESGHLEIVEVLLKAGADVNAKDVQGRTPLHIAAHSGHLEIVEVLLKAGADVNAKDFRGWTPLHLAAWSGHLEIVEILLKAGADVNAQDKSGKTPADLAARAGHQDIAEVLQKAAGSPTPTPTTPTPTPTTPTPTPTGSDLGKKLLEAARAGQDDEVRELLKAGADVNAKDYFSHTPLHLAARNGHLKIVEVLLKAGADVNAKDFAGKTPLHLAAADGHLEIVEVLLKAGADVNAQDIFGKTPADIAADAGHEDIAEVLQKAA

SEQ ID NO: 105 - protein, 784 aa, Artificial Sequence
  Binding protein
GSDLGKKLLEAARAGQDDEVRELLKAGADVNAKDYFSHTPLHLAARNGHLKIVEVLLKAGADVNAKDFAGKTPLHLAAADGHLEIVEVLLKAGADVNAQDIFGKTPADIAADAGHEDIAEVLQKAAGSPTPTPTTPTPTPTTPTPTPTGSDLGKKLLEAARAGQDDEVRELLKAGADVNAKDYFSHTPLHLAARNGHLKIVEVLLKAGADVNAKDFAGKTPLHLAAADGHLEIVEVLLKAGADVNAQDIFGKTPADIAADAGHEDIAEVLQKAAGSPTPTPTTPTPTPTTPTPTPTGSDLGEKLLVAALYGQDDEVRELLKAGADVNAKDQWGLTPLHKAALQGHLEIVEVLLKAGADVNAKDERGHTPLHWAARFGHLEIVEVLLKAGADVNAQDQKGYTPADLAALWGHEDIAEVLQKAAGSPTPTPTTPTPTPTTPTPTPTGSDLGKKLLQAARAGQLDEVRELLKAGADVNAKDTWGFTPLHIAAESGHLEIVEVLLKAGADVNAKDVQGRTPLHIAAHSGHLEIVEVLLKAGADVNAKDFRGWTPLHLAAWSGHLEIVEILLKAGADVNAQDKSGKTPADLAARAGHQDIAEVLQKAAGSPTPTPTTPTPTPTTPTPTPTGSDLGKKLLQAARAGQLDEVRELLKAGADVNAKDTWGFTPLHIAAESGHLEIVEVLLKAGADVNAKDVQGRTPLHIAAHSGHLEIVEVLLKAGADVNAKDFRGWTPLHLAAWSGHLEIVEILLKAGADVNAQDKSGKTPADLAARAGHQDIAEVLQKAA

SEQ ID NO: 106 - protein, 669 aa, Artificial Sequence
  Binding protein
GSDLGEKLLVAALYGQDDEVRELLKAGADVNAKDQWGLTPLHKAALQGHLEIVEVLLKAGADVNAKDERGHTPLHWAARFGHLEIVEVLLKAGADVNAQDQKGYTPADLAALWGHEDIAEVLQKAAGSPTPTPTTPTPTPTTPTPTPTGSDLGKKLLQAARAGQLDEVRELLKAGADVNAKDTWGFTPLHIAAESGHLEIVEVLLKAGADVNAKDVQGRTPLHIAAHSGHLEIVEVLLKAGADVNAKDFRGWTPLHLAAWSGHLEIVEILLKAGADVNAQDKSGKTPADLAARAGHQDIAEVLQKAAGSPTPTPTTPTPTPTTPTPTPTGSDLGKKLLQAARAGQLDEVRELLKAGADVNAKDTWGFTPLHIAAESGHLEIVEVLLKAGADVNAKDVQGRTPLHIAAHSGHLEIVEVLLKAGADVNAKDFRGWTPLHLAAWSGHLEIVEILLKAGADVNAQDKSGKTPADLAARAGHQDIAEVLQKAAGSPTPTPTTPTPTPTTPTPTPTGSDLGKKLLQAARAGQLDEVRELLKAGADVNAKDTWGFTPLHIAAESGHLEIVEVLLKAGADVNAKDVQGRTPLHIAAHSGHLEIVEVLLKAGADVNAKDFRGWTPLHLAAWSGHLEIVEILLKAGADVNAQDKSGKTPADLAARAGHQDIAEVLQKAA